(19) United States
(12) Patent Application Publication
(10) Pub. No.: US 2010/0189701 A1
(43) Pub. Date: Jul. 29, 2010

(54) METHODS AND COMPOSITIONS TO TREAT ARRHYTHMIAS

(76) Inventors: Ira S Cohen, Stony Brook, NY (US); Peter R. Brink, Setauket, NY (US); Michael R. Rosen, New York, NY (US); Richard B. Robinson, Cresskill, NJ (US); Peter Danilo, JR., Hopewell, NJ (US); Heather S. Duffy, Bronx, NY (US)

Correspondence Address: KENYON & KENYON LLP, ONE BROADWAY, NEW YORK, NY, 10004, US

(21) Appl. No.: 12/520,804

(22) PCT Filed: Dec. 26, 2007
(86) PCT No.: PCT/US07/26475

§ 371 Date: Mar. 3, 2010

(51) Int. Cl. A61K 35/12; A61K 31/7088; A61K 31/5377

(52) U.S. Cl. 424/93.21; 514/44 R; 514/234.5; 514/44 A

(57) ABSTRACT

The present invention provides compositions and methods of treatment for atrial fibrillation and ventricular tachycardia. The compositions are useful for modifying the conducting properties of heart tissues in which impulses are generating and/or are useful for altering refractoriness without prolonging repolarization.

### BACKGROUND OF THE INVENTION

Cardiac arrhythmia is a group of conditions in which the muscle contraction of the heart is irregular and/or is faster or slower than normal.

Arrhythmias stem from several causes. The heart's natural timekeeper—a small mass of specialized cells called the sinus node—iniates and maintains the heart's normal rhythm, which is referred to as normal sinus rhythm. However, the sinus node can malfunction and develop an abnormal rate or rhythm of electrical impulse initiation. However, because all heart tissue not only propagates the normal rhythm of the heart, but is capable of initiating a beat, any part of the heart muscle can interrupt the normal sinus rhythm, or even take over as the heart's pacemaker, setting off an abnormal heartbeat. When one of these events interrupts the heart's normal beat, either intermittent or sustained arrhythmias can occur.

Introduced in England in 1785, digitalis (its modern-day derivative is digoxin) still remains a treatment for fast heart rates caused by atrial fibrillation. While it does not slow the fibrillation in the atrium, it does decrease the number of rapid, irregular beats that reach the ventricles. Hence, it improves the function of the ventricles to pump blood to the body in this setting. Several new compounds developed since the 1950s are used to stabilize the heartbeat or as preventive therapy to avert complications: warfarin, an anticoagulant, is used in atrial fibrillation patients to prevent stroke-inducing blood clots; antiarrhythmic agents such as amiodarone and sotalol help maintain the heart's normal rhythm; beta blockers such as metoprolol and atenolol limit the stimulating effects of adrenaline and, secondarily calcium, on the heart, and slow the heart rate in atrial fibrillation; and calcium channel blockers such as verapamil and diltiazem help slow the heart rate and suppress tachycardias. Patients with more serious, potentially lethal rapid heartbeat abnormalities have a different option that has dramatically improved their chances of survival—an implantable cardioverter/defibrillator (ICD). An ICD is inserted surgically, just as a pacemaker is. The ICD constantly monitors heart rhythm, and when it senses that the heart is undergoing a potentially lethal arrhythmia, the ICD gives the heart a shock to return the rhythm to normal.

A third tool is catheter ablation. This method returns rapid, irregular heartbeats to normal by using a catheter to deliver radiofrequency energy that destroys a region or regions of heart-muscle cells. The resulting scar cuts off the route of the arrhythmic beats. This technique has enabled many patients to live a life free of both medicines and recurrent bouts of arrhythmia, such as those caused by reentrant atrioventricular nodal tachycardia or Wolff-Parkinson-White (WPW) syndrome.

Although device therapy and ablation have made major advances over the past twenty years, in many ways outstripping cardiac antiarrhythmic drugs, two arrhythmias remain highly problematic: (1) ventricular tachycardia/fibrillation and 2) atrial fibrillation. Ventricular tachycardia leading to sudden cardiac death (SCD) occurs in about 200,000-400,000 individuals/year in the United States and accounts for up to 20% of all deaths in adults. The treatment for ventricular tachycardia is often the administration of an ICD. While use of ICDs in primary SCD prevention is life-saving, it is also inefficient and costly; and drug therapy alone remains unsatisfactory. Atrial fibrillation (AF) currently afflicts about 2.3 million Americans and may reach 15 million by 2050. Patients with AF are at increased risk for stroke, heart failure, and death. Even the latest surgical and catheter ablation techniques used to treat AF are not yet satisfactory. As for drug therapy, Na channel blocking agents (e.g. flecainide) and ERG blockers (e.g. dofetilide) are effective in specific settings but long-term results are disappointing as AF is relentlessly progressive. Moreover, the channel-blocking drugs have a very real incidence of proarrhythmic complications. Recent results with “upstream therapies” such as ACE inhibitors and AT-1 receptor blockers show promise in reducing recurrences of paroxysmal AF, but these approaches fall short of providing the broad protection against AF that is desirable.

Antiarrhythmic gene and cell therapies are a nascent field. As such there is need for considerable information about applicability to specific arrhythmias, extent and duration of efficacy, safety, means for delivery, and comparison with standard device and drug therapies. The leading edge of research here to date has related to treatment of bradyarrhythmias using biological pacemaking and to a lesser extent AV bridging. In brief, strategies have included overexpression of the beta-2 adrenergic receptor, transfection with a dominant negative construct to reduce I_{K1}, overexpression of the HCN gene family to increase pacemaker current and use of mutagenesis to create designer pacemaker genes. Investigators have used fusion of myocytes with other cell types via application of polyethylene glycol, insertion of the channel proteins that can generate an action potential in normally non-excitable cells, and engineering of K channels to provide them with a subset of the properties of HCN channels. In the area of cell therapy, it has been shown that human embryonic stem cells can be coaxed into a pacemaker line, and that adult human mesenchymal stem cells can be used as platforms to carry pacemaker genes. Finally, attempts have been made to bridge the atria and ventricles such that in the setting of AV block and normal sinus node function impulses initiated in the atrium can be carried to the ventricles either via artificially fabricated bypass tracts or via upregulated Ca channels to improve conduction through the node.

Hence, in the treatment of unacceptable bradycardia or AV block, gene and cell therapies can be, and have been, brought to bear and a subset of these techniques is moving ahead rapidly towards possible clinical application either as monotherapy or more likely in a tandem approach with electronic pacemaker therapy.

In contrast to the diversity of strategies for bradyarrhythmias, the treatment of tachyarrhythmias has seen a far more focused and even limited approach due to the unique challenges posed by tachyarrhythmias. Much of the research to date on gene therapy of tachyarrhythmias has been done by the Marban and Donahue groups at Johns Hopkins and Case Western, respectively, and additional work has been reported by the Gepstein group at the Technion. The Donahue/Marban approach has explored a variety of means for optimizing gene delivery to tissues, both regionally and globally. They have also reported the use of pluronic and trypsin to bathe the atrial epicardium with a slurry that facilitates access of viral vectors carrying genes of interest to fibrillating atria: the intent would be to hyperpolarize the atria using genes of the Kir2.1 and 2.2 family to increase I_{K1}. Nevertheless, although this approach facilitates gene delivery, it promotes excessive inflammation. The Hammond group as well as the Donahue/Marban groups have also experimented with various permeabilizing agents (serotonin, histamine, etc.) as well as with VEGF to facilitate gene delivery. Cooling of the heart and aortic cross-clamping have been used as additional aids to localizing gene delivery, but these are viewed as excessive for eventual clinical application. About the best success to date has seen about 50% of cells in any region transfected, with viral transfer being diffusion-limited and especially problematic in the ventricles.

Perhaps the most productive area of investigation has been in AF. Here, attempts have been made to overexpress G proteins in the hope of amplifying vagal tone on the AV node and slowing AV conduction or implanting the nodal region with fibroblasts to induce scarring and AV block. While these approaches are appropriate for producing rate control (rather than rhythm control) whether they will offer a useful alternative to Rf ablation is uncertain. It is for this reason that the methods of the present invention relating to treating AF focuses more on the reentrant mechanism and the maintenance of sinus rhythm than on blocking conduction to the ventricle.

Of particular relevance to our ventricular tachycardia research is the recent report by the Donahue group of delivery via vascular infusion to a peri-infarct zone of pigs of a dominant negative HERG mutant (HERG-G628S) or connexin43. Whereas a monomorphic ventricular tachycardia was consistently inducible in the infarcted animals before gene transfer, one week later 4 of 5 Cx43-transferred animals and 5 of 5 HERG-G628S-transferred animals showed no such arrhythmia. Interestingly, conduction velocity was reported as improved in the Cx43-recipient animals while ventricular septal MAP and ERP were increased in the HERG-G628S recipient animals. Although this is a preliminary report, the results are exciting as they indicate the feasibility of a local approach to VT therapy in the chronic infarct setting.

Tempering excitement regarding the gene therapy approach using viral vectors are concerns with regard to inflammation. Additional concerns about viruses relate to episomal or limited genomic expression of genes, although these concerns are of lesser magnitude in proof-of-concept experiments. Because of these concerns, the present inventors believe the use of hMSCs as platforms for gene delivery is a complementary intervention worthy of exploration. The observation by the present inventors and others that hMSCs cells can be loaded with specific gene constructs and can be used to deliver them without the concerns raised by viral therapy is quite exciting. But cell therapies, too, have shortcomings in terms of long term application (as regards their migration to other sites, differentiation into other cell types, and long-term expression of genes of interest). However, the hMSCs use in the present invention satisfy some concerns in that they are not immunogenic, and—as late-passage cells—appear not to differentiate into other cell types. Moreover, use of quantum dots also assist in studying the localization of cells to the site of administration.

To summarize, no viral vector-based therapy has yet been demonstrated to be clinically applicable and most experiments reported have been intended not so much for clinical application but as proof of concept that gene therapies can be of use. With regard to cell therapies, these have been much more of the type intended to regenerate and repair myocardium than to be specifically antiarrhythmic. The repair and regeneration field has also seen clinical application with autologous and allogeneic adult mesenchymal stem cells. However, the success of these approaches has been modest at best, with safety having been much more clearly demonstrated than any clear therapeutic benefit. Moreover several of the leading stem cell advocates for repair and regenerative therapies have called for more preclinical research before further attempts are made to apply such therapy to man (e.g. Douglas Losordo, Kenneth Chien).

Accordingly there remains a need for effective therapies and compositions to treat ventricular tachycardia and atrial fibrillation. The present invention fulfills this need.

### SUMMARY OF THE INVENTION

The present invention provides a method of treating atrial fibrillation comprising modifying the conducting properties of the tissues in which the impulses are propagating, the method comprising improving conduction in the tissues by increasing gap junctional conductance in said tissues.

Increasing gap junctional conductance may mean, but is not limited to increasing numbers of gap junctions (i.e. overexpressing), may mean providing exogenous connexins that are “better” in forming gap junctions, or may mean providing a gap junction that couples to another cell at a faster rate.

Increasing gap junctional conductance may thus comprise delivering to said tissues hMSCs transfected with endogenous heart connexins, such as Cx40, Cx43 or Cx45. In certain embodiments, increasing gap junctional conductance comprises delivering said tissues a viral vector, such as a vector derived from a lenti virus, capable of expressing endogenous heart connexins in said tissue.

In another embodiment, increasing gap junctional conductance comprises administering a chemical stimulator of connexin expression to said tissues to cause said tissues to overexpress endogenous connexins selected from the group consisting of Cx 40, 43, 45. Exemplary chemical stimulator include 4PB or Zp123. In certain embodiments, increasing gap junctional conductance comprises administering a MMP-7 inhibitor, such as Gefitinib, to cause said tissues to overexpress endogenous connexins selected from the group consisting of Cx 40, 43, and 45.

In other embodiments, increasing gap junctional conductance comprises delivering to said tissues hMSCs transfected with exogenous connexins selected from the group consisting of Cx46 and Cx32. In other embodiments, increasing gap junctional conductance comprises administering to said tissues a viral vector capable of expressing an exogenous heart connexin in said tissue, wherein the exogenous connexin is selected from the group consisting of Cx46 and Cx32. Preferred viral vectors include those derived from a lentivirus.

Methods of the present invention also provide a method of treating atrial fibrillation comprising prolonging the refractory period by slowing deactivation of the delayed rectifier in the atrium. In certain embodiments, the method comprises delivering the ERG1 gene or a mutant ERG1 gene having slower deactivation kinetics as compared to ERG1 to the atrium without co-expression of MiRP1. Preferred mutant ERG1 genes include K538A and L539W. In certain embodiments, the ERG1 or mutant ERG1 is delivered via delivering hMSCs transfected with ERG1 or mutant ERG1 or via delivering a viral vector (such as a lenti-viral vector) capable of expressing ERG1 or the mutant ERG1.

In other embodiments, prolonging the refractory period by slowing deactivation of the delayed rectifier in the atrium comprises delivering siRNA to the atrium to silence native MiRP1 expression. The siRNA may be delivered via hMSCs transfected with a connexin capable of allowing delivery of siRNA through its gap junctions to cells of the atrium.

In other embodiments, prolonging the refractory period by slowing deactivation of the delayed rectifier in the atrium comprises delivering of a mutant form of ERG1, said mutant form having slowed deactivation kinetics as compared to a wild type ERG1. Exemplary mutants include K538A and L539W.

Another embodiment of the present invention provides a method of treating atrial fibrillation comprising locally reducing gap junction conductance by overexpressing Cx31.9 to prevent rapid impulse initiation that is focally triggered from propagating beyond its site of origin. Cx31.9 may be delivered via a viral vector capable of expressing Cx31.9. The viral vector or hMSCs may be injected intramyocardially in the base of the left atrial appendage.

The present invention also provides compositions useful for treating atrial fibrillation. Exemplary compositions comprising a hMSC transfected with, or a viral vector capable of expressing Cx40, Cx43, Cx45, Cx 46, Cx32, ERG1, a mutant ERG1 (as discussed above), Cx31.9, or MiRP1 siRNA. The present invention also provides a composition for treating atrial fibrillation comprising MiRP 1 siRNA.

The present invention further provides a method of treating ventricular tachycardia by increasing conduction velocity in areas of slow conduction by altering Na channel availability to provide a greater number of Na channels to be activated from the depolarized membrane potential. In certain embodiments, increasing conduction comprises providing a hMSC or a viral vector expressing a sodium channel having a more positive midpoint for steady state inactivation as compared to a normal heart Na channel. An exemplary sodium channel is a skeletal muscle sodium channel SCN4a or a mutant sodium channel, SCN4a-1306E.

Another embodiment of the present invention provides a method of treating ventricular tachycardia by suppressing conduction completely in a desired location of the ventricle, comprising administering to the desired area siRNA against Nav1.5 alpha subunit to induce bidirectional conduction block at the area.

The present invention further provides a method of treating ventricular tachycardia by enhancing conduction by increasing membrane potential of ventricular myocytes. In certain embodiments, enhancing conduction comprises delivering to the ventricular myocytes the ERG3 gene, either by delivering hMSCs or viral vectors expressing the ERG3 gene. In other embodiments, enhancing conduction by increasing membrane potential comprises delivering to the ventricular myocytes an inward rectifier gene, such as Kir 2.1 or 2.2 or a combination thereof. Preferred delivery is via transfected hMSCs or viral vectors expressing an inward rectifier gene.

Another embodiment of the present invention provides a method of treating ventricular tachycardia by prolonging refractoriness by slowing deactivation of the delayed rectifier in the ventricle. In certain embodiments, prolonging refractoriness by slowing deactivation of the delayed rectifier in the ventricle is achieved by delivery the ERG1 gene to the ventricle without co-expression of MiRP1. Preferred delivery is achieved via delivering hMSCs transfected with ERG1 or via delivering a viral vector capable of expressing ERG1. Preferred viral vectors are derived from a lenti virus. In other embodiments, prolonging the refractory period by slowing deactivation of the delayed rectifier in the ventricle comprises delivering siRNA to the ventricle to silence native MiRP1 expression. The siRNA may be delivered via hMSCs transfected with a connexin capable of allowing delivery of siRNA through its gap junctions to cells of the ventricle.

In another embodiment, prolonging the refractory period by slowing deactivation of the delayed rectifier in the ventricle comprises delivering of a mutant form of ERG1 (via transfected hSMCs or viral vector), said mutant form having slowed deactivation kinetics as compared to a wild type ERG1. Exemplary ERG1 mutants include K538A and L539W.

The present invention also provides compositions for treating ventricular tachycardia comprising a hMSC transfected with SCN4a, SCN4a-1306E, ERG3, Kir2.1, Kir 2.2, ERG1, a mutant ERG1. Further provides are compositions for treating ventricular tachycardia comprising a viral vector capable of expressing in heart tissue SCN4a, SCN4a-1306E, ERG3, Kir2.1, Kir 2.2, ERG1, a mutant ERG1. Other compositions include a composition for treating ventricular tachycardia comprising MiRP1 siRNA or Nav1.5 alpha siRNA.

### BRIEF DESCRIPTION OF THE FIGURES

FIG. 1: A: Upper panel, leads I-III and a high right atrial electrogram during normal sinus rhythm from a dog post-surgical scar induction. Lower panel, termination of burst atrial pacing (at arrow) followed by persistence of atrial flutter-fibrillation, characterized by regularly repetitive P waves interspersed with additional ectopic atrial activity. B: Upper panel, sinus rhythm followed by onset of left atrial pacing. In lower panel, termination of pacing is followed by an irregularly irregular atrial tachyarrhythmia associated with an irregular ventricular response, consistent with atrial fibrillation.

FIG. 2: Example of non-contact mapping of left ventricle with CARTO system in a dog with complete heart block, an electronic right ventricular apical endocardial pacemaker, and an HCN2 adenoviral construct administered into the proximal left bundle branch system. The lower panels demonstrate four projections, showing early activation (red) of the LV septum via the electronic pacemaker. Late activation is blue. The upper panels show an impulse activating LV endocardium at several sites simultaneously, reflecting the arrival of an impulse initiated in the left bundle branch system.

FIG. 3: Method and representative map of biventricular activation using our epicardial mapping system. Left: Diagram showing the positioning of the epicardial electrode sheets (hatched areas) and of the transmural needles (only depicted on the cut edge of the heart, top). The heart is presented as if incised through the posterior side of the septum and folded open. In each individual heart electrode positions are classified into 18 regions. RV, right ventricle; LV, left ventricle; basal; mid; apical; RA, right anterior, RL, right lateral; RP, right posterior; LA; left anterior; LL, left lateral; LP, left posterior. Pulse symbol indicates the position of a stimulating electrode. Needle electrode positions (n) are indicated by black dots. Two transmural needles are in reality inserted at each position. Each epicardial electrode sheet harbors 14 epicardial electrodes. Right: Isopotential maps from the epicardial surface of right (RV) and left ventricle (LV) at the moment of the peak of the T-wave in the body surface ECG of a dog. Electrograms are recorded from the sites indicated (A-B).

FIG. 4: Example of loading hMSC with a gene of interest, studying it biophysically and determining its effect on the heart. Panels A-C study functional expression of I_{f} in hMSCs transfected via electroporation with mHCN2 gene. I_{f} was expressed in hMSCs transfected with the mHCN2 gene (B) but not in nontransfected stem cells (A). C, Fit by the Boltzmann equation to the normalized tail currents of I_{f} gives a midpoint of −91.8+/−0.9 mV and a slope of 8.8+/−0.5 mV (n=9). I_{f} was fully activated around −140 mV with an activation threshold of −60 mV. Inset shows representative tail currents used to construct I_{f} activation curves. Voltage protocol was to hold at −30 mV and hyperpolarize for 1.5 seconds to voltages between −40 and −160 mV in 10-mV increments followed by a 1.5-second voltage step to +20 mV to record the tail currents. Panels D-E show function of hMSCs loaded with HCN2 gene 10 days after injection into canine LV anterior wall in dog in complete heart block. Panels D, placebo experiment, dog injected with saline. Left panel shows pacing from injection site at the time of surgery, right panel shows idioventricular rhythm having a slow rate (about 42 bpm) and a completely different QRS vector from the paced beats at the injection site. Panels E, dog injected with approximately 1.2 million hMSCs loaded with HCN2. Left panel shows paced beats at injection site at time of hMSC administration. Right panel 10 days later shows rhythm having same QRS vector and rate around 60 bpm.

FIG. 5: Use of the surfactant, Pluronic 127 (poloxamer, 5 ml of a 20% solution) containing 0.5% trypsin as a vehicle for loading adenovirus into myocardial cells. A solution containing adenovirus as a vector for green fluorescent protein was painted directly onto the atrial myocardium. Panels A and B (×100). A, Control H and E stain showing normal epi- and myocardium. Panel B, H and E stain showing inflammatory cell infiltrate following administration of pluronic and trypsin with viral construct. Panel C: Control peroxidase staining for GFP in normal tissue, showing no peroxidase reaction. Panel D, positive staining for peroxidase at site of administration of pluronic plus viral construct. Panels A,B, ×100; C and D, ×400.

FIG. 6: H and E staining of sites of injection of electroporated hMSCs at 2 and 6 weeks after injection. Top panels: left, at 2 weeks abundant undifferentiated hMSCs are noted in-between myocardial bundles. Many hMSCs show nuclear budding and hyperchromasia (arrow) typical for cell death by apoptosis. Edema is present as well. Right, At six weeks only smaller numbers of undifferentiated hMSCs remain (B), but none display apoptotic features. No edema is seen. Middle panels: Left: hMSCs are easily noted (arrow) adjacent to the cicatricial fibrosis of this six-week-old needle tract. Right: The hMSCs identified morphologically consistently show cytoplasmic immunolabelling (brown reaction product, arrow) for GFP. Bottom panels: Left: hMSCs (arrow) do not display labeling/binding of dog-immunoglobulin to their surface, which is evidence against humoral rejection. Right: CD3 positive T lymphocytes (arrow) are rarely noted in association with clusters of hMSCs; their paucity is evidence against cellular rejection (Original magnification: ×400).

FIG. 7: Quantum dot (QD) tracking of hMSCs. In this experiment, the use of quantum dots loaded passively into 120,000 hMSCs to identify the location of the hMSCs in the rat heart is shown. One hour later fixed, frozen sections were cut transversely (b, inset) at 10 μm and mounted onto glass slides. Sections were imaged for QD fluorescence emission at 655 nm with phase overlay to visualize tissue borders. Upper, QD-hMSCs can be visualized at a low power. Note their relationship to the needle track and to the small amount of crazy glue applied over the site of injection. Middle panel: Serial low power images are registered with respect to one another and binary masks are generated, where white pixels depict all of the QD+ positive zones in the images. The binary masks for each QD+ section of the heart are compiled and used to generate the 3D reconstruction of delivered cells in the tissue. Bottom panel shows the reconstruction of the spatial localization of QD-hMSCs in the heart, which permits further quantitative analysis. One parameter that can be computed is the distance of individual cells from the centroid of the total cell mass.

FIGS. 8A and 8B show structure function studies using alanine or trytophan scanning mutagenesis. A number of mutant hERG channels had substantially slower deactivation kinetics than wild type hERG channels.

FIG. 9 shows expression of mHCN2 in human mesenchymal stem cells.

FIG. 10 shows that the beta agonist isoproterenol has a direct effect on the expressed current.

FIG. 11 demonstrates that acetylcholine only has an effect in the presence of the beta agonist (accentuated antagonism).

FIG. 12 shows the time course of coupling in vitro between stem cells and either neonatal rat (filled circles) or adult canine (open circles) ventricular myocytes.

FIG. 13 illustrates the protocol used to assess the effectiveness of coupling as a function of the gap junctional conductance.

FIG. 14 relates the magnitude of gap junctional conductance to the effectiveness of transfer of HCN2 induced current.

FIG. 15 shows the expression of this current in hMSCs where its midpoint of inactivation is −62 mV.

FIG. 16 shows that ERG3 has the largest steady state conductance as compared to other ERG family members.

FIG. 17A shows the expression of the inward rectifiers Kir2.1 in human mesenchymal stem cells and FIG. 17B shows the expression of the inward rectifiers Kir2.2 in human mesenchymal stem cells.

FIG. 18 shows the transfer over time of a morpholino of length 12 bases between two cells expressing Cx43.

FIG. 19 shows sample data from two cells, one in which HCN2 was expressed and 96 hours later the cell was patch clamped, and a second cell which was first transfected with HCN2 and 48 hours later siRNA was delivered by lipofectin.

FIG. 20 shows the results of a study where neonatal and adult rat ventricular myocytes were infected with an adenovirus carrying the pacemaker gene HCN2. The voltage dependence of activation was dramatically affected by the cell background being about 20 mV more positive in the neonatal myocytes.

FIGS. 21A and 21B shows that early and late passage hMSCs can be effectively transfected with a transgene by electroporation.

FIG. 22 shows that both early and late passage hMSCs can express abundant levels of Cx43.

FIGS. 23A and 23B show that although early passage hMSCs can be effectively induced to differentiate along an adipogenic lineage such is not the case for late passage hMSCs.

FIG. 24 shows that DNA laddering characteristic of apoptotic cells is absent from both early and late passage hMSCs.

FIG. 25 shows that caspase activation is also not higher in later passages hMSCs.

FIG. 26A shows that late passage cells proliferate less readily than those from earlier passages although cell division is still measurable. FIG. 26B shows that BrdU incorporation is markedly reduced in later passage hMSCs.

FIG. 27 illustrates that the previously used techniques of electroporation or lipid mediated transfection result in non-uniform loading of QDs while hMSCs loaded with quantum dots by a novel passive loading technique are uniformly labeled.

FIG. 28 shows low magnification images for the 1 hour animal at the plane of the stem cell injection illustrating the ease with which the fluorescence of the red quantum dots can be observed in the needle track above background autofloresence.

FIG. 29A provides an image from a 1 day animal. Again even at low power the QD flouresence is easily observed above autofloresence. Over 100 sections were studied and custom algorithms were written to reconstruct the locations of all QD labeled cells. FIG. 29B shows binary maps at three levels used for 3-D reconstruction. FIG. 29C provides this three dimensional picture. Finally also illustrated in FIG. 29D is the distance of each of the stem cells from the centroid of the stem cell mass.

FIG. 30 illustrates schematically how a re-entrant arrhythmia might arise and how alteration of longitudinal resistance could abolish such an arrhythmia.

FIG. 31 illustrates the concept of cellular delivery of siRNA. The cell on the left in FIG. 31 has been transfected with a cDNA for a hairpin siRNA (shRNA). The action of Dicer RNAse (common to all cells) produces an dimerized siRNA which is in equilibrium with single stranded forms. The right-hand cell represents a wild type cell receiving siRNA via gap junction.

FIG. 32 shows a schematic for dual whole cell patch clamp experiments, along with the imaging approach to allow for simultaneous measurement of gap junctional membrane conductance and the permeation of fluorescently tagged probes. Samples of types of data that can be collected are illustrated in FIGS. 32A-E.

FIG. 33 shows that junctional conductance between cell pairs can be measured. FIG. 33a shows the time course of pH uncoupling for a canine ventricular myocyte pair. FIG. 33c is a fluorescent image of a canine ventricular myocyte cell pair imaged during the bubbling of 100% CO2. FIG. 33b shows a fluorescent image while 33c shows the data from the experiment in 4b where junctional conductance and fluorescent intensity are plotted vs. time during exposure to 100% CO2.

FIG. 34 illustrates a Western blot where HeLa cells expressing Cx43 have been exposed to increasing doses of 4PB. These data demonstrate that up-regulation of connexins is possible via pharmacological intervention.

FIG. 35 is a figure published in Valiunas et al., (2004) illustrating stem cell to canine myocyte coupling mediated by Cx43.

FIG. 36 shows the time course under control conditions for a variety of cell types with isolated canine or rodent ventricular myocytes. The time constant for half maximal junctional conductance under in vitro conditions is 24 hours.

FIG. 37 is a time-lapse series of the transfer of a oligonucleotide 12 bases in length (12 mer) from one cell of a pair to another where the junctional conductance was 40 nS during an experiment.

FIG. 38 is a summary graph of oligonucleotide permeability for nucleotides of different lengths, TEA and Lucifer Yellow relative to K ion for Cx43. Cell types expressing Cx43 include hMSCs. The data are taken from Valiunas et al., 2002; 2005; Goldberg et al., 2004; Weingart, 1974).

FIG. 39 shows transfer of 12 mer between a HeLa cell pair expressing Cx40 (Valiunas et al., 2002).

FIG. 40 shows the summary histogram taken from our recent publication (Valiunas et al., 2005).

FIG. 41: Effect of HCN2 over-expression in neonatal rat ventricular myocyte culture. A)

Representative spontaneous action potentials from a culture exposed to a GFP expressing adenovirus. B) Representative action potentials from a culture exposed to an HCN2 expressing adenovirus. C) Summary data on spontaneous rate, slope of phase 4 depolarization and maximal diastolic potential (MDP) control cultures (Ctrl), HCN2 expressing cultures and GFP expressing cultures, demonstrating a significant effect of HCN2 expression on all 3 parameters. * indicates P<0.05.

FIG. 42: Cell coupling between cell pairs. Top) Fractional current transfer as a function of measured junctional conductance in Cx43 expressing N2A cell pairs, indicating that optimal current transfer occurs when junctional conductance is in the 10-15 nS range. Bottom) Time course of development of junctional coupling in myocyte-mesenchymal cell pairs, indicating at junctional conductance of 10-15 nS occurs 2-3 days after establishment of the co-culture.

FIG. 43: Comparison of in vitro and in vivo effects of expressed HCN channel mutations. Left) Adenoviruses expressing HCN2 and the E324A mutation of HCN2 have equivalent effects on rate in culture (top) and in the intact canine heart (bottom). An adenovirus expressing a chimeric channel consisting of the transmembrane domain of HCN1 and the N- and C-termini of HCN2 causes bursts of rapid beating and pauses in culture (top) and in the intact canine heart (bottom).

FIG. 44: The action potential of neonatal rat ventricular cells maintained in culture is Na current dependent and constant over time. Top) The action potential upstroke is rapid (>100 V/s) and TTX sensitive, indicating a contribution of Na current to the upstroke. Bottom) Action potential maximum rate of rise (upstroke) and maximum diastolic potential (MDP) are constant in culture during a 2-8 day period.

FIG. 45: I-V and Inactivation relations of native cardiac Na current in neonatal rat myocytes. Left) The I-V relation of the native Na current from neonatal myocytes. Right) The inactivation relation under of the native Na current from neonatal myocytes, confirming significant Na channel availability at typical resting potentials for these cells.

FIG. 46: Effect of Na channel block and membrane depolarization on conduction velocity measured in neonatal rat ventricular myocyte cultures. A) Color coded maps of conduction velocity in a culture grown on a multi-electrode array and stimulated from below under control conditions (left) and in the presence of 1 μM tetrodotoxin (TTX, right). Conduction velocity decreases 20% in this culture with this TTX concentration. B) Conduction velocity in a culture stimulated from below under control conditions (5.4 mM K, left) and in the presence of elevated K (10 mM, right). Conduction velocity decreases 27% in this culture in elevated K. C) Summary data of the effect of a range of TTX concentrations (left) and K concentrations (right) on conduction velocity.

FIG. 47: Establishment of cell coupling and synchronization across a gap in a neonatal rat ventricular culture. Cells were plated onto a multi-electrode array with a vertical barrier (purple line in left panel) initially separating the cells into two regions. Left) At day 1 after removal of the barrier the two regions of myocytes are not physically connected and beat spontaneous at different rates, showing no synchronization. Right) Three days after removing the barrier the two regions now beat synchronously, demonstrating effective coupling across the region where the barrier was originally located.

FIG. 48: Expression of the SKM1 skeletal isoform of the Na channel in neonatal rat cardiac myocytes. Cells were transfected by electroporation either with GFP alone or with GFP and SKM1, and recordings were then made several days later from GFP expressing cells. Top) A family of current traces under control conditions (left) from a cell only expressing GFP. The SKM1 selective toxin μ-CTX GIIIA was used (200 nM) to block any SKM1 current, and there was no effect (red trace) when tested at a single voltage. Note the faster inactivation in the family of traces (left) in this cell compared to the GFP only cell. Middle) Similar experiment as in the top panel, but in a cell expressing both GFP and SKM1, illustrating the effectiveness of the toxin in reducing the current. Bottom) The family of current traces (left) of the residual Na current after toxin exposure in the cell from the middle panel, illustrating the slower inactivation typical of the cardiac isoform. The steady-state inactivation relation from the SKM1 expressing cell (right) is shifted to more negative potentials after the toxin blocks the SKM1 contribution, leaving the native cardiac Na current.

FIG. 49: Effect of K depolarization on action potential upstroke in neonatal myocytes in the absence and presence of SKM1 expression. Top) The left panel shows an action potential from a cell exposed to a GFP expressing adenovirus in 5.4 mM external K (black) and 10 mM K (red). The right panel shows the action potential upstroke and its first derivative on an expanded time scale. The elevated K reduced the upstroke to 68% of its control value (mean value after elevated K is 56% of control). Bottom) In an SKM1 expressing myocyte the upstroke is better preserved, measuring 83% of its control value in elevated K.

FIG. 50 shows patch clamp studies on hMSCs expressing SkM1. Left: An hMSC transfected with SkM1 was held at potentials between −100 mV and −70 mV and pulsed to 0 mV. Since the current induced from −70 mV is > half the magnitude of the current induced from −100 mV the midpoint of inactivation was positive to −70 mV. Right: hMSCs were held at holding potentials between −100 and −55 mV for 1.5 sec and pulsed to 0 mV for 150 msec. The Boltzmann 2-state fit gives a midpoint of −62.4±3.2 mV and a slope factor of −10.3±3.2 mV.

FIG. 51 shows the effect of low and high TTX concentrations on CV in GFP and SkM1 expressing cultures. Control CV did not differ between GFP and SkM1 expressing cultures. However, there is a significantly greater effect of 100 nM TTX, which selectively inhibits skeletal Na channels relative to cardiac ones, to decrease CV in SkM1 expressing cultures compared to GFP expressing cultures. Following 30 μM TTX, which blocks both isoforms, CV is equivalently reduced; n=6 for GFP; n=7 for SkM1. * P<0.05 relative to matched control; ** P<0.05 relative to other group under same conditions.

FIG. 52 shows the effect of K depolarization on CV and APupstroke in neonatal myocytes in the absence and presence of SkM1 expression. Top) In GFP but not SkM1 cultures there is a significant effect on CV of increasing K from 5.4 to 10 mM. When 1 μM μ-CTX is added in 10 K, CV is reduced in SkM1 cultures to the level recorded in GFP cultures. Bottom) Increasing K to 10 mM causes significant decrease in Vmax in GFP but not SkM1 expressing cells. In SkM1 expressing cells, Vmax remains >100 V/s in 10 K. * P<0.01; n=6 in each group.

FIG. 53 shows an optical recording of CV (left) and maximum pacing frequency (right). Adenoviral treated neonatal myocyte cultures expressed SkM1 or GFP and were studied in normal and high (10.4 mM) K. Left: The effectiveness of SkM1 expression to maintain high CV persists as pacing frequency increases. Right: SkM1 treatment resulted in higher resistance to rhythm instabilities, i.e. higher break-point frequencies were noticed compared to GFP cultures in high K.

FIG. 54 shows the dependence of Vmax on MDP in LV epicardium of infarcted mice injected with saline-adenovirus (n=3) or SkM1 (n=3) adenovirus. BCL=250 ms. *P<0.05.

FIG. 55 shows the induction and persistence of VT in dog 1223 (infarcted and injected with GFP adenovirus). Panel A: ventricular pacing with insertion of premature stimuli (stimulus artifacts, lower panel) initiates monomorphic VT which persists after pacing ceases (PanelB).

FIG. 56 shows isochronal maps (2 msec isochrones) from a dog receiving the GFP adenovirus (sham #1454) and another receiving the SkM1/GFP adenovirus (#1448), per protocols in the Methods. Pacing was performed in the epicardial border zone in the middle of the infarct.

FIG. 57 shows the effect of SKM1 adenoviral infection of epicardial site. Dog was infarcted and injected 5 d before this experiment. Panel A: Photo of LV endocardial surface. Each panel is ECG (upper) and EG (lower). Broken line demarcates infarcted (lower) from non-infarcted (upper) region. Thin black lines mark EG recording sites. Note the EG in non-injected infarcted zone 2 is markedly fragmented. Infarcted zone 1 (injected with SKM1) shows a normal EG as do non-infarcted sites 3 and 4. Panel B: H and E stain of tissues from zones 2 (no SKM1) and 1 (+SKM1) show infarcted myocardium (×200). Inset in 1 is GFP positive; that in 2 is GFP negative (×400). Action potential in 1 has higher Vmax and amplitude than that from 2. Panel C: left: multiple impalements from SKM1-injected (red) and non-injected (black) zones show higher Vmaxin the former (P<0.05). The same is true for membrane responsiveness curves in both zones (panel C, right, P<0.05).

FIG. 58 shows the use of 100 nM TTX to discriminate SKM1 skeletal muscle Na channels from SCN5A cardiac Na channels in canine infarct 5d after SkM1/GFP adenoviral injection per FIG. 57. Panel A: multiple impalements in SKM1-injected vs. non-injected epicardial border zonebefore and after TTX. Panel B: Membrane responsiveness curves in the same 2 regions before and after TTX. Panel C: Membrane responsiveness from epicardial border zone of region injected with GFP adenovirus (no SkM1) versus non-injected. Results are X+/−SE for 5-40 impalements in panel A and 4-6 cells for each membrane responsiveness curve in Panels B and C. * indicates P<0.05 vs. other curve (2-way ANOVA).

FIG. 59 shows CV at 1 Hz pacing in normal and high (10.4 mM) K. Myocytes were cultured alone (CM), with GFP expressing HEK cells (CM+HEK) or with SKM1 expressing HEK cells (CM+HEK+SkM). In the mixed cultures the cell ratio was CM:HEK=9:1 at the time of experiment.

FIG. 60 demonstrates the use of stem cell to carry SkM1 to the 5-day infarct. The heavy line marks the upper margin of the infarct. Panel A shows ECGs and EGs from 4 representative sites. Site 5 received 700,000 hMSCs loaded with quantum dots and SkM1. Site 8: a representative infarcted region that received no MSCs. Site 1 is outside the infarct and Site 9 is outside but at the edge. Note that both Sites 5 and 8 have EGs that are narrow. Panel B: microelectrode maps of sites 5 and 8 demonstrating that at K+=4 mM the isochrones (5 ms) are comparable and that they do not become more tightly packed at 7 mM. Panel C: the relationship of Vmax to membrane potential for 4 infarcted animals that received a GFP virus in comparison to Sites 5 and 8 (39-41 impalements/site). Site 5 has the highest curve and both Sites 5 and 8 differ from the GFP curve. Panel D: conduction velocity at K=4 and 10 mM: At Site 5 at both [K+] propagation is faster than at Site 8; velocity does not decrease as [K+] increases.

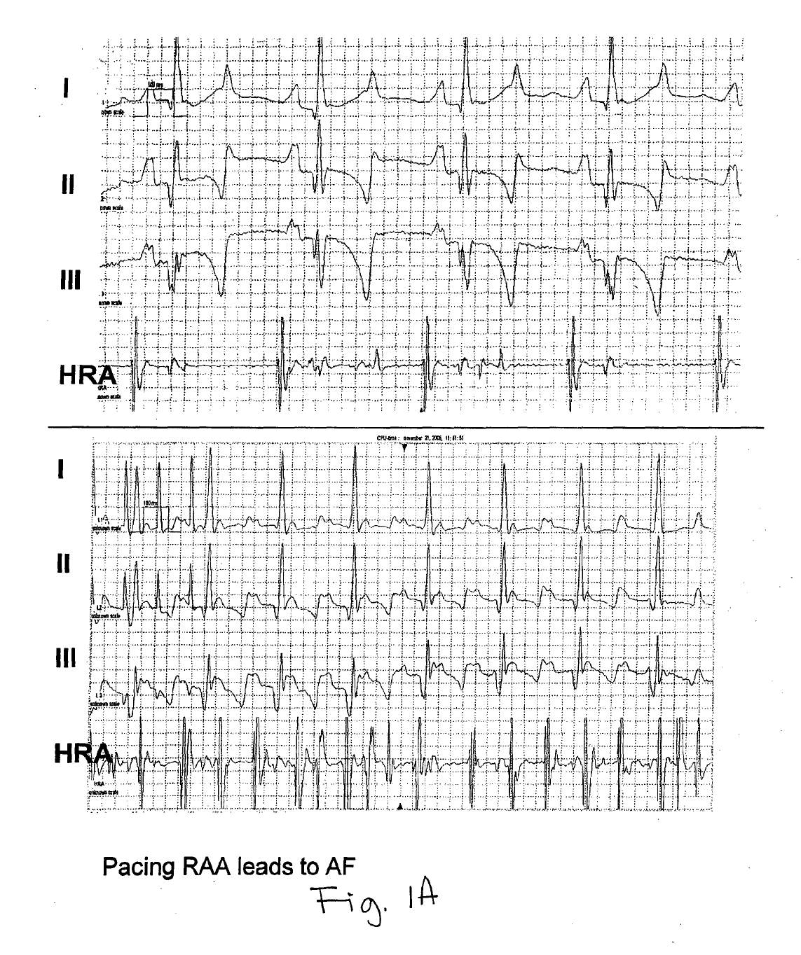

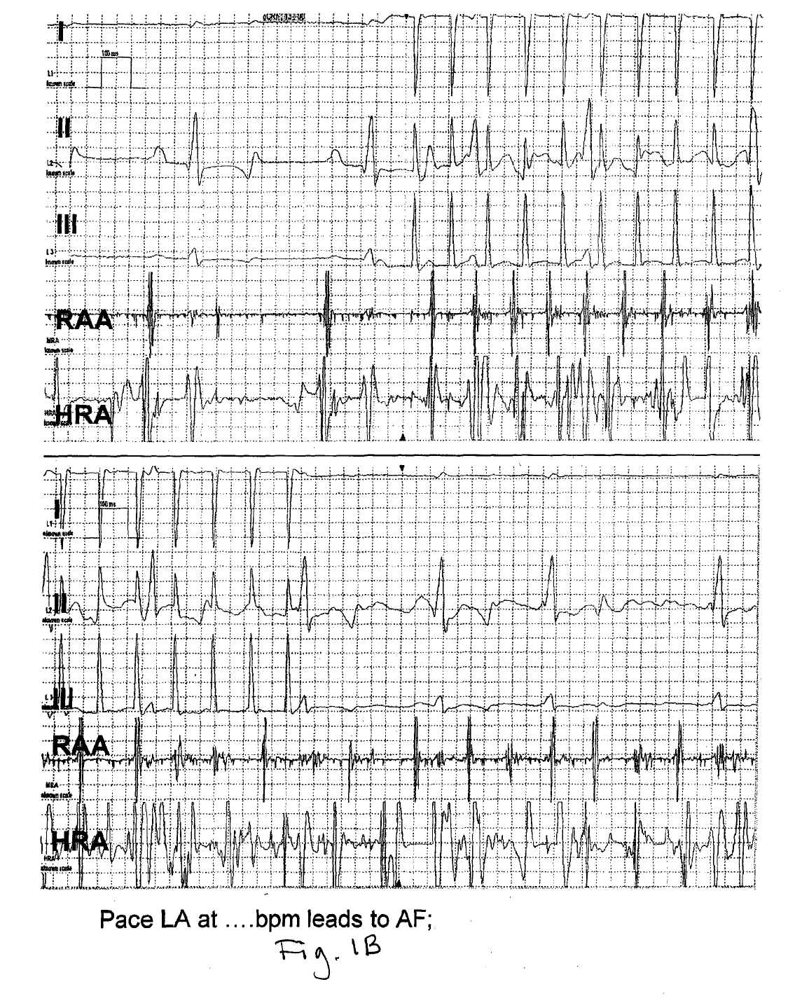

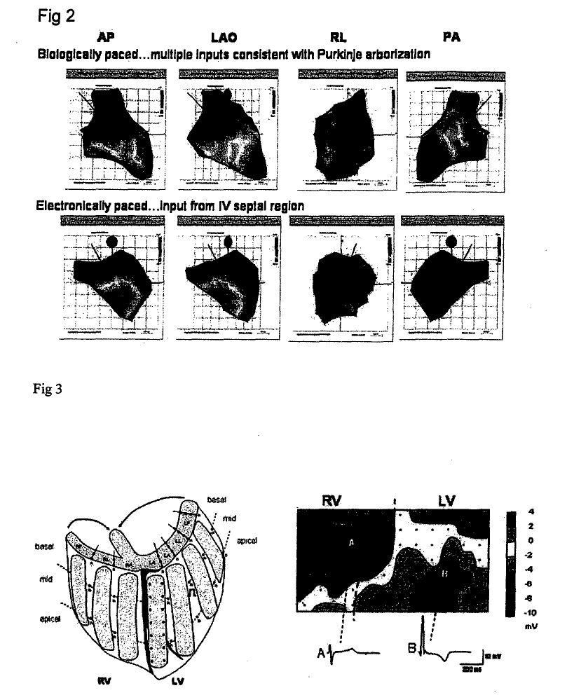

### DETAILED DESCRIPTION

Cardiac arrhythmias can result from some combination of: 1) abnormal impulse initiation such that the cardiac impulse originates in a location other than the primary pacemaker, the sinoatrial node and/or via mechanisms other than the normal pacemaker potential; 2) abnormal conduction; and 3) combinations of 1 and 2. As an example, in the presence of abnormal conduction, the activating waveform might proceed along a well defined anatomic pathway and reach its point of origin after the standing wave that is the action potential has ended and the tissue is no longer refractory (Noble, D. (1979), Initiation of the Heart Beat, Second edition, Clarendon Press, Oxford, 186 pp). For this to occur, a number of specific changes from normal conduction must exist. First, somewhere within the affected pathway, there must be conduction block in one direction while conduction in the other direction must be sufficiently slow to reach the point of origin after it is no longer refractory. The focus of the present invention is on this second arrhythmia mechanism as both atrial fibrillation and ventricular tachycardia involve this reentrant excitation.

Pharmacotherapy for cardiac arrhythmias has been hampered by a number of issues. First, since many arrhythmias result from pathology that involves loss of function, it would be logical to want to restore function to normal. Yet, there are far more blocking agents for ion channels available than there are activators. Second, pharmacotherapy is limited in scope to modifying channels that are already resident in the myocardium. However, there are alternative channels that might have favorable impact on cardiac rhythm that exist in other tissues or can be made by mutagenesis. Third, it is rare for a pharmacologic agent to be highly selective. Agents that alter Na channels also alter K or Ca channels leading to unwanted “side effects” including proarrhythmia. Recently, the feasibility of delivering genes via adenoviruses or cells transfected with specific channel genes to the “in vivo” myocardium (Miake, J., et al. (2002), Nature 419, 132-133; Qu, J., et al. (2003), Circulation 107, 1106-1109; Plotnikov, A. N., et al., (2004), Circulation 109, 506-512) has been demonstrated. Whether using viral gene delivery or transfected cells (which integrate into the cardiac syncytium by forming gap junctions) a spontaneous rhythm was generated by delivering HCN2, a pacemaker channel gene to the in vivo canine ventricle. (Qu, J., et al. (2003), Circulation 107, 1106-1109; Plotnikov, A. N., et al., (2004), Circulation 109, 506-512; Potapova, I., et al., (2004), Circ. Res. 94, 952-959). In a separate set of studies, it was also demonstrated that small interfering RNA (siRNA) can transfer from one cell to another via gap junctions and initiate gene silencing in the target cell (Valiunas, V., et al., (2005), J. Physiol. 568.2, 459-468).

Since conduction abnormalities often involve reduced conduction velocity in a segment of the reentrant pathway, a desirable approach to therapy might be to enhance sodium current. Unfortunately, this is not easily achieved. Often some region of the reentrant pathway is depolarized (Janse, M. J. et al. (1989), Physiological Reviews 69, 1049-1169) leading to steady state inactivation of sodium channels. Currently available antiarrhythmic drugs focused on the sodium channel tend to shift inactivation in the negative direction, creating more steady state inactivation (Scheuer, T. (1999), J. Gen. Physiol. 113, 3-6). This approach would further slow conduction and could convert unidirectional to bidirectional block. The latter would then be expected to terminate conduction in the reentrant pathway. Unfortunately, such drugs can also slow conduction in other pathways setting up reentrant proarrhythmia where no arrhythmia previously existed.

Recently the feasibility of delivering ion channel genes in vivo via viral constructs or cells has been demonstrated (Qu, J., et al. (2003), Circulation 107, 1106-1109; Plotnikov, A. N., et al., (2004), Circulation 109, 506-512; Potapova, I., et al., (2004), Circ. Res. 94, 952-959. The mHCN2 gene was delivered to the canine atrium or conducting system in an adenovirus, or in transfected human mesenchymal stem cells (hMSCs) to the canine left ventricular free wall. In each case the gene delivery resulted in the genesis of a spontaneous rhythm. Using a cell therapy approach, the mHCN2 gene was transfected into hMSCs, which expressed a large I_{f}-like pacemaker current (Potapova, I., et al., (2004), Circ. Res. 94, 952-959). The hMSCs also natively expressed the cardiac connexins 40 and 43, and formed functional gap junctions electrically integrating the hMSCs into the canine ventricular syncytium (Valiunas, V. et al., (2004), J. Physiol. 555, 617-626). The success of these initial studies (Miake, J., et al. (2002), Nature 419, 132-133; Qu, J., et al. (2003), Circulation 107, 1106-1109; Plotnikov, A. N., et al., (2004), Circulation 109, 506-512; Potapova, I., et al., (2004), Circ. Res. 94, 952-959; Kehat, I., et al., (2004), Nat. Biotechnol. 22, 1282-1289; Xue, T., et al., (2005), Circulation 111, 11-20) has stimulated the central theories behind the present invention: gene and cell therapy can be successfully used to terminate arrhythmias.

Given the feasibility of such approaches what are the potential advantages? First, gene and cell therapies unlike pharmacologic therapy are not limited by the channels and transporters expressed by the native cardiac myocytes. Instead, channels resident in other tissues or man-made mutant or chimeric channels with more favorable biophysical properties can be employed (Bucchi, A., et al., (2006), Circulation 114, 992-999). Second, if a favorable therapy involves blocking particular resident ion channel subunits then as will be described below, specific silencing constructs (the genes encoding small interfering RNAs) can be delivered by viral constructs or transfected into the delivery cell type (Valiunas, V., et al., (2005), J. Physiol. 568.2, 459-468). This unique arsenal of antiarrhythmic tools allows for the first time a “rational” approach to antiarrhythmic therapy in which the biophysical properties of an ideal therapeutic agent are defined, synthesized and delivered.

In general, methods of the present invention relate to one mechanism, reentry, and two arrhythmias—ischemia-associated ventricular tachycardia (VT) and atrial tachycardia-induced atrial fibrillation (AF). Both of these arrhythmias are usually reentrant and a great deal is known regarding mechanisms underlying reentry. By extension the therapy described would apply to any and all other reentrant arrhythmias as well. The present invention relates to the use of gene therapy to treat these conditions because it provides an opportunity to alter the machinery of the myocyte itself. Cell therapy is used not to construct new myocytes or repair/regenerate myocardium (a voluminous field in its own right) but rather as a platform to transmit specific genetic information to the myocytes in an arrhythmogenic setting.

Accordingly, the present invention provides gene therapy and cell therapy approaches to modify conduction in ways that prevent completion of reentrant circuits and therefore short-circuit expression of the arrhythmias. It is believed that gene and cell therapies are superior to drugs and promise to be less invasive and intrusive on life-style than devices in preventing and treating these arrhythmias.

Embodiments of the present invention operate from the knowledge that most VTs are reentrant, and are initiated either by abnormal conduction of a sinus beat or by an initial reentrant, triggered or automatic beat. There is also considerable evidence that most AF is reentrant, although initiation in many individuals with otherwise normal hearts or with structural heart disease can result from triggering foci in the pulmonary veins and elsewhere.

Methods of the present invention are based on the concept that reentry can be prevented/suppressed by speeding conduction or by blocking conduction and/or by prolonging ERP. While this hypothesis has long been recognized and in part tested experimentally and clinically using drugs and ablation techniques, method of the present invention are novel and use the following strategies, which are not accessible pharmacologically or with devices.

AF can be delayed in its evolution from paroxysmal to persistent by modifying the conducting properties of the tissues in which the impulses are propagating. Modifying the conducting properties of the tissues can be brought about with two models: (1) long-term atrial tachy-pacing in which improving gap junctional conductance may bring advantage by improving cell-cell communication, and (2) rapidly firing foci (induced by pacing) in the LA appendage in which conduction block may be advantageous in preventing propagation of the triggering beats to the body of the atrium. Accordingly the present invention relates to uniformly improving conduction or prolonging refractoriness throughout the atria or regionally depressing conduction in settings where a local, triggered focus is initiating fibrillation to delay onset of or prevent propagation of the arrhythmia.

VT can be delayed in onset or prevented from evolving by delivering Na or K channel genes to alter conduction and/or refractoriness. Accordingly, methods of the present invention provide an increase in conduction velocity in areas of slow conduction or such that impulses will encounter refractory tissues and be unable to propagate further, or else to suppress conduction completely. In other embodiments of the present invention, there is provided methods to prolong refractoriness as an alternative or complementary approach in treating AF or VT.

The present invention thus provides novel therapies for treating AF and/or VT that accelerate conduction by: (a) overexpressing Cx46 and/or (b) overexpressing skeletal muscle Na channels which activate fully at depolarized potentials and/or (c) using ERG3 to hyperpolarize the membrane such that reentrant impulses reach zones of tissue that are still refractory. In other embodiments, conduction is blocked through the use Cx31.9 or a NaV1.5 alpha subunit mRNA. Yet, in another model, refractoriness is prolonged without altering APD such that reentry impulses are blocked but the proarrhythmia problem of QT prolongation is avoided, through the use of HERG1 or MiRP1 siRNA. Methods of the present invention are based on known mechanisms that have been reaffirmed during a century of study of reentry and yet the methods speak to approaches never before accessible. See Table 1 for a summary of the intervention strategies, grouped together with respect to their mechanistic outcome.

TABLE 1

| | |
|---|---|
| I. | Accelerate conduction |

| | |
|---|---|
| A. | Overexpress endogenous connexins (Cx 40, Cx43, Cx45) |

| | |
|---|---|
| 1. | Chemical chaperone |
| 2. | MMP-7 inhibition |

| | |
|---|---|
| B. | Overexpress exogenous connexins |

| | |
|---|---|
| 1. | Cx 32, |
| 2. | Cx46 |

| | |
|---|---|
| C. | Increase availability Of I_{Na} (SKM1 channel (SCN4A) or mutant |
| | SKM1 channel (SCN4a-G1306E) |
| D. | Increase membrane potential |

| | |
|---|---|
| 1. | ERG3 |
| 2. | Kir2.1 |
| 3. | Kir2.2 |

| | |
|---|---|
| II. | Slow or block conduction |

| | |
|---|---|
| A. | Overexpress Cx31.9 |
| B. | Nav1.5 alpha subunit siRNA |

| | |
|---|---|
| III. | Increase ERP without altering APD |

| | |
|---|---|
| A. | Atrium |

| | |
|---|---|
| 1. | hERG mutant (slowed deactivation kinetics)(K538) |
| 2. | MiRP1 siRNA |

| | |
|---|---|
| B. | Ventricle |

| | |
|---|---|
| 1. | hERG1 without MiRP1 |
| 2. | hERG1 mutant having slowed deactivation kinetics |
| 3. | MiRP1 siRNA |

With regard to therapies under I. in Table 1, each intervention may be used independently of one another or used together. They may be administered using viral vectors or hMSCs, and can be administered regionally to a chamber that has depressed conduction or, if mapping shows localized conduction depression, then to that local site.

With regard to therapies under II in Table 1, these approaches are a variation on what is attempted with drugs or with ablation. Preferably the therapies are delivered locally (in a sense a gene- or cell-therapy ablation) and employ Cx31.9 (the human variant of the murine Cx30.2) to induce conduction block at a local site without killing tissue, as occurs with ablation. In another embodiment siRNA to the Nav1.5 alpha subunit is employed.

With regard to therapies under III in Table 1, the underlying strategies have been attempted with drugs (e.g. flecainide). However, these drugs also depress conduction in a fashion that can be deleterious. In the atria, the present invention preferably provides therapies as either chamber-wide, using a viral vector, or locally, using virus or an hMSC platform. In the infarcted ventricle, the present invention provides preferably a local therapy using the hMSC platform. Among the constructs for lengthening ERP without affecting APD are an HERG1 mutant having slowed deactivation kinetics (K538A or L539W), MiRP1 siRNA which by subtracting the MiRP1 subunit will prolong repolarization by slowing native ERG kinetics, or administering hMSCs carrying ERG without the MiRP1 subunit.

### Accelerating Conduction

### Modulating Connexin Expression as an Antiarrhythmic Intervention

One embodiment of the present invention relates to the use of connexins in the treatment of arrhythmias. Normal conduction velocity is maintained, in part, by the appropriate localization and function of gap junctions in the myocardium. Gap junction channels in vertebrates are formed from subunit proteins called connexins. Each gap junction channel is composed of two hemichannels each of which contains 6 connexins. When two cells are in close apposition it is possible for a hemichannel from each cell to link together via the extracellular loops of the component connexins to form gap junction channels. This channel represents a unique intercellular pathway because it is the only form of intercellular communication that excludes the extracellular space. For reasons that are not completely understood, but have been attributed to lipid membrane domains including lipid rafts, (Locke and Harris, 2005) gap junction channels tend to aggregate and form plaques containing tens to thousands of channels (Goodenough, 1975).

Connexin43 (Cx43), Connexin40 (Cx40), and connexin45 (Cx45) are major connexins expressed in human heart (Severs et al., 2006). The gap junction channels formed by these connexins conduct monovalent ions efficiently, generating the local circuit currents necessary for propagation. Cx43 is expressed in the ventricles, atrium and Purkinje fibers while Cx40 is expressed in the SA node, atrium, bundle branches and Purkinje fibers. Cx45 is expressed in the SA and AV nodes and the conducting system (Van Veen et al., 2001; Beyer, 1993). More recently it has been shown that connexin 30.2 is expressed in the AV node of mice (Bukauskas et al., 2006) while Belluardo et al. (2001) have shown human 31.9 to be its ortholog and White et al., (2002) have determined its unitary conductance and gating properties and demonstrated that it is expressed within the human heart. However, its exact distribution is yet to be determined. Often coexpression within any one cell gives rise to gap junction channels composed of two or more connexins. These channel types are referred to as heteromeric. The cardiac connexins have been shown to form heteromeric channels (Brink et al., 1997; Valiunas et al., 2002; Cottrell et al., 2002).

Action potential propagation within the heart gap junctions play a critical role in the propagation of action potentials. The classic studies by Barr et al. (1965) and Weidmann (1966; 1970) were the first to establish that currents associated with action potential propagation move from myocyte to myocyte via gap junctions and that reduction in gap junction-mediated communication resulted in propagation failure. A number of subsequent studies have detailed the critical role gap junctions play in the propagation of cardiac action potentials (Verheijck et al., 1998; Wilders et al., 1996; Brink et al., 1996; Cole et al., 1988; deGroot et a1.2003: Akar et al., 2003; Tan and Joyner, 1990). An important factor in the conduction of the cardiac action potential is longitudinal resistance arising from the cytoplasm and gap junctional membranes connecting cellular interiors. In fact conduction velocity, 0, is inversely proportional to the square root of the longitudinal resistance, Ri, (θα1/√Ri). Ri is composed of both cytoplasmic resistance and junctional membrane resistance such that Ri=Rcyto+Rj. In a typical ventricular cardiac myocyte that is 80 um in length and 10-15 um in width the total longitudinal resistance contributed by the cytoplasm is ˜2MΩ or 500 nS assuming the resistivity of the cytoplasm to be 300-400 Ω-cm where as the junctional resistance at the intercalated disc is ˜10MΩ or 100 nS.

(Brink et al. 1996). These values make it clear that junctional resistance or conductance at the intercalated disc is the dominant determinant of longitudinal resistance. Using acutely isolated myocytes a number of investigators have shown that reduction in junctional conductance slows conduction and can be a major determinant in conduction failure (Cole et al., 1988; Tan and Joyner, 1990; Joyner et al., 1991; de Groot et al., 2003). A similar conclusion has been drawn from optical mapping of a perfused ventricular wedge preparation (Akar et al., 2003; Poelzing and Rosenbaum, 2004). Accordingly, since reduction in junctional conductance results in conduction failure, the present inventors believe that increasing the number of functional gap junctional channels will sufficiently increase action potential conduction velocity to act as a therapy for arrhythmias.

Thus, one method for treating re-entrant arrhythmias relates to a reduction in longitudinal resistance which would stop reentrance. Arrhythmias arising from re-entry are a result of inhomogeneity in repolarization within a local region of the atrial or ventricular myocardium. FIG. 30 illustrates schematically how a re-entrant arrhythmia might arise and how alteration of longitudinal resistance could abolish such an arrhythmia. FIG. 30A shows two conducting limbs resulting from an ischemic episode or some equivalent where the right-hand limb experiences slowed conduction and conduction failure as indicated by the box and stop sign due to membrane depolarization in the area above the region of conduction block. The left-hand limb action potential is then able to conduct in the retrograde direction and has sufficient strength to propagate through the region of conduction failure. This results in a local re-entrant circuit conducting faster than the pacing rate of the heart where the retrograde action potential occurs after the absolute refractory period of the action potential generated in the left-hand limb. If conduction velocity within the left-hand limb is enhanced by increasing the junctional conductance between cells in this adversely affected region of myocardium, the retrograde re-entrant circuit delivers the “second” action potential during the absolute refractory period of the first, thereby abolishing the re-entrant action potential and any undesirable effects the re-entrant arrhythmia might have been causing. This latter case is shown in FIG. 30C.

Another example illustrates the importance of connexins and gap junctions in the modulation of cardiac rhythm and arrhythmias. Adult human mesenchymal stem cells (hMSCs) that express HCN2 delivered in vivo to the heart can provide a stable pacemaker (Potopova et al., 2004; Valiunas et al., 2004) because the hMSCs couple electrically with myocytes via Cx43 and Cx40. Moreover, a number of recent studies have shown the importance of connexins and hence gap junctions in arrhythmias and infarcts. For example, over-expression of Cx45 results in ventricular tachycardia in mice (Betsuyaku et al., 2006; 2004) while mutations of Cx40 are associated with atrial fibrillation in humans (Gollob et al., 2006: Saffitz, 2006). The mouse connexin Cx30.2 (the ortholog of human Cx31.9) has recently been shown to slow conduction in the murine AV node (Kreuzberg et al., 2006). Arrhythmias arising from border zones of infarcts equate with redistribution of Cx43 that correlate with reentrant arrhythmia (Peters et al., 1997). In addition, the matrix metalloproteinase (MMP-7) has been shown to have a direct association with Cx43 in mice with experimentally-induced myocardial infarctions. This association is absent in MMP-7 knock out mice. (Lindsey et al., 2006).

### Overexpression of Endogenous Connexins (Cx40, Cx43 and Cx45)

One embodiment of the present invention provides for a method of overexpressing heart tissue endogenous connexins. There is provided a method of treating atrial fibrillation comprising modifying the conducting properties of the tissues in which the impulses are propagating, the method comprises improving conduction in the tissues by increasing gap junctional conductance in said tissues. Overexpressing connexins, and thus increasing levels of the connexins in the heart provides an anti-arrhythmic effect. In certain embodiments, gap junctional conductance is preferably increased globally throughout the entire atrium and other embodiments, conductance is increased locally by administering constructs that provide expression of connexins. In certain embodiments, hMSCs transfected with endogenous heart connexins are delivered globally or locally. As noted above, connexins found in the heart (endogenous) are Cx40, Cx43 or Cx45. In certain embodiments, viral vectors capable of expressing endogenous connexins are delivered globally or locally to the desired area of the atrium. Any suitable viral vector may be used, but preferably lentiviral vectors are employed. Other suitable means to affect overexpression of endogenous connexins may be used. In certain embodiments, chemical chaperons are used to mediate overexpression.

### Chemical Chaperone Mediated Overexpression

4PB is an FDA approved drug that can be administered orally, which is being tested for effectiveness in spinal muscular atrophy (Wirth et al., 2006; Brahe et al., 2005), and cystic fibrosis (Singh et al., 2006). Zp123 has been shown to affect arrhythmias in animal models (Eloff et al., 2003). In addition, stress in the form of mildly elevated temperatures up-regulates connexin expression and increases the total number of functional channels (VanSlyke and Musil, 2005). The chemical chaperone (4PB), a known inhibitor of histone deacetylase, an enzyme associated with inactivation of gene transcription, increases Cx43 production at concentrations in the mM range and exposure times of hours. Not only is Cx43 production elevated 2-5 fold but enhanced dye transfer is observed (Asklund et al., 2004). Accordingly, one embodiment of the present invention provides the use of 4PB to increase the rate of coupling between hMSCs and myocytes (and hence shorten the coupling time between hMSCs and myocytes with exposure to 4PB). The present inventors have determined that the time course of coupling between hMSCs or model cells with myocytes reaches 50% of maximum within 48 hours. Further, the present inventors have shown that 4PB up-regulates Cx43 in HeLa cells transfected with Cx43.

Antiarrhythmic peptides (AAP) such as the synthetic antiarrhythmic hexapeptide zp123 not only prevent spontaneous ventricular arrhythmias (Hennen et al., 2006) but also increase cell to cell coupling mediated by Cx43 (Clarke et al, 2006; Guerra et al., 2006) with exposure times of 1-5 hours and at nanomolar concentrations. In addition, zp123 enhances conduction within the heart (Eloff et al., Circ. 2003). Another feature of zp123 is its prolonged half life relative to other antiarrhythmic peptides such as AAP10 (the half life of zp123 is approximately 10 days while that of AAP10 is 10 minutes (Kjolbye et al., 2003)). Further, Stahlut et al., (2006, Cell Comm and Adh. 13:21-27) recently showed that zp123 increases Cx43 levels with 24 hour exposures at a concentration of 100 nM. The molecular mechanism by which zp123 acts has not been elucidated but it does not appear to affect the duration or shape of the cardiac action potential nor does it affect I_{Na} (Eloff et al., 2003). Consequently, one embodiment of the present invention provides the use of zp123 to upregulate Cx43 protein levels to shorten the time course of coupling between hMSC and myocyte. In another embodiment, increasing gap junctional conductance comprises administering a chemical stimulator of connexin expression to desired atrial tissues to cause said tissues to overexpress endogenous connexins selected from the group consisting of Cx 40, 43, 45. Preferred chemical stimulators are 4PB or Zp123.

### MMP-7 Inhibition

Matrix metalloproteinase (MMP-7) activity is associated with ischemia and infarction as well as slowed cardiac action potential propagation. Lindsey et al., (2006) have shown the message levels for Cx43 are unaffected by MMP-7 activity in control and in MMP-7 knockout mice. But their data also show that the elevated activity of MMP-7 triggered by infarction results in the cleavage of the C-terminus of Cx43. Finally, their data suggest that the total abundance of Cx43 is reduced by 53% with induced infarcts in wild type mice but is unaltered in MMP-7 knockout mice. The drug Gefitinib, used in lung cancer, inhibits MMP-7 activity via inhibition of tyrosine kinase (Mimori et al., 2004) at clinically relevant dosages.

Another recently appreciated feature of infracted regions of the heart is the role of MMP-7 has and its interactions with Cx43. Ischemia appears to trigger interactions or enhance interactions between MMP-7 and Cx43 that result in reduced coupling in wild type mice.

Interestingly MMP-7 knockout mice have been shown to be resistant. Accordingly, it is believed that inhibition of MMP-7 would enhance cell to cell coupling in myocytes and thus increase gsp junctional conductance.

The present invention thus provides a method of treating atrial fibrillation comprising modifying the conducting properties of the tissues in which the impulses are propagating, the method comprising improving conduction in the tissues by increasing gap junctional conductance in said tissues. In certain embodiments, increasing gap junctional conductance comprises administering a MMP-7 inhibitor to the tissues to inhibit MMP-7 and thus cause overexpression endogenous connexins. A preferred MMP-7 inhibitor is Gefitinib.

### Adhesion Molecule Mediated Overexpression

Among the ways to up-regulate connexin expression are increasing expression of adhesion molecules such as alpha and beta catenin and cadherin (Jongen et al., 1991: Prowse et al., 1997: Wei et al., 2005), and chemical stimulation with 4-phenylbutyrate (4PB) (Asklund et al., 2004) or the synthetic antiarrhythmic peptide zp123 (Axelsen et al., 2006). In cells that already make sufficient amounts of catenins and cadherins (e.g. hMSCs) augmentation of expression is not as effective as it is with cells that are deficient in catenin and/or cadherin expression. HMSCs make both alpha and beta catenins and express both E and N cadherins. Cardiac myocytes express catenins and N-cadherins as well. Interestingly if N-cadherin is over-expressed in cardiac myocytes cardiac myopathies occur (Li et al., 2006). Thus, one embodiment of the present invention relates to the use of adhesion molecules as a tool to promote and increase the number of functional gap junction channels in hMSCs as an alternative approach to chemical chaperones (discussed above).

### Exogenous Connexins—Cx46

Modulating expression of selective gap junctional proteins will alter conduction due to individual single channel conductance properties and the corresponding effect on cell coupling. A global increase in gap junctional conductance, can provide a beneficial therapy for AF. Another embodiment of the present invention provides globally increasing gap junctional conductance by overexpressing Cx46 in the atrium to provide improved inter-atrial conduction and delay AF onset and/or slow its evolution into a persistent state. In a preferred embodiment, an AAV or another viral construct (such as a construct derived from a lentivirus) expressing Cx46 is delivered globally to the atria. In other embodiments hMSCs having been transfected to express Cx 46 may be delivered globally or locally to desired portions of the atria.

In another embodiment, pharmacological stimulation of hMSCs that have been transfected with exogenous connexin (Cx46) is used to shorten effective coupling time between hMSCs and myocytes.

Cell coupling will be increased by adenovirus or adeno-associated virus over-expression of large conductance Cx46 isoform in myocytes and/or expression in co-cultured hMSCs. Over-expression will be accomplished directly in the myocytes by viral delivery, and in the case of Cx46 also in coupled hMSCs co-cultured with the myocytes. Cx46 has pH sensitivity similar to Cx43/40 and can form heteromeric and heterotypic channels with them, but is a large conductance channel that may provide an additional measure of conductance under conditions where conductance would otherwise be compromised.

### Exogenous Connexin Cx32

Modulating expression of selective gap junctional proteins will alter conduction due to individual single channel conductance properties and the corresponding effect on cell coupling. A global increase in gap junctional conductance, particularly of a pH resistant connexin isoform, Cx32 can provide a beneficial therapy for VT. Gene and cell based connexin over-expression increases conduction in a cell culture model and protect against conduction effects of acidosis. The effect of both global over-expression and regional over-expression (in a culture area that bridges regions of normal myocytes) will be studied. Over-expression will be accomplished directly in the myocytes by viral delivery. Cx32 is only weakly pH sensitive and does not form heteromeric channels with cardiac connexins. Cx32 expression may therefore better preserve conductance under acidic conditions.

Ischemia is one cause of arrhythmias. One feature of ischemia is the acidification of myocytes (Duffy et al., 2004; Casio et al., 2005). The linkage between ischemia and connexins arises because gap junction channels are pH sensitive (Morley et al., 1997; Bukauskus and Verselis 2000). Acidification of the cytoplasm causes gap junction channels to close, with one notable exception, Cx32 (Morely et al., 1996; Liu et al., 1993), which is the least pH sensitive connexin thus far documented. Ischemia induced uncoupling of myocytes is a potential contributor to reentrant arrhythmias in regions such as the boarder zone of an infarct. Thus, one result of acidosis induced by ischemia is the reduction in gap junction mediated cell to cell communication.

Cx32 is the least pH sensitive connexins, pKa=6.2. In addition, it does not form heteromeric channels with Cx43. Successful transfection of Cx32 will generate myocyte cell pairs resistant to pH induced uncoupling. In addition, because Cx32 does not readily form heteromeric channels with Cx43 (Das Sarma et al., 2001) then once transfected into myocytes they represent an independent population of channels acting without influence from interactions with endogenous connexins. The notion of Cx32 functioning within the heart is not novel. In fact Plum et al., (2000) generated a mouse in which Cx43 was replaced with Cx32. The latter rescued the Cx43 deficient mice that would otherwise would have died at birth. One potential concern with regard to Cx32 is that cellular acidification during an ischemic episode or within a resultant infarct is that Cx32 will allow more readily transfer of H+ from myocyte to myocyte because of its ability to remain in the open state with acidification. In fact, Cx32 has more restrictive permeability characteristics than Cx43 (Goldberg et al., 2004). This fact along with the observation that H+ transit through gap junction channels composed of Cx43 is accomplished by “proton-porter” molecules (Zoniboni et al., 2003; Vaughan-Jones et al., 2006) and not by direct permeation of the H+ argue that Cx32 would not compromise functioning myocytes within or on the fringes of an infarct or any ischemic tissue in terms of the spreading of H+.

The present invention thus provides a method of treating atrial fibrillation by increasing gap junctional conductance by delivering to the atrium or portions thereof, hMSCs transfected with Cx32. In other embodiments, a viral vector capable of expressing Cx32 is utilized. A preferred viral vector is derived from a lentivirus.

### Accelerating Conduction—Increasing Availability of I_{Na}

Conduction can be enhanced (and normalized) in depolarized cells in a reentrant pathway by altering Na channel availability such that a greater number of sodium channels can be activated from the depolarized membrane potential. This change in biophysical properties can be elicited using hMSCs or a viral vector in either of two ways; first, by delivering hMSCs containing Na channels with a depolarized inactivation-voltage curve. Two preferred Na channels include: the skeletal muscle Na channel (activation midpoint −68 mV)(SCN4a) and/or a specific mutant of that channel (SCN4a-G1306E)(activation midpoint −57 mV).

The alpha subunit of the sodium channel encodes four domains (I-IV) each of which contains six transmembrane spanning regions (S1-S6). The pore is located between transmembrane domains S5 and S6, while the voltage sensor is located in the S4 transmembrane domain (Catterall, W. A. (2000), Neuron 26, 13-25). At least 10 different sodium channel genes encode alpha subunits in the mammalian genome and these have been cloned from brain, spinal cord, skeletal and cardiac muscle, uterus, and glia (Lopreato, G. F., et al., (2001), PNAS 98, 7588-7592). Besides the native channels, a number of mutations have been identified that produce diseases of muscle, nerve and heart (Ashcroft, F. M. (2000), Ion Channels and Disease, Academic Press, San Diego, Calif., 481 pp).

Since slow conduction is an essential feature of reentrant cardiac arrhythmias, it seems worth considering other mammalian sodium channels that might have more favorable properties than the cardiac Na channel in circumstances that favor slow conduction. One such circumstance is membrane depolarization and so the voltage dependence of steady state inactivation is of interest. Cohen Table I provides a comparison of the midpoints for steady state inactivation and slope factor for the cardiac and skeletal muscle sodium channels and a specific mutant of the skeletal muscle channel (G1306E). Clearly there is a wide range for steady state inactivation with the cardiac sodium channel (SCN5A) being relatively negative (−95 mV with slope factor −6.5 mV) (Fozzard, H. A. et al., (1996), Physiol. Rev. 76, 887-926) while the skeletal muscle sodium channel Skm1 is more positive (−68 mV with slope factor −5.4 mV). Thus, if cardiac muscle is depolarized to −65 mV, virtually all cardiac sodium channels would be inactivated, while a similar depolarization would leave almost half of normal skeletal muscle sodium channels available. This observation thus relates to a method of the present invention that the use of alternative sodium channels with more favorable biophysical properties serve as a useful antiarrhythmic therapy.

Besides existing sodium channels, much has been learned from structure-function studies in which specific amino acids have been mutated. An extensive study of the S4 domain of the skeletal muscle sodium channel was conducted by Haywood et al. (1996), J. Gen. Physiol. 107, 559-576 in an attempt to understand the origins of enhanced excitability in inherited human myotonia. It was demonstrated that steady state inactivation was shifted more positive to −57 mV in one these mutant channels (G1306E) (see Table 2). For the same depolarization to −65 mV more than 70% of these mutant sodium channels would be available.

TABLE 2
Sodium Channel Inactivation

| | V ½ (mV) | Slope factor (mV) |
|---|---|---|
| *Cardiac Na Channel | −95 | −6.5 |
| (SCN5a) | | |
| SkM1 | −68 | −5.4 |
| skM1(G1306E) | −57 | −5.2 |

*Note that the midpoint of inactivation for SCN5a approaches −80 mV for very brief (20 msec.) conditioning pulses.

Considerable ion channel remodeling occurs with both AF (Wijffels M C, Kirchhof C J, Dorland R, Allessie M A. Atrial fibrillation begets atrial fibrillation. A study in awake chronically instrumented goats. Circulation 1995; 92(7):1954-68; Morillo C A, Klein G J, Jones D L, Guiraudon C M. Chronic rapid atrial pacing. Structural, functional, and electrophysiological characteristics of a new model of sustained atrial fibrillation. Circulation 1995; 91(5):1588-95) and in the ventricle post-infarction (Pinto J M, Boyden P A. Electrical remodeling in ischemia and infarction. Cardiovasc Res 1999; 42(2):284-97; Wasson S, Reddy H K, Dohrmann M L. Current perspectives of electrical remodeling and its therapeutic implications. J Cardiovasc Pharmacol Ther 2004; 9(2):129-44). This remodeling can contribute to conduction abnormalities and arrhythmogenesis. The present invention relates not to reverse this remodeling, but rather to functionally substitute for any deficits so as to restore normal conduction. To this end, it is important to understand what ion channel dysfunctions are present in the diseased heart, and in particular those dysfunctions that directly relate to propagation.

In the case of AF, I_{CaL}, and Iₜₒ are reduced by about 70%, without any alteration in their voltage- or time-dependence. These changes are due to a reduction in the number of functional channels in the membrane (Yue L, Feng J, Gaspo R, Li G R, Wang Z, Nattel S. Ionic remodelling underlying action potential changes in a canine model of atrial fibrillation. Circ Res 1997; 81(4):512-25). The use of pharmacological probes that mimic the effect of reduced I_{CaL} or Iₜₒ suggest that k_{CaL} plays a central role in APD alteration compared to Iₜₒ despite the quantitatively similar reduction. A reduction in Na⁺ current density was also found in dogs with AF but not in humans, while a reduction in I_{Kur} was found in humans but not in dogs. Alterations in intercellular electrical connections have been observed, particularly in connexin 43 and 40 proteins, with modifications varying in different species (Elvan A, Huang X D, Pressler M L, Zipes D P. Radiofrequency catheter ablation of the atria eliminates pacing-induced sustained atrial fibrillation and reduces connexin 43 in dogs. Circulation 1997; 96(5):1675-85; vvan der Velden H M, van Kempen M J, Wijffels M C, van Z M, Groenewegen W A, Allessie M A, Jongsma H J. Altered pattern of connexin40 distribution in persistent atrial fibrillation in the goat. J Cardiovasc Electrophysiol 1998; 9(6):596-607). AF also causes alteration in Ca²⁺ handling, including decreased SERCA protein, loss of sarcoplasmic reticulum and degeneration of contractile elements (Brundel BJ, van G, I, Henning R H, Tuinenburg A E, Deelman L E, Tieleman R G, Grandjean J G, van Gilst W H, Crijns H J. Gene expression of proteins influencing the calcium homeostasis in patients with persistent and paroxysmal atrial fibrillation. Cardiovasc Res 1999; 42(2):443-54; van Gelder I, Brundel B J, Henning R H, Tuinenburg A E, Tieleman R G, Deelman L, Grandjean J G, De Kam P J, van Gilst W H, Crijns H J. Alterations in gene expression of proteins involved in the calcium handling in patients with atrial fibrillation. J Cardiovasc Electrophysiol 1999; 10(4):552-60). The intracellular systolic Ca²⁺ transient is reduced in AF and the rates of Ca²⁺-release and reuptake are decreased. In AF, there is an extensive interstitial fibrosis associated with microreentry and fibrillatory conduction that may contribute to altered cell-cell interaction (Sun H, Gaspo R, Leblanc N, Nattel S. Cellular mechanisms of atrial contractile dysfunction caused by sustained atrial tachycardia. Circulation 1998; 98(7):719-27).

In the case of the healing ventricular infarct and conduction, significant K channel remodelling has been reported but the most relevant remodelling relates to depolarizing current (Na and to a lesser extent Ca channels) and gap junctional proteins. There is a marked reduction in I_{Na} and I_{Ca,L} density, and alteration of I_{Na} kinetics in the central common pathway of the re-entrant circuit (Baba S, Dun W, Cabo C, Boyden P A. Remodeling in cells from different regions of the reentrant circuit during ventricular tachycardia. Circulation 2005; 112(16):2386-96). There also is a reduction in transverse gap junctional conductance (Yao J A, Hussain W, Patel P, Peters N S, Boyden P A, Wit A L. Remodeling of gap junctional channel function in epicardial border zone of healing canine infarcts. Circ Res 2003; 92(4):437-43), consistent with earlier studies of slowed conduction in the transverse direction. While Cx43 distribution is altered in the epicardial border zone (Peters N S, Coromilas J, Severs N J, Wit A L. Disturbed connexin43 gap junction distribution correlates with the location of reentrant circuits in the epicardial border zone of healing canine infarcts that cause ventricular tachycardia. Circulation 1997; 95(4):988-96), the reduced transverse gap junctional coupling was found to be independent of any reduced Cx43 expression. These data indicate the channel remodelling is more complex than simply loss of functional proteins, and involves altered functionality of persisting channels. It is in part for this reason that we believe expression of non-native channels, with biophysical properties well suited to the diseased environment, represents an innovative and promising therapeutic approach.

The present invention relates to gene therapy approaches to target Na, K and Cx channels, based on existing knowledge of the ion channel remodeling that occurs following ischemia and during AF, and the understanding of the mechanisms underlying the arrhythmias that are observed in these settings. As stated above, Na channel function is known to be compromised in the post MI ventricle, with reduced current, altered kinetics, and altered channel distribution, and these abnormalities contribute to slow conduction and reentry arrhythmias. In addition, the reduced resting membrane potential found in regions of the post MI ventricle would further reduce any surviving Na current, exacerbating the problem. For the same reason, simply providing additional cardiac Na channels may be insufficient due to the possibility that expressed SCN5A channels will be inactivated in the depolarized milieu of the post MI heart. Therefore, one embodiment of the present invention relates to expressing an alternative Na channel isoform, in which the position of the steady-state inactivation curve is sufficiently positive to preserve function in the diseased heart. Exemplary Na channels include the skeletal muscle Na channel (SCN4a) and a well known mutant (SCN4a-G1306E).

Expression of a Na channel with relatively positive inactivation will enhance conduction in diseased, depolarized, tissue, providing a beneficial therapy for VT, especially reentry in ischemia/ventricular tachycardia. SCN4A (SKM1) is a preferred Na channel construct because it has a relatively positive inactivation. Another preferred Na channel is the SCN4A point mutation (SCN4A-G1306E) that exhibits more positive inactivation, in control and depolarized cultures.

Accordingly, one embodiment of the present invention provides a method of treating ventricular tachycardia by increasing conduction velocity in areas of slow conduction by altering Na channel availability to provide a greater number of Na channels to be activated from the depolarized membrane potential. In certain embodiments, the method utilizes an hMSC or a viral vector expressing a sodium channel having a more positive midpoint for steady state inactivation as compared to a normal heart Na channel. A preferred sodium channel a skeletal muscle sodium channel SCN4a or a mutant sodium channel such as SCN4a-1306E, which has even a more positive midpoint for steady state inactivation as compared to SCN4a.

### Accelerate Conduction—Increase Membrane Potential

Conduction can be enhanced by increasing membrane potential. This can be achieved by delivery of hMSCs or AAV or other viral constructs containing the ERG3 gene. ERG3 will provide a steady state hyperpolarization. Its advantage is that it will hyperpolarize depolarized tissue but not normally polarized tissue. In other embodiments, an inward rectifier gene, Kir 2.1 or 2.2 may be individually or coexpressed, to increase diastolic K conductance.

In ventricular tachycardia of a partially healed infarct, the viable but depolarized tissue in the border zone (Weerapura, M., et al., (2002), J. Physiol. 540.1, 15-27) provides the substrate for a reentrant arrhythmia. As described above, this requires slow conduction, which is the result of a depolarized membrane potential either due to reduced myocyte K conductance or coupling to less K selective myofibroblasts (Yao, J.-A., et al., (1999), Cardiovasc. Res. 44, 132-145; Kohl, P., et al., (2005), J. Electrocardiol. 38(4 Suppl.), 45-50). One embodiment of the present invention relates to hyperpolarizing the diastolic membrane potential to make more sodium current available. In normal myocytes the diastolic membrane potential is largely set by the inward rectifier IK1 (generated by the genes Kir2.1 with some contribution from Kir2.2) (Nakamura, T. Y., et al., (1998), Am. J. Physiol. 274, H892-H900; Zaritsky, J. J., et al., (2001), J. Physiol. 533.3, 697-710). Although the conductance is large, the membrane potential still sits between 5 and 10 mV positive to the predicted potassium equilibrium potential because of the presence of an inward background current. (Gao, J., et al., (2005), Biophys. J. 89, 1700-1709; Cohen, I. (1983), Experientia 39, 1280-1282). If one can increase diastolic membrane K conductance, membrane hyperpolarization can be achieved making sodium current more available. Thus, one embodiment of the present invention provides expressing the inward rectifier genes in hMSCs to achieve this aim. In another embodiment, a member of the ERG gene family is used. ERG3, which we first identified and expressed (Shi, W., et al., (1997), J. Neurosci. 17, 9423-9432) encodes a delayed rectifier with a steady state “window current” at potentials more positive than −70 mV. Using ERG3 allows hyperpolarization of depolarized tissue while leaving healthy tissue unaffected.

Increasing K channel expression will enhance conduction by increasing resting potential and relieving inactivation of native Na channels, providing a beneficial therapy for VT. As noted above with other method of treatment of the present invention, delivery of the expressed K channel will be implemented from within the myocytes by adenovirus or adeno-associated virus delivery, or other viral delivery and also within coupled hMSCs. Three different channels are preferred to enhance K conductance and R, either an inward rectifier (Kir2.1 or Kir2.2) or ERG3, the latter having the advantage of greater conductance at less negative potentials so that it will preferentially influence already depolarized cells. In the case of ERG3 expression, we can also introduce expression of siRNA or a dominant negative construct to further reduce endogenous I_{K1} current to probe the limits of efficacy of K channel upregulation.

Conduction studies using the MEA system and K channels (Kir2.1, Kir2.2, ERG3) expressed with viral vectors or in hMSCs will demonstrate that K channel over-expression of a K channel restores conduction. In either case, co-cultures of cells expressing and not expressing the channel are prepared with specific patterned growth to study events at transitions zones, and to study the impact of heterogeneous expression of the exogenous channel. In the case of ERG3 expression, studies also will be done when native I_{K1} is suppressed by siRNA or dominant negative expression to cause depolarization and depressed conduction. In other experiments, conduction will be reduced by either membrane depolarization or suppression of the native cardiac Na channel. Na channel suppression will be achieved by expression of siRNA or by use of tetrodotoxin.

Action potential parameters (Vₘₐₓ, Rₘ, etc) will be determined in myocyte cultures or co-cultures of myocytes and hMSCs. In the case of ERG3 expression, studies also will be done when native I_{K1} is suppressed by siRNA or dominant negative expression, and dofetilide will be employed to test for an ERG contribution to the resting potential in this case. Action potential recordings also will be done under conditions of membrane depolarization and Na channel suppression

Accordingly, the present invention provides a method of treating ventricular tachycardia by enhancing conduction by increasing membrane potential of ventricular myocytes. In a preferred embodiment the method comprises delivering to the ventricular myocytes the ERG3 gene. Preferred delivery methods are as discussed previously—by delivering hMSCs or viral vectors expressing said ERG3 gene. In other embodiments, a method of treating ventricular tachycardia comprises delivering to the ventricular myocytes an inward rectifier gene, preferably Kir 2.1 or 2.2 or combination thereof.

### Slow Conduction—SCN5a siRNA

Although it is most desirable to restore normal function, it is also possible to terminate reentrant arrhythmias by blocking sodium channels and converting slow conduction with unidirectional block to bidirectional block. A major reason why current pharmacologic approaches to blocking sodium channels are not optimal is that the blocking agents are not selective (Ritchie, J. M., et al. (1990),Goodman and Gillman's The Pharmacological Basis of Therapeutics” Eighth edition, eds. Goodman Gilman, Rall, Nies & Taylor, Pergamon Press, pp. 311-331). One way to selectively downregulate expression of proteins is to overexpress a protein-specific small interfering RNA (siRNA). The relevant siRNA will hybridize with the mRNA encoding the protein of interest and target the message to RISC complexes in which the mRNA is degraded. SiRNA's for all known proteins are commercially obtainable. Further, as illustrated in preliminary studies, these rod shaped molecules can permeate gap junction channels. Thus, it becomes practical to consider either viral or cell based delivery of siRNA. This suggests the bases of certain embodiments of the present invention—that reentrant arrhythmias could be terminated by delivery of an siRNA against the alpha subunit of the cardiac sodium channel SCN5a. Accordingly, one method of the present invention provides a method of treating atrial fibrillation comprising delivering an siRNA against the alpha subunit of the cardiac sodium channel SCN5a to the atrium or certain desired areas thereof.

### Slow Conduction—Nav1.5 Alpha siRNA

In other embodiments there is provided a method of treating ventricular tachycardia by suppressing conduction completely in a desired location of the ventricle, the method comprising administering to the desired area siRNA against Nav1.5 alpha subunit to induce bidirectional conduction block at the area. See Example 15.

### Slow Conduction—expression of Cx31.9

Regionally reducing gap junctional conductance by overexpressing Cx31.9 will prevent rapid impulse initiation that is focally triggered from propagating beyond its site of origin. While this might seem counterintuitive with regard to the roles generally played by gap junctional proteins, it has been demonstrated that Cx31.9 and its murine counterpart, Cx30.2, operate to decrease gap junctional conductance. The goal is to deliver a connexin that acts as a dominant negative to reduce/slow junctional conductance. If Cx31.9 is acting as a dominant negative, gap junctional coupling between myocyte pairs should be reduced. Focal delivery of Cx31.9 in vivo to specific locales within the heart represents a potential cure for reentrant arrhythmias by converting unidirectional block to bidirectional block. Mouse Cx30.2 the orhthologue of human Cx 31.9 has been shown to be a causative component in the slowing of the action potential in the AV node.

One embodiment of the present invention thus provides a method of treating atrial fibrillation comprising locally reducing gap junction conductance by overexpressing Cx31.9 to prevent rapid impulse initiation that is focally triggered from propagating beyond its site of origin. In certain embodiments, Cx31.9 is delivered via a viral vector capable of expressing Cx31.9. In a preferred embodiment, the viral vector are injected intramyocardially in the base of the left atrial appendage.

Cell coupling will be reduced by adenovirus or adeno-associated virus over-expression of the low conductance gap junctional protein Cx31.9/Cx30.2 within myocytes. These connexins are known to slow conduction in the AV node and may function as a possible dominant negative connexin. The approach has therapeutic relevance in AF, where a localized reduction in conduction can disrupt the reentrant circuit.

### Prolong Effective Refractory Period (ERP)

Atrial fibrillation and ventricular tachycardia can be delayed—a prolonged effective refractory period can be created by slowing deactivation of the delayed rectifier. This can be achieved by (a) delivery of hMSC containing the hERG1 gene without coexpression of MiRP1, or (b) delivery of an siRNA to silence expression of native MiRP1 or (c) delivery of a mutant form of hERG1 which has slowed deactivation kinetics. In so doing ERP is prolonged, but not repolarization.

In another embodiment of the present invention, absolute and relative refractory periods are lengthened such that conduction either fails in both directions or allows the sodium channel to recover from inactivation sufficiently to conduct more normally in both directions. The easiest way to lengthen refractoriness is to increase the action potential duration possibly by blocking delayed rectifier K channels (IKr and IKs). Unfortunately, this would lengthen the action potential, which tends to induce early after depolarizations (EADs) and predispose to the prototypically lethal drug-induced arrhythmia, torsade de pointes (Marban, E. (2002), Nature 415, 213-218). One solution to this problem is to enhance refractoriness by slowing deactivation of K conductance at diastolic potential thereby requiring greater activation of sodium current to generate excitation.

Reentrant arrhythmias require reexcitation of tissue by a propagating waveform. Any circumstance (e.g. slow conduction or more rapid repolarization and recovery of excitability) that facilitates recovery of excitability in the pathway will permit further invasion of that path by the reentering waveform. In the previous section we described approaches to enhance conduction and in that manner reach the point of origin more quickly during the refractory period. An alternative approach is to extend refractoriness so that even under conditions of slow conduction reexcitation is not possible. The easiest way to guarantee refractoriness is to lengthen the duration of the action potential. However, it is well known that prolonged action potential plateaus generate an acquired long Q-T syndrome that predisposes to the lethal ventricular arrhythmia torsades de pointes (Marban, E. (2002), Nature 415, 213-218). Therefore, it would be highly desirable to extend the refractory period without prolonging the action potential. One obvious alternative is to transiently enhance potassium conductance at diastolic potentials. A clue to how this might be achieved comes from the original studies demonstrating a role for the beta subunit KCNE2 (which codes for the protein MiRP1) in the rapid component of the delayed rectifier IKr (Abbott, G.W., et al., (1999), Cell 97, 175-187). When MiRP1 was coexpressed with the alpha subunit hERG in a heterologous expression system, deactivation of the expressed current at hyperpolarized potentials was accelerated. This more rapid deactivation allows for easier excitation with available inward currents. Thus, to extend refractoriness it would be desirable to eliminate the effects of MiRP1 on hERG or alternatively to find mutant hERG channels with even slower deactivation kinetics. In structure function studies using alanine or trytophan scanning mutagenesis (Piper, D. R., et al., (2005), J. Biol. Chem. 280, 7206-7217; Subbiah, R. N., et al., (2005), J. Physiol. 569.2, 367-379) a number of mutant hERG channels had substantially slower deactivation kinetics (see FIG. 8A and FIG. 8b). These mutants K538A and L539W provide extreme examples of slowed deactivation.

In the global approach, either the MiRP1 siRNA or the mutant ERGs is delivered to extend refractoriness. For the focal experiments, first hMSCs are delivered overexpressing hERG without coexpression of MiRP1. Second, hMSCs overexpressing an siRNA against MiRP1 are delivered. In both these cases, a mixture of deactivation kinetics that is slower than the control should be seen. However, one recent report questioned whether MiRP 1 accelerated deactivation kinetics physiologically (Weerapura, M., et al., (2002), J. Physiol. 540.1, 15-27). Therefore as a third focal approach to definitively extend deactivation kinetics the mutant channels hERG K538A or L539W will be expressed their effects on refractoriness determined, either in a myocyte alone or when a myocyte is coupled in a two cell syncytium with a transfected cell.

Accordingly, the present invention provides a method of treating atrial fibrillation or ventricular tachycardia comprises prolonging the refractory period by slowing deactivation of the delayed rectifier in the atrium or ventricle. One method comprises delivering the ERG1 gene or a mutant ERG1 gene having slower deactivation kinetics as compared to ERG1 is delivered to the atrium or ventricle without co-expression of MiRP1. In another embodiment, the mutant ERG1 gene is K538A or L539W. In another embodiment, the ERG1 or mutant ERG1 is delivered via delivering hMSCs transfected with ERG1 or the mutant ERG1 or via delivering a lenti-viral vector capable of expressing ERG1 or the mutant.

In another embodiment, prolonging the refractory period by slowing deactivation of the delayed rectifier in the atrium or ventricle comprises delivering siRNA to the atrium or ventricle to silence native MiRP1 expression.

### Optimizing the Cellular Platform

If cell therapy is to be successful one must optimize the cellular delivery system as well as the genes to be delivered. The optimal cell must meet the following six criteria: 1) allow sustained expression; 2) express connexins to readily integrate into cardiac tissue; 3) do not proliferate; 4) do not differentiate; 5) are not rejected; and 6) are not apoptotic. Previous studies created a biological pacemaker using hMSCs as the cellular delivery vehicle (Potapova, I., et al., (2004), Circ. Res. 94, 952-959). These 3rd passage cells were not rejected after 6 weeks in vivo in the canine heart (Plotnikov, A. N., et al., (2005), Circulation (Suppl.) 112, II-221), and this lack of rejection is consistent with studies from others that suggest this cell type is immunoprivileged (Liechty, K. W., et al., (2000), Nat. Med. 6, 1282-1286). However, these cells proliferate and also have the capacity to differentiate. However, continued passaging of hMSCs reduces their ability to differentiate and slows their rate of cell division. This observation has also recently been reported by others (Bonab, M. M., et al., (2006), BMC Cell Biology 7, 14-20). It has been shown that these late passage cells still make connexins and can be successfully transfected to express exogenous genes.

hMSCs make connexins and can form gap junctions with myocytes. Both hMSCs and cardiac myocytes express Cx43 and when co-cultured form functional gap junctions (Valiunas et al., 2004). FIG. 35 illustrates in vitro data from Valiunas et al., (2004). In addition, hMSCs are also able to intercalate into working myocardium forming gap junction with myocytes and when expressing the pacemaker gene HCN2 can affect pacing in the heart (Potopova et al., 2004). It was concluded that stem cells and myocytes already have an affinity for heterologous gap junction channel mediated coupling. The time course of coupling between hMSCs or model cell lines with myocytes is a critical base line if one wishes to assess changes in the time course of coupling associated with up-regulation of connexin expression. FIG. 36 shows the time course under control conditions for a variety of cell types with isolated canine or rodent ventricular myocytes. The time constant for half maximal junctional conductance under in vitro conditions is 24 hours.

### Delivery of siRNA Via Gap Junction Channels

It has been shown that Polymerase beta expression in wild type cells is significantly reduced when they are co-cultured with cells producing siRNA for Polymerase beta. This occurs only if the cell types express Cx43 (Valiunas et al., 2005). Fluorescence activated cell sorting or FACS was used to separate wild type cells from siRNA producing cells. When connexin deficient cells that normally express Polymerase beta were co-cultured with siRNA producing cells no suppression of Polymerase beta was observed. An extracellular mediated path is inconsistent with these findings as are pinocytotic mechanisms. FIG. 31 illustrates the concept of cellular delivery of siRNA. The cell on the left in FIG. 31 has been transfected with a cDNA for a hairpin siRNA (shRNA). The action of Dicer RNAse (common to all cells) produces an dimerized siRNA which is in equilibrium with single stranded forms. The right-hand cell represents a wild type cell receiving siRNA via gap junction.

HMSCs express connexins, can form functional gap junctions, can heterologously express ion channels, and pass oligonucleotides. Human mesenchymal stem cells (hMSCs) represent an autologous cell population that forms gap junctions composed of connexin43 and connexin40 (Valiunas et al., 2004). Genetic engineering of the hMSCs along with the expression of connexins and their consequent gap junctional coupling to cardiac myocytes make hMSCs an ideal cellular delivery system (Potapova et al., 2004). Further, because hMSCs form gap junctions composed of connexin43, siRNA molecules can be delivered to any cell with which an hMSC can form connexin43 based gap junctions (Valiunas et al., 2005).

Endogenous and exogenous small interfering RNAs (siRNAs) between 20-24 nucleotides in length, profoundly affect gene expression (Elbashir et al, 2001; Caplen et al., 2003; Xu et al., 2004; Miller and Grollman, 2003). The ability of siRNA to affect the synthesis of specific proteins illustrates the importance of this class of molecule in regulating cellular function. A growing understanding of the role of siRNA has made it a potential therapeutic tool (Hampton, 2004). Although the action of siRNAs is highly specific, the ability to target exogenous siRNAs to particular locations and deliver them intact to the interior of target cells in vivo has been problematic (Vomlocker, 2005). For this reason, the possibility of the cell-based siRNA delivery system illustrated in FIG. 31 was considered.

SiRNA stability is an important factor in the cell to cell transfer of siRNA via gap junctions. Endogenous and exogenous siRNAs survive and remain functional for hours or days (Alisky and Davidson, 2004). Given their prolonged survival and the fact that siRNAs permeate gap junction channels implies they may influence not only the cell in which they are produced, but also adjacent and perhaps even distant cells of a syncytium. Thus a small group of cells could potentially use this mechanism to alter organ function. Clearly, the ability to deliver siRNA to the interior of a cell in a target tissue exclusive of the extracellular space has significant therapeutic potential for a number of disease states including arrhythmias.

### Mammalian Gap Junction Channel Permeability

Early studies on gap junction channels composed of connexins generated a consensus view that they were not permeable to molecules with molecular weights greater than 1.5 kD (Simpson et al., 1977; Schwartzmann et al., 1981) or with minor diameters greater than 1.2-1.3 nm (Neijssen et al., 2005). Recent illustrations showing the passage of rod-shaped oligonucleotides and siRNA with minor diameters of 1.2 nm or less but with major diameters of 3-8 nm (Valiunas et al., 2005) and weights up to 4-5 kD has redefined the limits of gap junction channel permeability. More importantly a demonstration of the ability of siRNA to traverse gap junction channels (Valiunas et al. 2005) adds yet another dimension to the role gap junction channels play in coordinated tissue functions. Besides allowing the movement of monovalent ions and second messengers and metabolites, gap junctions are also able to mediate gene silencing.

One must keep in mind that not all connexins behave the same. The single channel conductance and selectivity/permselectivity of gap junction channels is highly dependent on the type of subunit connexin (Goldberg et al., 2004; Valiunas et al., 2002; Ek-Vitron et al., 2006). For example Cx43 single channel conductance measured in Cs or K salt is ±90 pS while that of Cx40 is 140 pS. The selectivity/permselectivity properties also differ. The permeability ratio for Lucifer Yellow relative to K+ is 1/40 for Cx43, and 1/400 for Cx40 (Valiunas et al., 2002). In a recent study Ek-Virton et al., (2006) found that LY permeability to Cx43 was less than that for cations. It appears to be very similar to that reported by Valiunas et al. (2002). It was also shown that the phosphorylation state can affect the permeability of cationic species dramatically. In fact, small cationic probes can attain permeabilities similar to K+ when Cx43 is phosphorylated. A number of studies have compared the relative transfer rate of endogenous solutes such as nucleotides and second messengers for a number of connexins. As was the case for the exogenous probe Lucifer Yellow, endogenous probe permeability is highly dependent on the connexin type. A number of publications have illustrated that the various connexins have different permeabilities to a variety of probes; Goldberg et al., 2004; Niessen et al., 2000). Other connexin types that have not been studied as completely as Cx43 or Cx40 are Cx37 found most abundantly in endothelium (Beyer 1993) and Cx45 found in select regions of the heart and vascular wall. Table 3 lists the unitary conductances and permeability ratios for Lucifer Yellow (LY), to K+ where known, for the connexins to be tested for oligonucleotide permeability and gene silencing capability.

TABLE 3

| | homotypic unitary | |
|---|---|---|
| Connexin type | conductance (pS) | *LY/K+ |
| Cx37 | 400 (Veenstra et al., 1994) | — |
| Cx46 | 140 (Trexler et al., 2000) | — |
| Cx31.9 | 15 (Srinivas et al, 2002; White | — |
| | etal. 2002) | |
| Cx32 | 60 (Oh et al., 1999) | — |
| Cx40 | 140 (Valiunas et al., 2002) | 1/400 |
| Cx43 | 90 (Valiunas et al., 2002) | 1/40 |
| Cx45 | 27 (Goldberg et al., 2004) | 1/100 |

*ratio of Lucifer Yellow to K+ flux per channel (Valiunas et al., 2002).

The list demonstrates the diversity of conductances ranging from 400 pS for Cx37 to 27 pS for Cx45. Interestingly while Cx45 has a smaller conductance than Cx43 or Cx40, it is more permeable to Lucifer Yellow than Cx40. These data point to the diversity of properties in the multigene family of connexins. The varied permeabilities and conductances of homeotypic gap junction channels are also apparent with regard to the cell to cell transfer of oligonucleotides and siRNA. The data published by Valiunas et al., (2005) show that Cx43 allows the passage of siRNA while Cx32/Cx26 heteromeric channels do not. Preliminary data using morpholinos also indicate that homeotypic Cx32 and Cx26 gap junction channels fail to pass oligonucleotides (see Valiunas et al., 2005 in the appendix). Permeability characteristics under conditions of pharmacologically-stimulated up-regulation or over-expression of catenins and N-cadherins in hMSCs will be assessed to determine the optimal connexin to employ for siRNA transfer.

Overexpression of exogenous connexins can enhance the efficacy of siRNA transfer from delivery cell to target cell. Cx46 manifests as a large single channel conductance and might well allow the permeation of siRNAs.

FIG. 38 is a summary graph of oligonucleotide permeability for nucleotides of different lengths, TEA and Lucifer Yellow relative to K ion for Cx43. Cell types expressing Cx43 include hMSCs. The data are taken from Valiunas et al., 2002; 2005; Goldberg et al., 2004; Weingart, 1974). FIG. 39 shows transfer of 12 mer between a HeLa cell pair expressing Cx40 (Valiunas et al., 2002). These data show that Cx40 is also able to pass oligonucleotides. Cx40 will be probed with larger morpholinos. Data is to be collected to allow for a quantitative analysis and comparison with Cx43.

siRNA can permeate gap junction channels and silence genes in wild type cells. The silencing of Polymerase Beta in wild type NRK cells via the transfer of siRNA targeted for Polymerase beta from NRK cell transfected with siRNA was previously reported (Valiunas et al., 2005). Knockdown of Polymerase beta occurred in cell types expressing Cx43, NRK cells are known to express Cx43 (Musil and Goodenough, 1991). FIG. 40 shows the summary histogram taken from a recent publication (Valiunas et al., 2005). Only when cells are coupled by Cx43 can siRNA producing cells effect a reduction in Polymerase beta. In cases where communication incompetent cells (parental N2A cells) are used no knockdown is observed. Transfer of siRNA occurs when cells are coupled by Cx43 but not in any other condition tested. These data eliminate an extracellularly mediated path.

The present invention also provides compositions useful in the manufacture of a medicament to treat atrial fibrillation or ventricular tachycardia. Various constructs mentioned above in the described methods would be useful in compositions for treating the AF or VT.

### EXAMPLES

### Example 1

### Gene and Cell Therapy can be Achieved—the Creation of a Biological Pacemaker

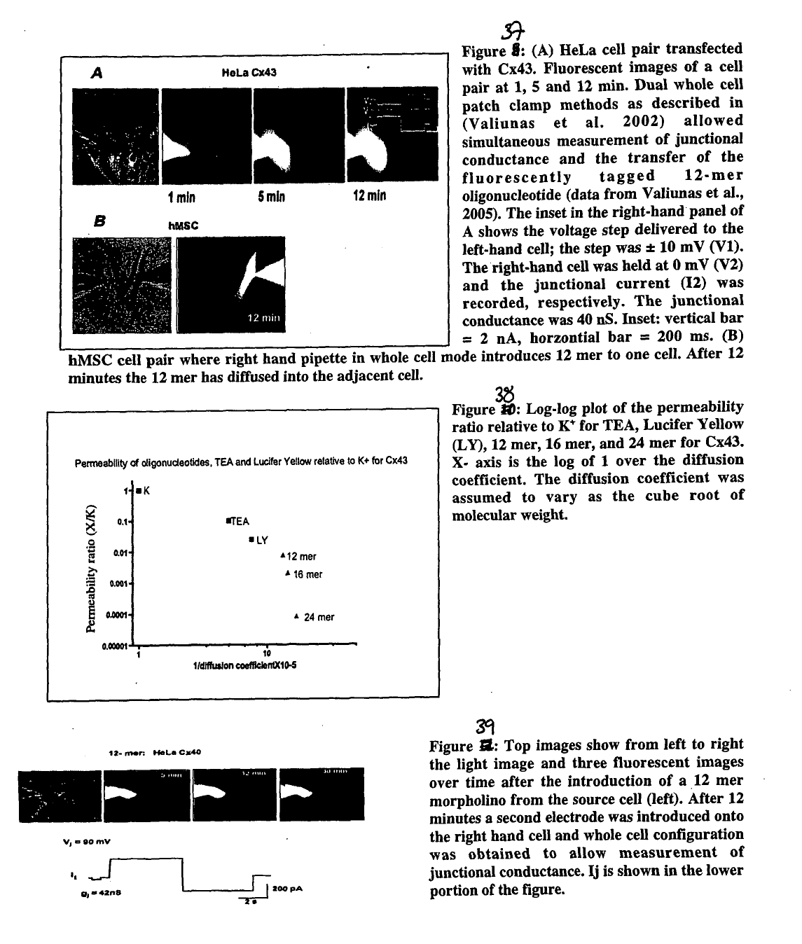

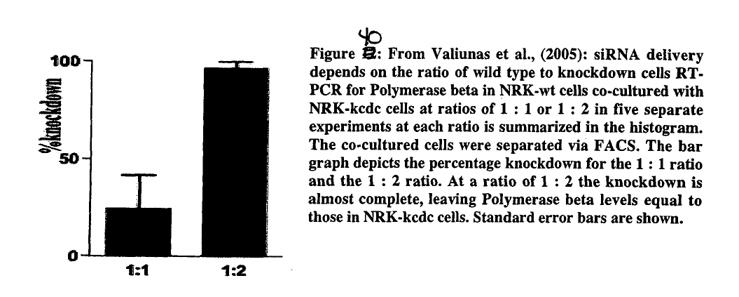

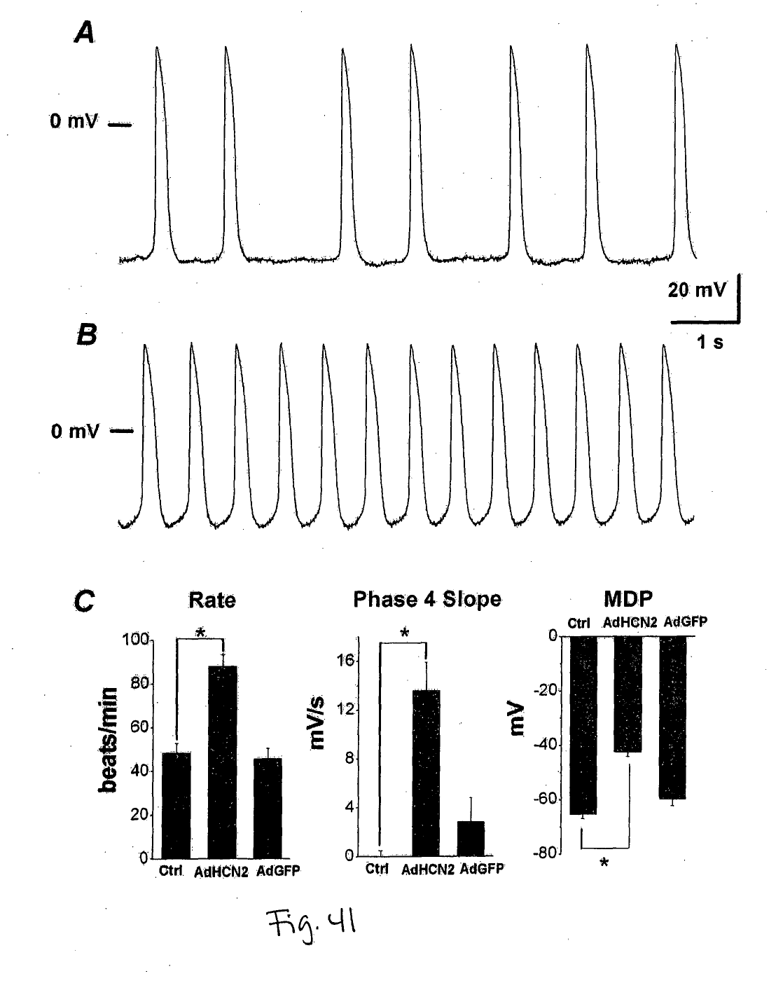

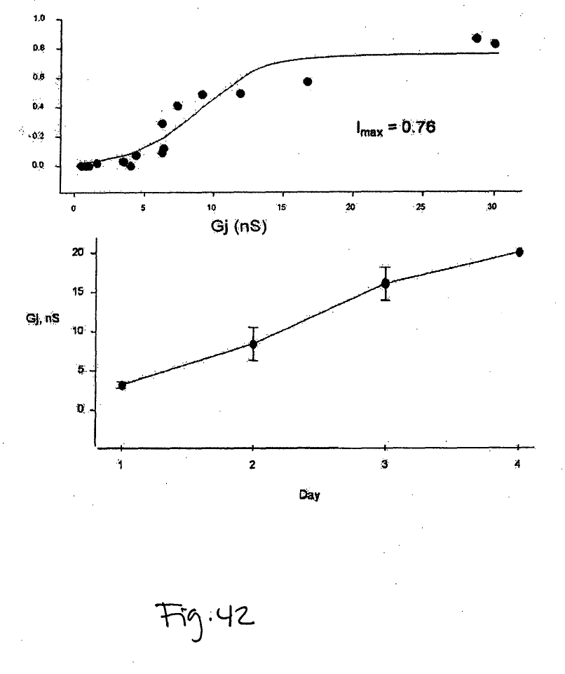

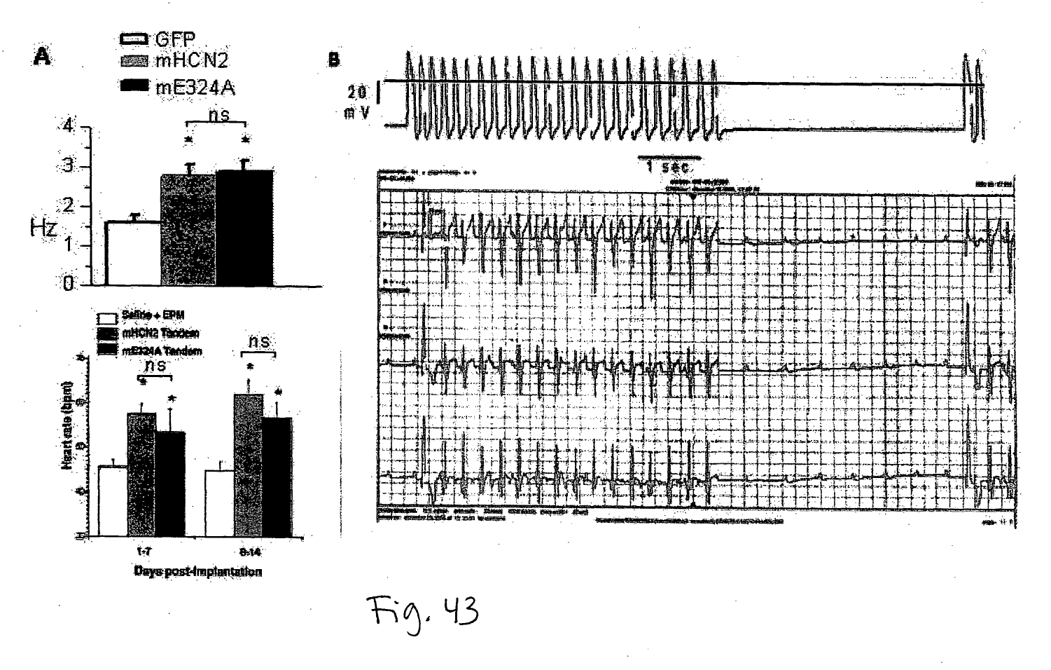

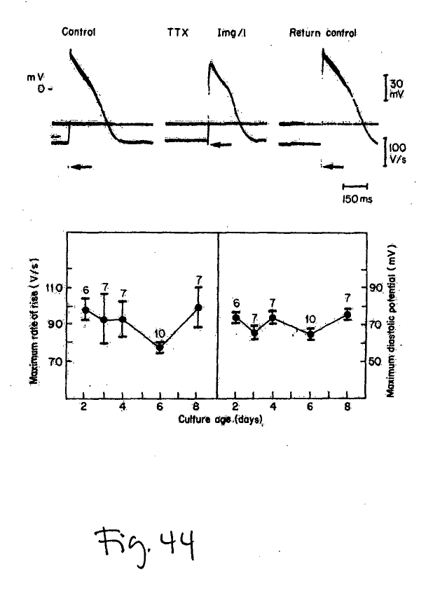

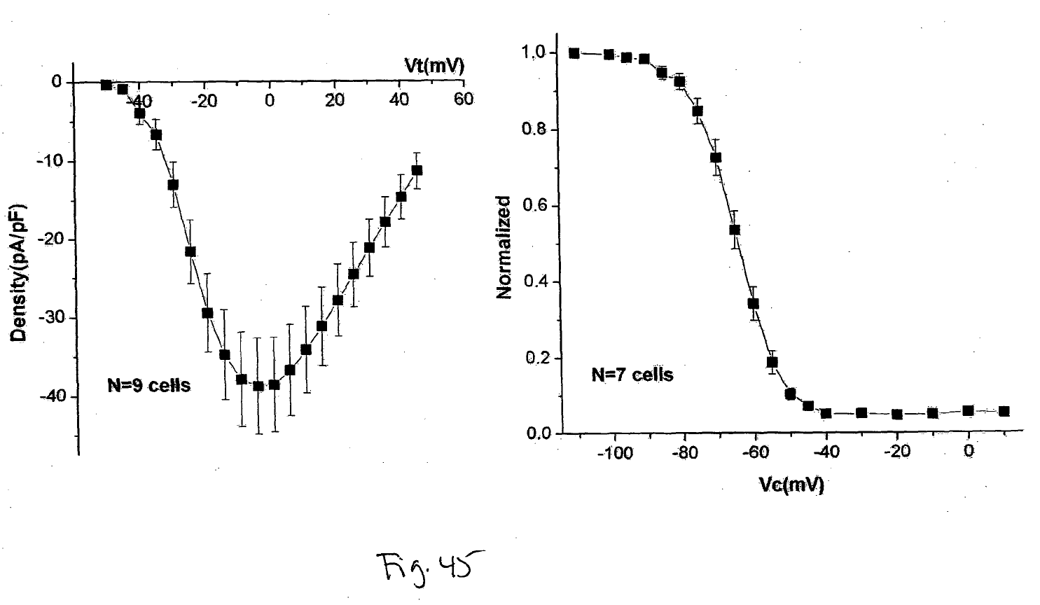

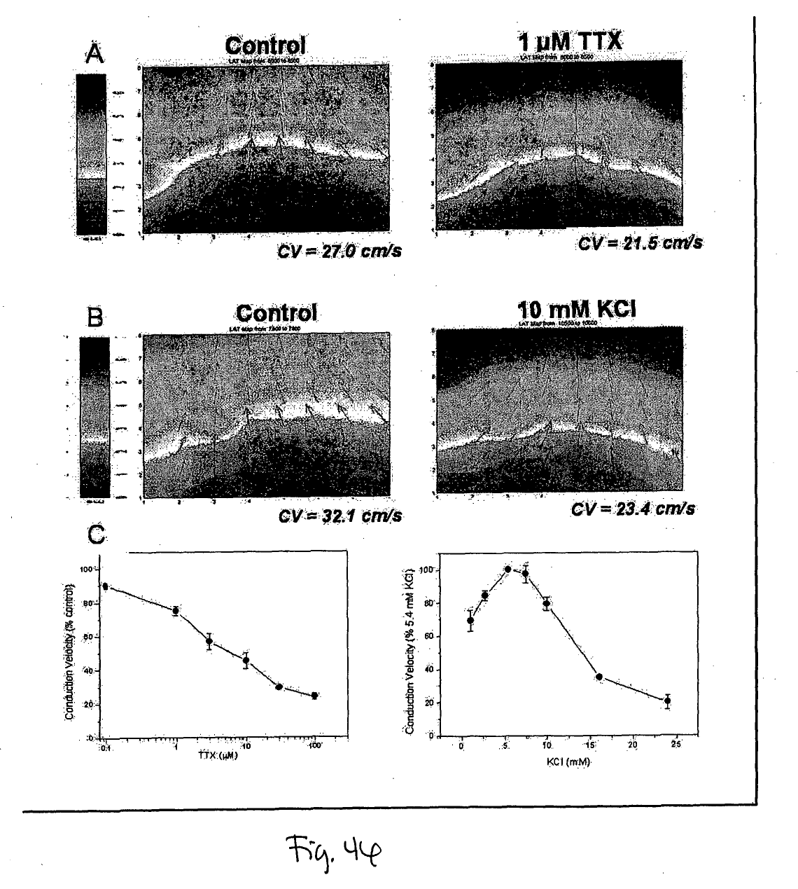

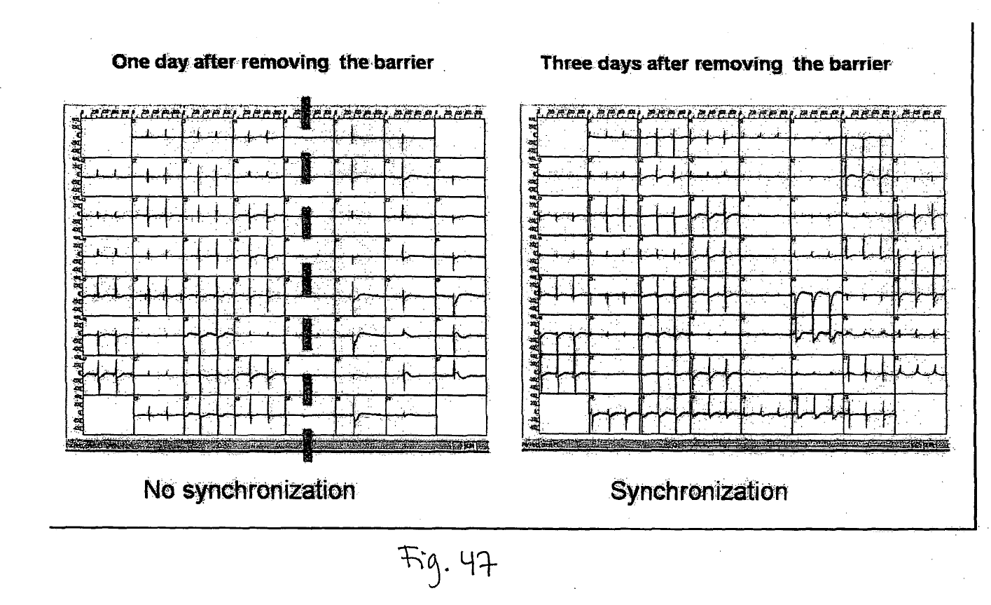

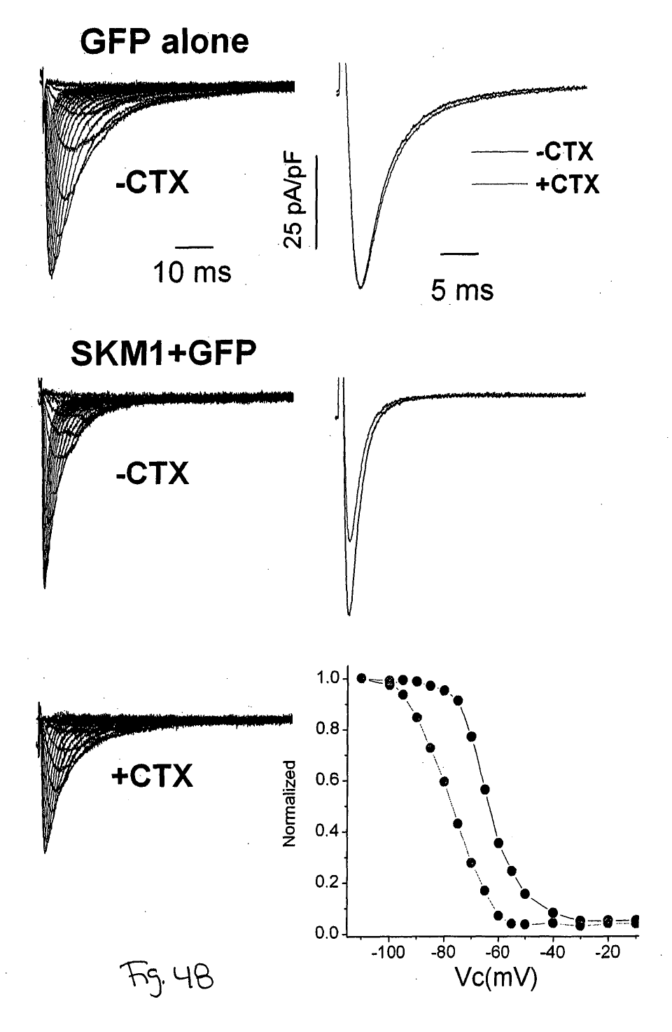

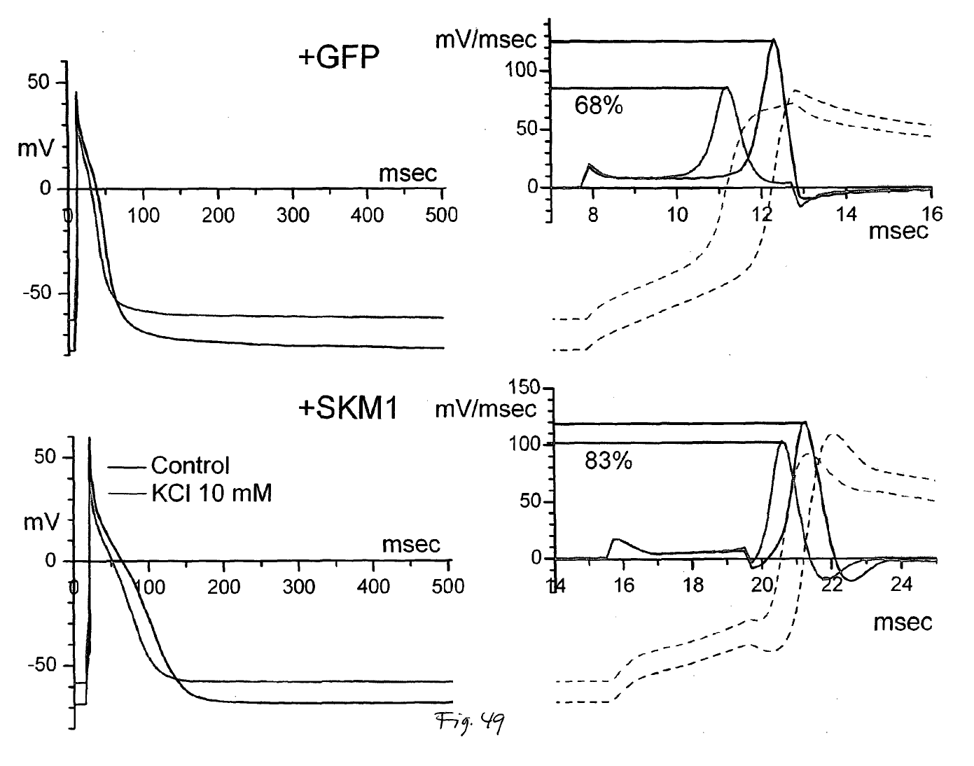

The use of the mHCN2 construct delivered in an adenovirus globally to the left atrium of the canine heart to create the first biological pacemaker based on a family member of the molecular correlate of the native “pacemaker current” I_{f} has been reported. The same construct was tested in a canine ventricular conducting system. In both cases biologic pacemaker activity was evident and in the case of the conducting system delivery, it was sufficient to generate physiologic rates. However adenoviral delivery is transient and within two weeks the biological pacemaking had disappeared. In order to gain greater persistence, a cellular platform for delivering the gene was tested. Previous work on human mesenchymal stem cells (hMSCs) had shown that they could be transfected by electroporation (Hamm, A., et al., (2002), Tissue Eng. 8, 235-245.) so viruses were not necessary. One additional advantage of this cell type was the suggestion from previous work that they were immunoprivileged (Liechty, K. W., et al., (2000), Nat. Med. 6, 1282-1286). In order for a cell type to serve as a platform it had to produce connexins (or be transfected with them) and so initial studies demonstrated their ability to couple with cells expressing the cardiac connexins 40, 43, or 45 or to adult canine ventricular myocytes. (Valiunas, V., et al., (2004), J. Physiol. 555, 617-626). Next it was necessary to demonstrate that the hMSCs could be transfected with the HCN2 gene by electroporation and express HCN2 induced current (See FIG. 9). If this biological pacemaker was to be functional, it must also be regulated by the autonomic nervous system. The HCN2 protein has a cyclic AMP binding site and is known to be regulated by this second messenger (Hamm, A., et al., (2002), Tissue Eng. 8, 235-245). FIG. 10 shows that the beta agonist isoproterenol has a direct effect on the expressed current while FIG. 11 demonstrates that acetylcholine (Ach) only has an effect in the presence of the beta agonist (accentuated antagonism). Once the hMSCs were confirmed to exhibit the desired biologic activity they were then tested in a model system. HMSCs expressing either GFP alone or GFP+HCN2 were plated onto a cover slip within a cloning cylinder to form a “node” and then neonatal myocytes were plated over the node and 4 days was allowed for effective coupling of the stem cells to the myocytes. The coculture containing stem cells expressing the HCN2 gene had a much higher spontaneous rate (164 bpm vs. 93 bpm <0.05). Finally hMSCs were tested in vivo in the canine heart. Again either GFP or GFP+HCN2 hMSCs were employed and roughly 1 million stem cells were injected into the left ventricular free wall. Three to ten days were allowed before detailed studies were performed. All four control animals (hMSCs expressing GFP) had spontaneous rhythms (2 in each ventricle) with an average rate of 45 bpm. In the test group (hMSCs expressing HCN2+GFP) 5 of the 6 animals had left sided rhythms with an average rate of 61 bpm (P<0.05). The sites of the injection were excised and studied by immunocytochemistry. The tissue showed evidence of basophilic cells that stained positive for both human cd44 and vimentin. Connexin staining demonstrated that these cells made gap junctions with each other and with cardiac myocytes. These studies demonstrate the feasibility of delivering exogenous genes to model systems or the in vivo canine heart to treat an arrhythmmogenic substrate. Embodiments of the present invention extend the work from delivery of HCN genes to delivery of Na and K channel genes or relevant siRNA against their alpha or beta subunits.

### A. The Time Course and Effectiveness of Gap Junctional Coupling

If cell therapy is to be effective it is necessary that the genetically engineered cell couple to the native myocytes and that this coupling effectively transfer the electrical signal carried by the delivered cell. FIG. 12 shows the time course of coupling in vitro between stem cells and either neonatal rat (filled circles) or adult canine (open circles) ventricular myocytes. Full coupling takes 72 to 96 hours but 10 nS of gap junction conductance occurs within 48 hours of coculture. Similar results were also obtained examining coupling between HeLA cells and canine myocytes (red triangles). The effectiveness of a given level of cell to cell coupling was tested using N2A cells that were transfected with Cx43. One cell of the pair was also transfected with the HCN2 gene. FIG. 13 illustrates the protocol used to assess the effectiveness of coupling as a function of the gap junctional conductance. One cell of the pair was voltage clamped and the amplitude of the HCN2 induced current recorded while the other cell of the pair was in current clamp mode. Then the other cell of the pair was voltage clamped with an identical protocol. FIG. 13 shows there was 30 nS of gap junctional coupling and most of the HCN2 induced current could be recorded by voltage clamping the cell that was not transfected. FIG. 14 relates the magnitude of gap junctional conductance to the effectiveness of transfer of HCN2 induced current. Roughly half of the current is observed at a 10 nS coupling conductance which occurs within about 48 hours of coculture.

B. Measuring Sodium Currents and Results with the SkmI Sodium Channel

Studies of the sodium channel began in 1979 by reporting the existence of a steady state component of current called the “TTX-sensitive window current” (Attwell, D., et al. (1979), Pflugers Archiv. 379, 137 142). This was the current generated in the steady state that flowed through sodium channels due to the non-zero product of m and h. Slow inactivation of sodium current was reported (Gintant, G., et al. (1984), Biophys. J. 45, 509 512). This inactivation took many seconds and is the basis of what was later renamed persistent sodium current (Saint, D. A., et al., (1992), J. Physiol. 453:219-231). It is this persistent sodium current that has been shown to be altered in one form of long Q-T syndrome in which the fraction of slowly inactivating current is enhanced (Bennett, P. B., et al., (1995), Nature 376, 683-685). For the current application it was necessary to find a sodium channel gene which had a more positive inactivation versus voltage relationship than found normally in cardiac myocytes. The skeletal muscle sodium channel was investigated because of its reported midpoint of inactivation of −68 mV (Hayward, L. J., et al., (1996), J. Gen. Physiol. 107, 559-576). FIG. 15 shows the expression of this current in hMSCs where its midpoint of inactivation is −62 mV.

### C. ERG Currents and Relevant Preliminary Results

Previously, there was uncertainty about the universality of the current IKr. Some action potentials like that of the rat showed little plateau and initial studies did not suggest that IKr was present. In a paper in Circ. Res (Wymore, R. S., et al., (1997), Circ. Res. 80, 261-268), it was demonstrated that the message for ERG1 was present and it was also demonstrated by an appropriate patch clamp protocol that IKr was present in rat ventricular myocytes. Whether other ERG channels might exist was investigated. Two new family members ERG2 and ERG3 were discovered and characterized by their distribution and electrophysiology (along with ERG1) by heterologous expression in Xenopus oocytes. One major difference between the different ERG family members is the magnitude of their steady state “window” conductance. This difference is illustrated in FIG. 16. ERG3 has the largest steady state conductance. This conductance is maximal at −50 mV and is small at −80 mV. hERG1 is used in investigating slowed deactivation as an antiarrhythmic therapy. ERG3 is used in investigating hyperpolarization of the diastolic membrane potential as an antiarrhythmic therapy for depolarized tissue.

D. Inward Rectifiers and Relevant Results with Kir2.1 and Kir2.2

Inwardly rectifying K currents have previously been studied. It was reported that thallium was more permeant than K through IK1 (Cohen, I. et al., (1986), J. Physiol. 370, 285-298) using the two electrode voltage clamp technique and canine Purkinje fibers. It was also shown that the time dependent activation of IK1 in ventricular myocytes was much slower when the internal [K] was reduced (Cohen, I. S., DiFrancesco, D., Mulrine, N. K. and Pennefather, P. (1989). Internal and external K⁺ affects the gating of the inward rectifier in cardiac Purkinje myocytes. Biophys. J. 55, 197-202). Further, it was shown that there was another voltage dependent process that regulated activation of IK1 in the absence of intracellular Mg. A model was created to represent this residual voltage dependent gating (Oliva, C., Cohen, I. S. and Pennefather, P. (1990). The mechanism of rectification of i_{K1} in canine Purkinje myocytes. J. Gen. Physiol. 96, 299-318) that was later determined to be block by polyamines (Lopatin, A. N., Makhina, E. N. and Nichols, C. G. (1994). Potassium channel block by cytoplasmic polyamines as the mechanism of intrinsic rectification. Nature 372, 366-369). In the present invention, inward rectifiers Kir2.1 and Kir2.2 have been expressed in human mesenchymal stem cells (see FIGS. 17A and 17B) and in cell lines.

### E. Ion Channel Beta Subunits

Ion channel beta subunits, such as Mink, have been expressed in oocytes (Cui, J., Kline, R. P., Pennefather, P. and Cohen, I. S. (1994). Gating of I_{sK} expressed in Xenopus oocytes depends on the amount of mRNA injected. J. Gen. Physiol. 104, 87-105). The study analyzed the kinetics of activation of the delayed rectifier current it elicited and suggested that there were multiple open states for the channel. Its partnership with KCNQ alpha subunits to create the slow delayed rectifier iKs was only discovered later (Sanguinetti, M. C., Curran, M. E., Zou, A., Shen, J., Spector, P. S., Atkinson, D. L. and Keating, M. T. (1996). Coassembly of K_{V}LQT1 and minK (IsK) proteins to form cardiac I_{Ks} potassium channel. Nature 384, 80-83; Barhanin, J., Lesage, F., Guillemare, E., Fink, M., Lazdunski, M. and Romey, G. (1996). K_{V}LQT1 and IsK (minK) proteins associate to form the I_{Ks} cardiac potassium current. Nature 384, 78-80). More recently the association of MiRP1 with the HCN gene family has been shown (Yu, H., Wu, J., Potapova, I., Wymore, R.T., Holmes, B., Zuckerman, J., Pan, Z., Wang, H., Shi, W., Robinson, R. B., El-Maghrabi, M. R., Benjamin, W., Dixon, J., McKinnon, D., Cohen, I. S. and Wymore, R. (2001). MinK-related peptide 1. A Subunit for the HCN ion channel subunit family enhances expression and speeds activation. Circ. Res. 88, e84-e87). The results demonstrated that MiRP1's action to increase the amplitude of heterologously expressed HCN1 and HCN2 currents along with speeding its activation kinetics were not shared by another family member MinK. We also demonstrated that MiRP1 is more highly expressed in SA node than in ventricle. This suggests a potential role of MiRP1 in regulating two important currents involved in pacemaker activity (Ikr and If). It was also demonstrated that when HCN1 and MiRP1 are coexpressed in oocytes they coimmunoprecipitate with each other. More recently, it was demonstrated that the biophysical changes we observed in heterologously expressed HCN currents in oocytes are reproduced when MiRP1 is overexpressed in myocytes (Qu, J., Kryukova, Y., Potapova, I. A., Doronin, S. V., Larsen, M., Krishnamurthy, G., Cohen, I. S. and Robinson, R. B. (2004). MiRP1 modulates HCN2 channel expression and gating in cardiac myocytes. J. Biol. Chem. 279, 43497-43502).

F. Small Interfering RNA (siRNA) can be Delivered Via Gap Junctions:

In a specific embodiment of the invention, ion channel subunits may be down-regulated. SiRNA typically about 22 bases long is commercially obtainable for any gene. Global delivery to a specific heart chamber can be achieved by viral approaches, but cell based therapy would require that siRNA permeate gap junction channels. SiRNA is a rod shaped molecule of roughly 10 nm minor diameter. This is similar to the diameter of gap junction channels. The ability of siRNA to transfer through gap junction channels and silence gene expression in coupled wild type cells (Valiunas, V., et al., (2005), J. Physiol. 568.2, 459-468). Morpholinos of 12, 16 and 24 bases in length with a fluorescent tag were synthesized. FIG. 18 shows the transfer over time of a morpholino of length 12 bases between two cells expressing Cx43. There is measurable transfer in a period of only a few minutes. This transfer is slower for longer molecules. Even for the longest morpholinos tested, of 24 bases in length (which is longer than siRNA), there is measurable transfer in a 40 minute period. Not all connexins formed gap junctions that allowed morpholinos to permeate. Gap junctions formed by Cx43 did, but those formed by a combination of Cx32/26 did not. More recently, it has been shown that Cx40 gap junctions pass morpholinos although more slowly than those formed by Cx43. Morpholinos were used as a model system for siRNA. These molecules are not broken down. However, siRNA has a limited lifetime (Alisky, J. M. and Davidson, B. L. (2004). Towards therapy using RNA interference. Am. J. Pharmacogenomics 4, 45-51) and thus it seemed possible that no measurable physiologic effect might be obtainable by gap junction transfer even if permeation was possible. Thus, the ability of cells containing an siRNA against a DNA repair enzyme, polymerase beta, to transfer their siRNA to wild type cells and reduce wild type message levels was tested. Expression was tested with three cell types, those expressing Cx43, those expressing Cx32/26 and cells expressing no connexins (to rule out an extracellular path). The mRNA was reduced only in wild type cells if they expressed Cx43.

G. Ion Channels can be Down Regulated by siRNA and this Reduction can be Measured by the Patch Clamp Technique:

Since the approach to downregulate native ion channel subunits is dependent on siRNA, the ability to use siRNA to downregulate the expression of an ion channel was tested. HCN2 was chosen because of the experience with heterologous expression of this gene. FIG. 19 shows sample data from two cells, one in which HCN2 was expressed and 96 hours later the cell was patch clamped, and a second cell which was first transfected with HCN2 and 48 hours later siRNA was delivered by lipofectin. This cell was also studied 96 hours after the original transfection with HCN2. To be certain the effect was specific to HCN2, 3 sense siRNA's and one nonsense siRNA was tested to rule out non-specific effects of our siRNA transfection procedure. The results are also shown in FIG. 19. There is a significantly larger effect of sense siRNA than nonsense siRNA. This is the approach to be employed in defining an siRNA against the cardiac sodium channel alpha subunit and the beta subunit for hERG, MiRP1. Appropriate siRNA will be inserted into an appropriate plasmid to allow later use for both viral and cell based therapies.

### H. Adenoviral Infection of Adult Cardiac Myocytes In Vitro

To investigate the effect of cellular background on the biophysical characteristics of the expressed current, neonatal and adult rat ventricular myocytes were infected with an adenovirus carrying the pacemaker gene HCN2 (Qu, J., Barbuti, A., Protas, L., Santoro, B., Cohen, I. S., and Robinson, R. B. (2001). HCN2 overexpression in newborn and adult ventricular myocytes. Distinct effects on gating and excitability. Circ. Res. 89, e8-e14). The results are illustrated in FIG. 20. The voltage dependence of activation was dramatically affected by the cell background being about 20 mV more positive in the neonatal myocytes. The basis of this difference may relate to heretofore undiscovered beta subunits or to posttranslational modifications of the alpha subunit. One possible difference might be that levels of src kinases may differ in the two cell types (Arinsburg, S. S., Cohen, I. S. and Yu, H.-G. (2006). Constitutively active Src tyrosine kinase changes gating of HCN4 channels through direct binding to the channel proteins. J. Cardiovasc. Pharmacol. 47, 578-586).

I. Late Passage hMSCs—an Optimal Cellular Delivery System

An optimal cellular delivery system would have six properties: 1) allow sustained transgene expression; 2) express connexins; 3) does not proliferate; 4) does not differentiate, 5) is not rejected; and 6) is not apoptotic. A comparison of early (3-5) and late (>7) passage hMSCs has been performed. FIG. 21A and FIG. 21B shows that both can be effectively transfected with a transgene by electroporation. FIG. 22 shows that both express abundant levels of Cx43. FIGS. 23A and B shows that although early passage hMSCs can be effectively induced to differentiate along an adipogenic lineage such is not the case for late passage hMSCs. FIG. 24 shows that DNA laddering characteristic of apoptotic cells is absent from both early and late passage hMSCs. FIG. 25 shows that caspase activation is also not higher in later passages hMSCs. FIG. 26A shows that late passage cells proliferate less readily than those from earlier passages although cell division is still measurable. FIG. 26B shows that BrdU incorporation is markedly reduced in later passage hMSCs. Reports suggests that proliferation is virtually absent from hMSCs that have been passaged at least 30 times (Bonab, M. M., Alimoghaddam, K., Talebian, F., Ghaffari, S. H., Ghavamzadeh, A. and Nikbin, B. (2006).

J. The Location of the Delivered hMSCs can be Tracked and their 3-D Distribution Reconstructed

If stem cell therapy for cardiac arrhythmias is to be effective, it will be necessary not only to deliver genetically engineered cells but to track their location in vivo. Although initial studies using GFP allowed localization of the cells we delivered, it is difficult to reconstruct the location of all the cells using this fluorescent protein due to the high autofluorescence of the cardiac milieu. Thus, a novel tracking technique using quantum dot nanoparticles (QDs) has been developed (Rosen A B, Kelly D J, Brink P R, Schuldt M T, Lu J, Potapova I A, Doronin S V, Robinson R B, Rosen M R, Gaudette G R, Cohen I S. (2006) Finding Fluorescent Needles in the Cardiac Haystack Reconstructing the three dimensional distribution of quantum dot-loaded human mesenchymal stem cells injected into the rat ventricle in vivo. 3^{rd} Annual Symposium of the American Heart Association Council on Basic Cardiovascular Sciences—Translation of Basic Insights Into Clinical Practice. Keystone, C O; Rosen, A. B., Kelly, D. J., Schuldt, A. J. T., Lu, J., Potapova, I. A., Doronin, S. V., Robichaud, K. J., Robinson, R. B., Rosen, M. R., Brink, P. R., Gaudette, G. R. and Cohen, I. S. (2006). Finding fluorescent needles in the cardiac haystack: tracking human mesenchymal stem cells labeled with quantum dots for quantitative in vivo 3-D fluorescence analysis. (submitted). These highly fluorescent nanoparticles were thought to have potential for cell tracking but their use was limited by the absence of a method for uniform loading. FIG. 27 illustrates that the previously used techniques of electroporation or lipid mediated transfection result in non-uniform loading of QDs while hMSCs loaded with quantum dots by a novel passive loading technique developed in our laboratory are uniformly labeled. Fluorescence activated cell sorting was used to demonstrate that more than 96% of the hMSCs are loaded in this manner. Finally, the hMSCs were delivered to the rat heart and the animals were sacrificed after 1 hour or 1 day after injection. The hearts were then sectioned and studied. FIG. 28 shows low magnification images for the 1 hour animal at the plane of the stem cell injection illustrating the ease with which the fluorescence of the red quantum dots can be observed in the needle track above background autofloresence. The inset illustrates the sustained uniform loading of the QDs in the delivered hMSCs. FIG. 29A provides an image from a 1 day animal. Again even at low power the QD flouresence is easily observed above autofloresence. Over 100 sections were studied and custom algorithms were written to reconstruct the locations of all QD labeled cells FIG. 29B. FIG. 29C provides this three dimensional picture. Finally also illustrated in FIG. 29D is the distance of each of the stem cells from the centroid of the stem cell mass. More than 95% of the hMSCs were within 1.5 mm of this position. For these conclusions to be valid it was necessary to demonstrate that false positives did not occur. This might happen via (1) transfer of the QDs through gap junctions or (2) lysis of the delivered cells and uptake of the naked dots from the lysis product into the myocytes. Control experiments ruled out both of these alternatives.

### Example 2

### Animal Models

### Atrial Fibrillation

There are a variety of AF models, ranging from spontaneous, through atrial pacing-induced tachycardia models having variable degrees of ventricular failure (with rapid ventricular pacing increasing the extent of the failure), through valvular-insufficiency-induced (obtained by inducing mitral or tricuspid regurgitation) and atriotomy-induced (by lesioning the right atrial free wall and then pacing). Research has been performed on dogs, cats, goats and other animal models. More recently, AF has been induced in transgenic mice, as well.

Each model has specific advantages with regard to species, temporal evolution of AF and reproducibility of AF. Tachy-pacing-induced AF was generally popularized by Allessie et al in experiments in the goat (paralleled by Morillo's experiments in the dog). This work generated the hypothesis that atrial fibrillation begets atrial fibrillation, by showing that recurrences of the arrhythmia facilitated its further (and more sustained) occurrence. This model also has shown properties of advancing from paroxysmal to persistent to permanent status that has been used to characterize human AF as well. A modification of the model described by the Nattel group has incorporated rapid ventricular pacing for 1-2 weeks in the early evolution of the AF. This has the advantage of speeding the evolution of AF, but also modifies its drug response. Specifically, AF induced by rapid pacing alone responds well to flecainide and similar drugs—much like AF in humans, but far less well to dofetilide. In contrast, AF associated with congestive failure in the canine model responds to both drugs. This pattern of drug response reflects the human clinical condition quite accurately. It is important to emphasize that chronic, rapid atrial pacing does give rise to congestive failure (Donahue, CV Res). Hence we can achieve our end-point of congestive failure without the necessity of surgically creating valvular insufficiency and/or ventricular outflow obstruction.

Also of importance is the site of pacing with regard to AF initiation. In efforts to mimic the impulse initiation that arises when triggered foci occur in the coronary sinus and pulmonary veins we have used selective right and left atrial pacing. There is definite regional variability in the ability to initiate AF and the rate of its evolution, with left sided sites being far more effective here than right sided.

Regardless of whether initiating beats are sinus, triggered or automatic from ectopic atrial foci it is clear that AF is a reentrant arrhythmia. As such, it is susceptible to interventions that upset the balance among path length, conduction velocity and refractoriness that characterizes AF. A key in facilitating studies, is a chronic canine model that maintains a constant path length while permitting evaluation of the effects of interventions on conduction and refractoriness in a relatively short period of time. The model is adapted from that described by Rosenblueth and Garcia Ramos, modified by Hoffman et al. and then again by Ishii et al. This surgical scar model has the advantage of being readily reproducible and becoming arrhythmogenic and producing atrial flutter and AF within a few days. Its reproducibility over the short-term confers a major advantage for proof-of-concept experiments and also permits the use of short-lived vectors such as adenovirus. Moreover, the surgically scarred right atrium not only mimics a clinical condition—that of postoperative atrial fibrillation—but offers one of the cleanest in situ tests available of Mines' hypothesis regarding reentry. As modified 30 min or longer intervals of atrial flutter and/or AF can be reproducibly elicited. This model functions as the proof-of-concept model in all atrial experiments as it will allow one to administer guided, regional therapy in the setting of a consistent model of reentry in which activation and refractoriness as well as the inducibility and persistence of the arrhythmia in the setting of a completely reproducible lesion can be tracked.

### Example 3

### Myocardial Ischemia and Infarction

Wit and Janse described the characteristics desirable in animal models of ischemia and infarct induced arrhythmias as follows: First, they should occur in hearts with a healing or healed myocardial infarct, since this is the pathophysiologic setting of clinical arrhythmias; second, ventricular premature depolarizations, VT and VF should occur spontaneously and sometimes should cause death as they do in humans; third, these arrhythmias should be initiated by the triggers that incite them in humans, including spontaneous or stimulated ventricular premature depolarizations, stress, and/or other factors that may increase sympathetic discharge; fourth, with regard to ECG and response to programmed electrical stimulation the tachyarrhythmias in the animal should resemble those in the human; finally, reproducibility of the arrhythmias is needed if new interventions are to be evaluated. With regard to these criteria, a variety of occlusion/ischemia induced models have been reported since Sidney Harris' experiments describing a two-stage ligation of the left anterior descending artery in the dog and the resultant ventricular arrhythmias. As emphasized by Wit and Janse, a key to the study of these models has been the extent to which they manifest reproducible arrhythmias and the extent to which they resemble ischemia-induced arrhythmias in human subjects. The latter is extremely important to studies exploring therapies that mightserve as an alternative to the cardioverter-defibrillator.

The model used is a variation on the Harris model, with the exception that it is studied 5-7 days after infarction. This is referred to by Wit and Janse as a healing infarction, although as they point out—the healing and remodeling of an infarct may take months or longer to be complete. Critical to this model is that even though spontaneous ventricular tachycardias (usually non-sustained) are rare at this stage, such tachycardias are readily induced by pacing. The relationship between the reentrant arrhythmia that occurs here and human disease is best seen in two observations: (1) human subjects post infarction and having non-sustained ventricular tachycardia are at increased risk for sudden death and (2) in the 1-3 years following an infarction human subjects having inducible non-sustained ventricular tachycardia may be at increased risk by as much as 80% (Buxton in Josephson, ref 52-58). Of importance in comparing the canine model to the human condition is that both manifest epicardial sparing of the infarct and the presence of a border zone through which slow conduction is seen. Both may also have an endocardial border zone with sparing of Purkinje and myocardial fibers. It should be emphasized that in epi- and endocardium the border zone is not a neatly defined area but rather is histologically complex, incorporating healthy and damaged cells. While the endocardial border zone may at times be involved in the triggering of arrhythmias it is the epicardial zone that appears most important to the occurrence of reentry, and this is the prime therapeutic target of our studies.

### Example 4

### Accelerate Conduction—Overexpression of Endogenous Connexins Via Chemical Chaperones

hMSCs and myocytes in co-culture are treated with a chemical chaperone (4 PB, 4-Phenylbutric acid, FDA approved) or zp123 (Rotigaptide) a synthetic antiarrhythmia peptide, that increases Cx43 mediated coupling. Western blot analysis and RT-PCR will be used to test protein abundance and expression for Cx43, 40, 45. Dual whole cell patch clamp will be used to determine the time course of junctional coupling.

For zp123 two approaches will be used to assess whether it is effective extracellularly or intracellularly. Besides exposure of cells to zp 123 via the media a cDNA will be made and zp123 will be transfected into hMSCs, model cells and myocytes with peptide proteases present in the media to eliminate transfer of peptide via the extracellular space and assess its effects on junctional conductance using dual whole cell patch and connexin abundance using Western blot.

The rational behind this example is that hMSC based cellular delivery is dependent on the ability of hMSCs to couple with myocytes. Novel methods to accelerate coupling between hMSCs and myocytes are employed.

Western blot analysis and RT-PCR to test for protein abundance and expression for Cx43, 40, 45. The effects of 4PB and zp123 on hMSCs alone and myocytes alone will be assesed. Co-culturing of hMSCS and myocytes (atrial or ventricular) will also be done and a vital dye (Valiunas et al., 2005) will be used on hMSCs so that after defined time intervals of co-culture fluorescence activated cell sorting can be used to isolate the cell types and Western blot analysis and RT-PCR can be performed to assess abundance and expression respectively. A peptidase, such as dipeptidyl-peptidase or equivalent, will be used to cleave any zp123 that is secreted into the extracellular space. Dual whole cell patch clamp will be used to determine the time course of junctional coupling over a 96 hour interval at 24, 48 and 96 hours respectively. Initial results indicate that 4PB causes up-regulation. It is expected that zp 123 will cause the same up-regulation. This should translate into enhanced coupling in the form of increased junctional conductance relative to controls.

### Example 5

### Accelerate Conduction—Overexpression of Endogenous Connexins Via Inhibition of MMP-7

The effects of Matrix Metalloproteinase-7 (MMP-7) inhibition using Gefitinib, a cancer therapy drug recently shown to inhibit the action of MMP-7, on junctional conductance in myocytes is determined using dual whole cell patch clamp. The effects of MMP-7 inhibition on Cx43/Cx40/Cx45 expression and protein abundance is also determined. MMP-7 has been shown to be associated with a 53% reduction in Cx43 protein levels, cleavage of the C-terminus of Cx43, and reduced conduction velocity in infarcts induced in wild type mice but not in MMP-7 knock out mice. The effects of MMP-7 on Cx43/Cx40/CX45 expression and protein abundance is determined. It is believed that inhibition of MMP-7 will result in elevated expression of CX43 and enhanced coupling between myocyte pairs, model cell pairs and hMSCs under normal conditions.

Dual whole cell patch clamp in the presence of Gefitinib at the following dosages: 0.1, 1, 10, and 100 uM. These are in the range of dosages used clinically. Exposure times of 1, 12, 24, 48, and 96 hours are also used. Western blots will be used to monitor the abundance of Cx43 in cell exposed to drug and controls (no drug exposure). Exposure of myocytes to Gefitinib for hours to days may prove to short a time interval relative to the ability to maintain myocytes in culture. If this is the case, then model cell lines expressing Cx43 (N2A, HeLa) will be used and exposure time will be increased to weeks.

Inhibition of Getitinib is anticipated to result in increased junctional conductance as well as expression.

### Example 6

### Accelerated Conduction—Expression of Exogenous Connexins Cx46 and Cx32

Cx46 is transfected into myocytes to determine if the presence of a connexin that is able to form heteromeric channels with Cx43 and Cx40 can enhance coupling between myocytes. pH sensitivity using CO2 perfusion is tested to determine if heteromeric forms of Cx46 and Cx43 and Cx40 are resistant to acidification. It is believed that a more robust coupling between myocytes would result and would possess similar pH and voltage sensitivities to controls.

A nucleotide encoding Cx32, which is only weakly pH sensitive, is transfected into myocytes. A dual whole cell patch clamp experiment is performed and the pH sensitivity by CO2 perfusion is determined. Cx32 does not form heteromeric channels with cardiac connexins and therefore represents an independent channel population better able to function in ischemia induced acidification of myocytes. It is believed that a more robust coupling between a myocyte that is less pH sensitive than controls will result.

Cx46 is transfected into hMSCs and a time course of coupling between hMSCs and myocytes is determined. 4PB/zp123 is used in Cx46 transfected hMSCs to further enhance gap junctional membrane coupling. The time course of coupling with myocytes is tested.

Dual whole cell patch clamp to measure junctional conductance will be used in two populations of neonatal rat myocyte cell pairs or canine myocyte cell pairs. The initial experiments will use transient transfections of Cx32 or Cx46 and compare them with myocytes transfected with empty vector. There will therefore be four populations of cell pairs for each connexin. In each case, dual whole cell patch clamps is performed on cell pairs 24 hours, 48 hours, 96 hours in each population. For every experiment junctional currents will be collected at pH=7.1, followed by the bubbling of 100% CO2 to lower intracellular pH for time intervals of 1-3 minutes followed by a return to neutral pH (7.1). This method has been used in the past (Valiunas et al., 2002). A Western blot is done to test for the presence of Cx32 or Cx46 and Cx43. A pH sensitive fluorescent probe is used to allow correlation between intracellular pH and junctional conductance (see preliminary results).

The same pH sensitivity as controls for Cx46 is expected but because of the ability of Cx46 to form heteromeric channel, the possibility of significantly enhanced coupling between myocytes is anticipated.

With regard to CX32, the data should reveal myocyte cell to cell coupling is resistant to pH induced uncoupling such that at pH levels as low as 6.2 significant coupling will still be present between myocytes transfected with Cx32 and be negligible between control cells.

Standard transfection methods are used for the introduction of Cx32 into myocytes (lipid). Model cell lines transfected with Cx43 (N2A or HeLa) will alos be used and the effects of pH induced uncoupling as an alternative to the myocytes will be determined. Cx32 or Cx46 will be over-expressed in myocytes using adenovirus or AAV (see Aim 2 for discussion of the two viral vectors) and compared to over-expression of the native Cx43. Cultures will be grown on the MEA and conduction velocity obtained as described below. The experiment will be repeated in the presence of low pH to determine the impact of acidosis, and the corresponding reduction of any gap junctional conductance, on conduction velocity. This protocol will be conducted in non-transfected cultures, cultures exposed to a GFP expressing adenovirus as an infection control, and cultures exposed to an Cx32 or Cx46 expressing adenovirus. A Cx43 virus also will be employed to determine the advantage of over-expressing non-native isoforms compared to simply over-expressing the native Cx43 isoform. A rat Cx43 expressing adenovirus and an adenovirus that expresses a dominant negative form of rat Cx43 have been obtained and these will be used to study the effect of both increasing and decreasing the amount of Cx43 based cell coupling. The effect of over-expressing any Cx isoform is likely to be modest under control conditions where conduction velocity is normal. However, depending on the isoform expressed, a benefit under conditions of reduction conduction is anticipated. For example, Cx32 over-expression may be modest in cultures recorded in normal physiologic solution, but the pH resistance of this isoform will help maintain a higher conduction velocity in the presence of low pH than for cultures not expressing this channel. These experiments will initially be done using the 900 μm spacing MEAs so that a record over a larger area of culture can be made. To gain more precise information on propagation, a subset of experiments under conditions where a pronounced effect is seen (e.g. low pH in GFP vs Cx32 cultures) will be repeated using arrays with 200 μm spacing. It will be determined if Cx over-expression is protective in a low pH environment, and if increased conductance using a large conductance but pH sensitive channel (Cx46) or over-expression of the native Cx43 isoform is as effective as that using a pH resistant channel (Cx32). In other experiments the efficacy of each of these isoforms will be determined to preserve normal conduction velocity under conditions of reduced Na channel density (mimicked by TTX) or membrane depolarization (K depolarization, Ba).

In separate experiments electroporation will be used to transfect hMSCs with these constructs and determine the effect on myocyte conduction. In these experiments, a monolayer myocyte culture will be formed on the MEA and then overlayed with hMSCs on top. hMSCs expressing Cx32, Cs43 or Cx46 will be compared to those expressing only GFP to determine if the presence of additional Cx channels in coupled hMSCs can enhance conduction velocity.

These experiments again will be done in normal and low pH and under conditions of reduced Na current and membrane depolarization, as described above. In these experiments, effects on conduction velocity will be related to effects on myocytes-hMSC cell coupling.

The primary parameter being measured is conduction velocity, and to see an impact on this parameter a relatively high level of expression of any added gene will be necessary. This requires either a viral delivery or expression with MSCs.

FIG. 33a shows the time course of pH uncoupling for a canine ventricular myocyte pair. The voltage delivered by one cell of the pair is 10 mV, insufficient to trigger voltage dependent channel closure. The arrow indicates the onset of perfusion with 100% CO2 bubbled saline (pH=6.2). The right-hand panel of A shows a similar experiment using HeLa cells expressing Cx43 where the duration of CO2 exposure was just under 90s where upon the cells were perfused with normal saline. Longer CO2 exposures often result in poor recovery with reperfusion. FIG. 33c is a fluorescent image of a canine ventricular myocyte cell pair imaged during the bubbling of 100% CO2. The fluorescence intensity of a pH sensitive probe (carboxyfluorescein) was monitored and the change in intensity vs time while perfusing 100% CO2 while simultaneously monitoring junctional conductance. FIG. 33b shows a fluorescent image while FIG. 33c shows the data from the experiment in FIG. 33b where junctional conductance and fluorescent intensity are plotted vs time during exposure to 100% CO2.

### Example 7

### Accelerated Conduction—Increase Availability of I_{NA}

The SkM1 (SCN4a) sodium channel or a specific mutant (SCN4a-G1306E) of that channel is used to enhance conduction by providing a less inactivated channel at depolarized potentials. As a first step towards determining potential global use of the SkM1 construct, isolated canine ventricular myocytes were infected with an adenovirus carrying the SkM1 gene (or the mutant gene of interest) as done with the HCN2 gene previously (Qu, J., Barbuti, A., Protas, L., Santoro, B., Cohen, I. S., and Robinson, R. B. (2001). HCN2 overexpression in newborn and adult ventricular myocytes. Distinct effects on gating and excitability. Circ. Res. 89, e8-e14). After allowing 48 hours for expression the myocyte was voltage clamped at room temperature in low Na external solution (10 mM). Standard protocols will be used to determine the inactivation versus voltage curve, the activation as a function of voltage, the time constants of the activation variable m and the inactivation variable h. We will use TTX subtraction to determine the steady state “window current.” A determination of the time and voltage dependence of slow inactivation of sodium current is also made since a major concern when adding a new sodium current is that it might deliver a larger “persistent” current which could predispose to long Q-T syndrome (Marban, E. (2002). Cardiac channelopathies. Nature 415, 213-218). The results of these studies will be compared to myocytes transfected with a virus containing just GFP. The action potential will also be studied. The maximum rate of rise will be determined as a function of the holding potential or while the cell is depolarized by various concentrations of extracellular potassium (4, 8, 16, and 24 mM). Other action potential parameters like action potential duration at 50 and 90% repolarization will also be determined as a means of assessing possible effects of persistent Na current. These experiments will also compare myocytes infected with the SkM1 sodium channel gene or the mutant gene to those transfected with just GFP. Each of these properties will be determined over a number of cycle lengths (2 seconds, 1 second, 0.5 seconds and 0.33 seconds) and with a two pulse protocol with a cycle that intercalates an extra beat. To assess this Na channel approach for focal cellular a two model cell delivery system, both stably transfected with the genes of interest, will be used. N2A cells expressing Cx43 or human mesenchymal stem cells (hMSCs). The cells will be transfected with the SkM1 sodium channel, the mutant sodium channel or GFP. The sodium current will be characterized in these stable cell lines to confirm the biophysical properties are similar to those previously reported. The model cells will be co-cultured with isolated ventricular myocytes and observed for a myocyte and a non-myocyte cell in close apposition. A dual whole cell patch clamp technique and the same protocols to study the aggregate sodium current will be used. In each cell pair, one of the two cells is randomly selected to voltage clamp and leave the other in current clamp mode. The same protocols are then performed, switching which cell is in current clamp and which is being voltage clamped. The junctional conductance are simultaneously monitored. In this manner one can determine the aggregate sodium channel properties as a function of gap junctional conductance. Finally, the same action potential studies described above will be performed with an aim towards determining whether the two cell functional syncytium can generate action potentials of reasonable upstroke velocity at depolarized potentials at a range of physiologic cycle lengths while maintaining a suitable action potential duration.

To distinguish between the native SCN5A channel and the expressed skeletal muscle

SCN4A channel in myocyte cultures, differential pharmacological sensitivity is taken advantage of. The skeletal isoform but not the cardiac isoform is sensitive to p-conotoxin GIIIA (μ-CTX; skeletal IQ 50-140 nM), a drug previously employed in developmental studies of the sino-atrial node Na current. In addition, the cardiac isoform is relatively sensitive to block by Cd²⁺, with a K_{d} in the range of a few hundred μM, whereas the skeletal isoform K_{d} is in the range of tens of mM.

SCN4A has been successfully expressed in myocyte cultures using electroporation, an approach that is adequate for studying effects on action potential parameters and to characterize the expressed current biophysically within myocytes. However, to achieve the high efficiency of expression needed to impact propagation, SCN4A will be over-expressed in myocyte monolayer cultures by adenovirus infection. Using adenovirus expression of channels in neonatal myocyte cultures it was found that expression efficiency exceeds 90% at the typical m.o.i. (20-30) employed. Efficiency of expression will be confirmed for these channels by measurement of μ-CTX sensitive Na current in a random sampling of cells (because of the size of the SCN4A insert, it is not practical to combine channel and GFP marker expression within a single adenovirus).

Cultures are grown on the multi-electrode array (MEA) and then transferred to the recording apparatus where superfusion with 35° C. physiologic solution is maintained during the experiment. The culture is paced at a constant rate of 2 Hz (to exceed the typical spontaneous rate) from a large electrode embedded in the MEA at a location 2 mm beyond the recording array, and electrical activity from all 60 electrodes recorded for 20 sec. From these data, conduction velocity along the axis of propagation (average data from 3 successive stimuli) is calculated using custom software. The experiment will be repeated in the presence of elevated K (10 mM or higher) to determine the impact of membrane depolarization, and the corresponding inactivation of Na current, on conduction velocity. This protocol will be conducted in non-transfected cultures, cultures exposed to a GFP expressing adenovirus as an infection control, and cultures exposed to an SCN4A expressing adenovirus. While the effect of SCN4A over-expression may be modest in cultures recorded in normal physiologic solution, it is expected that the positive position of the SCN4A inactivation relation will serve to maintain a higher conduction velocity in the presence of elevated K than for cultures not expressing this channel. These experiments will initially be done using large spacing MEAs that were custom fabricated, with 900 μm spacing between electrodes, so that a record can be made over a larger area of the culture. To gain more precise information on propagation, a subset of experiments under conditions where a pronounced effect is seen (e.g. high K in GFP vs SCN4A cultures) will be repeated using arrays with 200 μm spacing.

Past experience with HCN2 adenovirus has indicated that lower m.o.i. does not markedly affect the expressed current density, but primarily affects the percentage of expressing cells. Therefore, the m.o.i. of the SCN4A adenovirus will vary to modulate the uniformity of expression and therefore the magnitude of effect on the maximum upstroke velocity of the action potential in myocytes within the syncytium. Parallel experiments will be carried out on cells grown on glass coverslips under equivalent conditions, and these preparations will then be fixed for immunocytochemistry to determine the uniformity of expression.

The preceding experiments will be done in cultures uniformly expressing the SCN4A construct. In a separate series of experiments custom chambers, developed for use with the 900 μm MEAs, will be used. These chambers allow growth of cells in up to 3 contiguous regions. Cells expressing SCN4A are plated in the center, between regions expressing only GFP and the transition of propagation into and out of the SCN4A expressing region is studied. Similar experiments, with 2 regions, using the 200 μm MEA are conducted for more detailed mapping.

These experiments all involve over-expression of SCN4A within the myocytes. In separate experiments electroporation will be used to transfect hMSCs with these constructs and determine the effect on myocyte conduction. In these experiments a monolayer myocyte culture is formed on the MEA and then overlayed with the hMSCs on top at different densities to achieve different magnitudes of contributing exogenous current. hMSCs expressing SCN4A will be compared to those expressing only GFP to determine if the presence of additional Na channels in coupled hMSCs can enhance conduction velocity. These experiments again will be done in normal and elevated K, and with both uniform plating and regional plating of the hMSCs. Parallel experiments will be carried out on co-cultures grown on glass coverslips under equivalent conditions, and these preparations will then be fixed for immunocytochemistry to determine the heterogeneity of non-myocyte distribution.

As a control in all the experiments described above, μ-CTX is used to inhibit the expressed SCN4A channels without affecting native SCN5A channels, and confirm that the cultures then function similarly to GFP-expressing control cultures or co-cultures.

In other experiments, an adenovirus is used to deliver an siRNA of SCN5A to the myocytes, to reduce Na current and conduction velocity without altering membrane potential. SCN4A will be expressed in coupled hMSCs and the efficacy in restoring normal conduction velocity when the Na channel is in the coupled hMSC will be determined. The over-expression will be done both globally, by overlaying hMSCs on the entire monolayer, and regionally, by co-culturing only in the central portion of a 3-compartment chamber (with myocytes alone in the other 2 compartments)

To gain mechanistic insight into the effects observed in the above studies on conduction velocity, additional studies will be conducted in single cells or cell clusters to obtain information on channel biophysical properties and action potential parameters, respectively. In these experiments, monolayer cultures, in which SCN4A is expressed via adenovirus, are resuspended and replated to provide single cells or small clusters of cells for acute studies 6 hours after resuspension. The action potential studies are typically carried out either on the monolayers or on small clusters of 2-4 cells, since single cells are not required for these recordings. The monolayers are stimulated with an extracellular electrode while the cluster is stimulated through the whole cell patch electrode in current clamp mode and the action potential recorded (stimulation frequency 1-2 Hz). A brief (˜1 ms) stimulus is employed to avoid distortion of the action potential upstroke. Rₘ, Vₘₐₓ, action potential amplitude (Amp) and action potential duration at 50% repolarization (APD₅₀) will be measured. Experiments will be done in both normal and elevated K, and repeated in the presence and absence of μ-CTX to define the contribution of the expressed channel. The voltage clamp studies will be conducted on single cells following resuspension. In myocytes expressing SCN4A, the I-V relation and steady-state inactivation relation will be determined. In addition, the percent of cells expressing the SCN4A channel, the average Na current density in these cells, and the relative contribution of SCN5A and SCN4A to the total Na current at hyperpolarized potentials (maximal availability) will be determined. These experiments will be done under conditions where the native cardiac Na channel is blocked by Cd²⁺, and also under conditions where the expressed Na channel is blocked by μ-CTX, so that each can be separately characterized. These data will allow one to determine the magnitude of available Na current under different expression conditions for different degrees of membrane depolarization, and relate this to measured conduction velocity under the same conditions. The native Na channel in these myocytes using whole cell recording methods has previously been studied.

A tight correlation between the fractional Na channel availability (endogenous and expressed) and conduction velocity is anticipated. Efficacy of SCN4A based therapy is dependent on both the level of expression achieved and on the inactivation relation being relatively positive when the channels are expressed within a coupled hMSC or within a myocyte, and in particular when the myocyte is diseased and possibly depolarized. However, a 1994 paper (Ji S, Sun W, George A L, Jr., Horn R, Barchi R L. Voltage-dependent regulation of modal gating in the rat SkM1 sodium channel expressed in Xenopus oocytes. J Gen Physiol 1994; 104(4):625-43) reported that this channel exhibited two gating modes, and that when held at a less polarized potential the channel favored a mode with a more negative inactivation relation. Such a phenomenon might limit the effectiveness of expressed SCN4A in depolarized myocytes. However, this same study reported that co-expression of the Na channel betal subunit shifted the channels into the mode with a more depolarized inactivation relation, independent of holding potential. Thus, the expressed channel may favor the depolarized inactivation position when expressed in myocytes that contain endogenous betal subunit. Our preliminary data suggest inactivation is positive in the neonatal myocytes, but it is possible the same will not be true in the diseased heart. Alternatively, we may find that the proposed point mutation (SCN4A-G1306E) will exhibit a markedly positive inactivation relation regardless of the holding potential, or a sufficiently positive inactivation even at depolarized potentials. In addition, SCN4a is reported to have a somewhat more negative activation relation than the cardiac isoform, which could lead to excess window current. However, chimeric SCN4A/SCN5A channels have been described that exhibit the typical cardiac activation relation but a positive shift in the inactivation relation, and we have obtained the clone of one of these chimeric channels from Dr. Bennett. Finally, we have previously studied neuronal Na channel isoforms. The neuronal isoforms (SCN1A-SCN3A) exhibit more positive inactivation relations than the cardiac isoform. Among these isoforms, SCN2A has a midpoint of inactivation very close to that of SCN4A and could serve as an alternative channel for this therapeutic approach.

### Example 8

### Accelerated Conduction—Increase Membrane Potential

In this aim, one attempts to hyperpolarize depolarized tissue to enhance the availability of the sodium channel. This can be used for focal treatments. Three constructs are used, KiR2.1 and KiR2.2 inward rectifier genes that are expressed in heart and ERG3, which is a delayed rectifier gene with a large steady state conductance at −50 mV but not at −80 mV and so should have a larger effect on depolarized tissue than that which is fully polarized. Normal myocytes will be studied and their resting potential as a function of external [K] will be observed. The loss of K conductance will be simulated by various concentrations of external Ba which block iK1. In each case the potassium equilibrium potential both theoretically and experimentally (by looking at the reversal of a K specific current like iKr) will determined. From these experiments one can determine the potential hyperpolarization by overexpression of a K conductance. One of the three genes (Kir2.1, Kir2.2 or ERG3) will be overexpressed in the myocytes by viral infection and allow two days for expression, and then determine the resting potential as a function of K (4, 8, 16, 24 mM). The potassium equilibrium potential will be determined both theoretically and experimentally (as described above). The maximum rate of rise of the action potential and action potential duration as a function of holding potential and as a function of the same extracellular [K]'s will be determined. Because the increased diastolic K conductance might render the myocytes refractory, the current threshold for eliciting an action potential and the ability of myocytes to generate action potentials throughout a wide range of cycle lengths (0.25, 0.5, 1, 2 seconds) will be determined. Finally, these experiments will be repeated with different concentrations of Ba to simulate a reduced initial K conductance. In these experiments the resting potential will be measured, the potassium equilibrium potential determined and the rate of rise of the action potential. Since Ba has effects on delayed rectifier currents, the effects on the action potential duration would not be meaningful. An increased resting potential at a given extracellular [K] or in the presence of a fixed [Ba] would constitute success, if it also yielded an increased maximum rate of rise of the action potential and did not increase current threshold or reduce the ability to generate action potentials at physiologic cycle lengths. To test the efficacy of this approach for focal delivery either N2A cells or hMSCs will be stably transfected with one of the three genes and then the transfected cells will be cocultured with myocytes. After 48 hours in culture, the two cell functional syncytia consisting of a myocyte and a transfected cell will dual whole cell patch clamp technique will be studied. The resting potential of the cell pair as a function of external [K] and as a function of external [Ba] will be determined as described above. These results will be compared to similar experiments performed at a similar time in culture on isolated myocytes alone (results obtained above). The rate of rise of the action potential as a function of membrane potential set by voltage clamp or by changing the membrane potential with either elevation of external [K] or addition of external Ba at various concentrations will also be determined. The current threshold and ability of the two cell syncytium to initiate action potentials at the cycle lengths will be determined. Success will be determined by a more hyperpolarized membrane potential at any given external [K] or [Ba] with an elevated maximum rate of rise of the action potential. This must be accomplished without a significant increase in current threshold and with an ability to generate an action potential at all physiologic cycle lengths. Kir2.1 and/or Kir2.2 will be over-expressed and the resulting cultures will be compared to those over-expressing ERG3 and to control cultures expressing GFP. The effect on conduction velocity will be determined under control conditions and following membrane depolarization. The depolarization will be achieved by elevated K. In the case of ERG3 over-expression, the depolarization also will be achieved by either μM Ba or expression of siRNA to Kir2.1 or Kir2.2. The channels will be expressed in the myocytes (by adenovirus or AAV) or in coupled hMSCs. In separate experiments, TTX or siRNA of SCN5A will be used to partially reduce Na current, and the effectiveness of K channel over-expression to increase Na current by hyperpolarizing the cell and relieving inactivation will be determined in the setting of reduced net Na current. In the case of ERG3 expression, dofetilide will be used to reduce the current and determine if the effects are reversed (i.e. if the cultures now function like GFP expressing control cultures).

To determine if the increased K conductance near the resting potential, particularly when Kir2/1/2.2 is expressed, impacts on excitability, patterned cultures will be prepared where the K channel is only over-expressed in one region, and the ability of a paced beat to propagate into and through the region of K channel over-expression will be determined. Kir2.1/2.2 and ERG3 expressing cultures will be compared. This will be done with different levels of m.o.i. to achieve different percentages of expressing cells. Varying m.o.i. will allow us to modulate the uniformity of expression and therefore the magnitude of effect on the maximum diastolic potential of the syncytium.

Similarly, the magnitude of the effect when expressing the channels in hMSCs will be modulated by altering the density of the co-cultured stem cells. The effect of both local and global co-culturing will be determined on both conduction velocity and excitability, i.e. the ability of the signal to propagate into and through a region where myocytes are co-cultured with hMSCs expressing a K channel.

Information will be obtained on the magnitude and biophysics of expressed currents, and the impact of expression on action potential characteristics including MDP, APD50 and APD90. For ERG3 expression, it will be determined if dofetilde, by inhibiting the expressed current, reduces MDP.

### Example 9

### Slow or Block Conduction—siRNA Against Native Cardiac Sodium Channel

siRNA directed against the native cardiac sodium channel will be used to selectively reduce Na current creating bidirectional block. N2A cells and hMSCs will be used as model delivery cells. Stable cell lines will be created expressing SCN5a, SCN5a+an siRNA against SCN5A, SCN5A+a nonsense siRNA. This should aid in determining which siRNAs are effective and have an appropriate negative control. The stable cell lines will then be used with either the effective siRNA or the nonsense siRNA expressed alone and will be cocultured with N2A cells expressing SCN5A. After 4 days of coculture, the dual whole cell patch clamp technique will be used and the levels of sodium current in the N2A cells expressing the SCN5A channel and coupled to the N2A cell expressing either the sense or nonsense siRNA will be compared. These two cell pair types having a similar gap junction coupling conductance will be compared. These N2A cells are easier to use for development purposes because the hMSCs flatten out with time in culture. The next step is to couple the N2A cells to myocytes again using cells expressing either sense or nonsense siRNA. The same dual whole cell patch clamp and analysis of Na channel current density as a function of gap junctional coupling conductance will be studied. Finally hMSCs stably expressing either sense or nonsense siRNA will be cocultured with either atrial or ventricular myocytes. The same experiments will be performed to determine the effectiveness of the sense siRNA as a suppressor of myocyte sodium current. Experiments will also be performed on action potential parameters as described in aim 1(a) Success would be indicated by an inability to generate action potentials at any holding potential at cycle lengths outside the physiologic range.

### Example 10

### Slow or Block Conduction—Overexpress Cx31.9

Overexpression of Connexin 31.9 will have a dominant negative effect on gap junctional coupling in pairs of atrial or ventricular myocytes resulting in reduced gap junctional coupling secondary to formation of heteromeric channels with Cxs 40 and 43. Connexin 31.9 will be transfected into isolated myocyte pairs and dual whole cell patch clamp will be used to determine junctional conductance. If Cx31.9 is a functional dominant negative then junctional conductance will be reduced from controls. This example is the precursor to focal delivery of Cx31.9 to specific regions of the heart. If Cx31.9 alters leak conductance of myocytes via the formation of hemichannels will also be determined.

Myocytes will be isolated by means already published and transiently transfect Cx31.9 using the pIRIS2-EGFP vector. Control cells will be transfected with empty vector. Both populations will be dual whole cell patch clamps at the 24, 48 and 96 hours after transfection. Isolated cells treated similarly will also be taken and immunostaining will be performed with Cx43 (Valiunas et al., 2004) and Western Blot (Wang et al., 2006) to assess the influence of Cx31.9 on endogenous connexin abundance and expression.

If Cx31.9 forms heteromeric channels with Cx43 or Cx40 that result in a channels with lower unitary conductance and or lower open probability total junctional conductance will be reduced relative to controls. Another possibility is an affect on the trafficking of other connexins. Western blots and immunostaining will be used to assess if Cx31.9 has the ability to act as a dominant negative via reduction in the total number of functioning channels via trafficking.

Model cell lines with the same paradigm will be used if the transfection of myocytes proves inefficient. It is possible that Cx32.9 acts via an other mechanism, directly altering junctional conductance. It might affect non-junctional conductances. In this case the single whole cell voltage clamp should reveal if Cx31.9 affects non-junctional conductances.

Cx31.9/30.2 will be overexpressed in myocytes via adenovirus or AAV. Whether the expressed Cx forms heterotypic channels with native connexins in the myocytes, resulting in reduced conduction will be determined. The effects on conduction velocity will be related to the effects on conductance, as measured in myocytes-myocyte cell pairs.

### Example 11

### Increased Erp without Altering APD—hERG, hERG Mutants and MiRP1 siRNA

The aim of these experiments is to prolong refractoriness without lengthening the action potential and predisposing to long Q-T syndrome. This approach employs a number of constructs that prolong deactivation of delayed rectifier K current providing an enhanced K conductance for an extended time period at diastolic potentials following an action potential. Since the approach could be global as well as focal the first set of experiments will examine the atrial or ventricular myocytes and use adenoviruses to deliver either a mutant form of hERG (hERGK538A or L539W) with prolonged deactivation kinetics or to deliver an siRNA against MiRP1 (which should leave hERG without MiRP 1 as its beta subunit prolonging its deactivation). Both atrial and ventricular myocytes will be studied and the control will be myocytes transfected with a virus containing GFP alone. After two days, the delayed rectifier currents in all three groups will be studied. The action potential will also be studied with the same protocols as described herein. An additional protocol will be added that determines the current threshold for an intercalated beat at a number of cycle lengths (0.25 sec, 0.5 sec, 1.0 sec, 2 sec). Success will be determined by extending refractoriness without extending action potential duration. Next, N2A cells and hMSCs will be stably transfected with 1) hERG; 2) the mutant hERGs: 3) hERG coexpressed with MiRP1; 4) hERG expressed with MiRP1 and an siRNA against MiRP1 (to determine the efficacy of the siRNA against MiRP1); 5) the siRNA against MiRP1 expressed alone; and 6) a nonsense siRNA. First, these cells will be studied in isolation. The expression levels of hERG or mutant hERGs will be determined by patch clamping using standard protocols. That deactivation at diastolic potentials is slower than in those cells cotransfected with hERG/MiRP1 will be demonstrated. hERG/MiRP1 cells will then be cocultured with those cells expressing either siRNA against MiRP1 or a nonsense siRNA. The cells will be cocultured for 4 days and then studied by dual whole cell patch clamp technique. Successful transfer of the siRNA will be demonstrated by a slower deactivation at diastolic membrane potentials than observed in native HERG/'MiRP1 cells alone. Finally the 1) hERG; 2) mutant hERGs; 3) the cells expressing the siRNA against MiRP1; and 4) those expressing the nonsense siRNA will be cocultured with either atrial or ventricular myocytes. Cell pairs will be studied by dual whole cell patch clamp technique. Action potential protocols identical to those studied with viral delivery earlier discussed will be employed. A longer refractory period without a longer action potential duration will be an indicator of success. Once success is determined in experiments in N2A cells, the appropriate constructs will be stably expressed in hMSCs and cocultured with either atrial or ventricular myocytes. The same experimental protocols will be employed to determine the success of the genetically engineered hMSCs to prolong refractoriness without prolonging the action potential duration of canine atrial or ventricular myocytes

Overexpression of exogenous connexins can enhance the efficacy of siRNA transfer from delivery cell to target cell. Oligonucleotide transfer in control and hMSCs and model cell lines expressing Cx40 or Cx45 or exogenously expressed Cx46 will be measured. Cx46 manifests as a large single channel conductance and might well allow the permeation of siRNAs. Results will be compared to those for Cx43 permeability. Cx32 is excluded as it does not pass siRNA. This aim will further define the permeability characteristics of various connexins to oligonucleotides. If any connexin is more effective than Cx43 in delivering siRNA then that connexin will be overexpressed in hMSCs.

Oligonucleotide transfer between hMSCs where Cx43 or Cx40 have been up-regulated via stimulation using zp123 or 4PB will be tested.

Using overexpression of exogenous connexins to enhance the efficacy of siRNA transfer from delivery cell to target cell will be tested. Measurement of oligonucleotide transfer in control and hMSCs and model cell lines expressing Cx40 or Cx45 or exogenously expressed Cx46 or Cx37. Both Cx46 and Cx37 manifest large single channel conductances and might well allow the permeation of siRNAs. This aim will further define the permeability characteristics of various connexins to oligonucleotides. Test for oligonucleotide transfer between hMSCs where Cx43 or Cx40 have been up-regulated via stimulation using zp123 or 4PB. Rationale: Connexin43 has already been shown to allow the permeation of siRNA. This is designed to illustrate whether the other major cardiovascular connexins generate gap junction channels that are siRNA permeable. Synthetic oligonucleotides (morpholinos) will be used because the constructs do not readily hybridize and are not degraded by cellular processes (Mudziak et al, 1996; Summerton and Weller, 1997).

Cell lines (N2A, HeLa) stably expressing with Cx40, Cx43, Cx45, Cx46 and Cx37 have been created. Morpholino nucleotide constructs of the form used in Valiunas et al., (2005) to probe gap junction channel permeability have been, and will continue to be, used. Junctional conductance and transfer of fluorescently labeled probes are monitored simultaneously. The fluorescent probe used was Lucifer yellow (see Valiunas et al., 2002; Brink et al., 2006). The same method has been used to show the transfer of oligonucleotides in cell pairs expressing Cx43 (Valiunas et al., 2005). The method outlined here has proven successful in allowing the monitoring of cell to cell transfer of labeled probes while simultaneously monitoring junctional conductance. Cx43 channels are known to have open probabilities near 1 when transjunctional voltage is 0-20 mV and the single channel conductance is also well established (Brink et al., 1996; Christ and Brink 1999; Ramanan et al., 2006). Determination of junctional conductance then allows an estimate of the total number of functioning channels. Quantification of the fluorescent intensity in a cell pair over time allows determination of probe concentration in both cells of a pair and thus the permeability and selectivity of the channel for a probe relative to the major conducting ion (normally K ion) within the pipettes and cells can be determined. The simultaneous measurement of junctional conductance will be used as the standard under normal conditions in vivo which will consist of cell pairs being perfused in saline or cells will be exposed for varying time intervals to 4PB or zp123 before the application of the simultaneous measurement of junctional conductance and probe permeability. The protocol for exposure to either 4PB or zp123 will be short term exposures of 1, 2 and 4 hours or long term of 12, 24, 48 and 96 hours.

Preliminary data shows that Cx40 is able to pass an oligonucleotide 12 bases long with a slightly lesser efficiency than Cx43. This result suggests that other connexins might also be suitable as the delivery conduits between cells. All the results will be compared to those for Cx43 permeability. Cx32 is excluded as it does not pass siRNA. If any connexin is more effective than Cx43 in delivering siRNA then that connexin will be overexpressed in hMSCs. This chemical stimulus and chaperone approach will also be used to see if up-regulation results in enhanced transfer of oligionucleotides.

N2A cells transfected with connexins have a tendency to have low expression levels. An initial study using morpholinos demonstrated that transfer of a 24mer (24 bases long) was hard to determine in cell pairs with low junctional conductance. If expression and junctional conductance are not sufficient, another cell linewill be used which does not suffer from poor expression. HeLa cells are such a cell line. Transfer in hMSCs has already been demonstrated and also shown was the junctional conductance of hMSC cell pairs and that hMSCs and myocytes are sufficiently robust to allow transfer of morpholinos.

### Example 12

### Optimize Delivery Platform

In this example, the delivery platform for cell therapy is optimized. The hypothesis is that late passage hMSCs will have close to the optimal properties, which include (1) prolonged expression of the transgene, (2) expression of connexins for integration into cardiac tissue, (3) absence of proliferation, (4) absence of differentiation to other tissue types, (5) absence of rejection, and (6) absence of apoptosis. Preliminary results suggest that by passage 9 there is reduced proliferation, an absence of ability to differentiate to fat, an absence of increased apoptosis, and continued expression of connexins. HMSCs will be obtained from marrow aspirates and the following assays will be executed to determine which passage would constitute an optimal delivery system. (1) The hMSCs will be transfected by electroporation and will be selected for stable cell lines. Whether the transgene is still expressed 3 months after transfection will be determined. Two transgenes will be used, GFP for easy detection, and the SkMI sodium channel. (2) Western blots will be used for Cx 43 and Cx40 both of which were previously determined to be expressed in hMSCs (Valiunas et al. 2004). (3) A Wst-1 viability assay will be employed and BrdU incorporation will be used to determine proliferation rate (see preliminary data). (4) Differentiation to adipogenic, osteogenic and cartilaginous fates will be tested by standard kits (cambrex corp). (5) The immunogenicity will be determined with a mixed lymphocyte assay and a caspase assay will be used to determine disposition to apoptosis.

### Example 13

### General Laboratory Procedures

### A: Cell Preparations and In Vitro Gene Expression

This project uses several cell culture preparations. The main preparation is primary culture of neonatal rat ventricle cells, grown as a monolayer for cell propagation studies and as small clusters or single cells for action potential and patch clamp studies of individual currents. Experiments are conducted 4-6 days after the initial culture. In some studies CHO or HEK293 cells expressing a specific channel gene are used as a convenient system for validating a construct's functionality in a cell with minimal endogenous current to complicate analysis. Finally, for co-culture experiments, hMSCs cells expressing, a specific channel gene that are co-cultured with the neonatal ventricle cells will be used.

Gene expression in these cells is accomplished by lipofectamine, electroporation, or adenovirus, depending on the cell type and culture requirements. In addition, for studies of conduction in monolayer cultures, the myocytes are grown on an array of 60 electrodes to permit mapping of propagation. Custom chamber inserts, described in the Core, permit the growth of different cell types, or the same cell type expressing different channels, in contiguous regions of the array. This allows determination of the effect of regional alteration of channel expression or cell type on propagation.

### B. MEA Recording and Analysis

The cells are plated into fibronectin coated MEA dishes with a custom frame. Experiments are typically carried out 4-6 days after initial plating. The dish is mounted in the heated amplifier assembly on an inverted microscope, and superfused (1 ml/min) with physiologic solution (in mM: NaCl 140, KC15.4, CaCl2 1.8, MgCl2 1.0, HEPES 5, Glucose 1.0; pH 7.4). Temperature is maintained at 35° C. by preheating the solution entering the chamber under the control of a feedback circuit. To study a drug effect, recordings (spontaneous or evoked by field stimulation) are made before and during drug exposure and following washout. When studying spontaneous activity, a 10 s record is stored as a separate file. When stimulation is used, spontaneous activity is recorded 5 s before and 10 s after stimulation; the stimulation lasts 10 s with a rate of ˜1.5× spontaneous rate. There are 4 pairs of stimulating electrodes (one pair on the each side of the MEA plate) and generally stimulation is evoked from at least 2 sides of the culture in control conditions and from the same sides in the presence of drugs. The recordings are analyzed (e.g. activation times) with software (MSRack) provided by Multichannel Systems, the manufacturer of the MEA system. Additional analysis is made using an application based on MATLAB which provides local and peripheral conduction velocities, amplitude of potentials and other parameters. The program constructs color maps for activation times and for potential amplitudes.

### C: Action Potential and Patch Clamp Recording and Analysis

i. Action Potential Recording

Action potentials are recorded using a patch electrode in whole cell mode on cells superfused at 35° C. These experiments are conducted on monolayer cultures when studying control conditions or when employing viral expression, which achieves near uniform expression. For studying transfected cells, the culture is resuspended on the day of experiment and cells selected on the basis of GFP fluorescence, with the GFP being part of the channel vector or co-transfected with a separate vector. Extracellular and pipette solutions are as previously employed. The external solution contains (mM): NaCl 140, KCl 5.4, CaCl₂ 1, MgCl₂ 1, HEPES 5, glucose 10, adjusted to pH 7.4. The internal solution contains (mM): aspartic acid 130, KOH 146, NaCl10, CaCl₂ 2, EGTA 5, HEPES10, MgATP 2, adjusted to pH 7.2. An Axopatch-200B amplifier and pClamp8 software (Axon Instruments) are used for acquisition and analysis. Parameters recorded are Vₘₐₓ, MDP, action potential amplitude (AMP) and APD₅₀.

ii. Na Current Recording

For adequate voltage control, temperature is maintained at 19.0±0.5° C. and patch pipettes with resistance of 1.0 MΩ or less are used. The pipette solution has the following composition (mM): CsOH 125, aspartic acid 125, tetraethylammonium chloride 20, HEPES 10, Mg-ATP 5, EGTA 10, and phosphocreatine 3.6 (pH 7.3 with CsOH). After gigaohm seal formation and prior to patch rupture, stray capacitance is electronically nulled. Following transition to the whole-cell recording configuration, 1-2 minutes are allowed for intracellular dialysis before switching to the low Na⁺ recording solution (mM): NaCl 50, MgCl₂ 1.2, CaCl₂ 1.8, tetraethylammonium chloride 80, CsCl 5, HEPES 20, glucose 11, 4-aminopyridine 3.0, and MnCl₂ 2.0 (pH 7.3 with CsOH). With this combination of external and internal solutions, I_{Na} is of manageable size and isolated from other possible contaminating currents.

Clamp protocols are generated with an Axon digitizer controlled by PCLAMP software. The currents are filtered at 10 kHz, digitized at sampling interval 0.1 ms for whole cell currents and 0.02 ms for capacitative transients, and stored on the computer for later analysis. The membrane capacity (in pF) of each cell is measured in the Cs⁺ rich solution by integrating the area under a capacitative transient induced by a 10 mV hyperpolarizing clamp step and dividing this area by the voltage step. Current amplitude data of each cell is then normalized to its cell capacitance.

If experiments demonstrate evidence of inadequate voltage control, for example, a “threshold phenomenon” near the voltage range for sodium channel activation, and/or an inappropriately steep increase in current amplitude in the negative slope region of the I-V relationship curve, the data are discarded. For the other clamp protocols there should not be a crossover of currents as their size changes and the peak should occur at about the same time. Whole cell I_{Na} current is obtained by subtracting the traces elicited with comparable voltage steps containing no current (using prepulse or manipulating the holding potential to inactivate the sodium channels) from the raw current traces. In this way, the cell capacitance and linear leakage, if present, is subtracted. To examine the peak current density, voltage steps (40 ms duration) from holding potential (V_{H}) of −100 mV are given stepwise from −50 mV to +45 mV. Peak current at various test voltages (Vₜ) is plotted to obtain the current-voltage relationship curve. The peak current at each Vₜ is then normalized to cell capacitance (pA/pF) to obtain a current density-voltage relationship curve. “Steady state” inactivation curve is characterized by using a double-pulse protocol. Here, a 1000 ms conditioning pulse to various potentials (from −110 to −10 mV) is followed by a 1 ms interval back to V_{H} (−100 mV) before a 40 ms test pulse to peak current voltage. Each double-pulse protocol is separated by a 5 s recovery interval. From these data, the steady state inactivation curve for each cell is obtained by normalizing currents to the maximal current elicited from a conditioning potential of −110 mV. The Boltzmann equation is used to describe the data and to obtain V_{0.5} and the slope factor k.

iii. K Current Recording

Native and expressed inward rectifier current is measured in the whole cell configuration at 35° C. using a ramp or step protocol. The internal and external solutions are the same as for the action potential recording, but nifedipine (5 μM) is included to block L-type Ca current. Pipette resistance is 3-4 MΩ. The ramp protocol is from −120 mV to +20 mV (5 s) from V_{H} of −60 mV. I_{K1} current is isolated as Ba-sensitive current (Ba 4 mM). The step protocol is from −120 mV to +40 mV (holding potential −60 mV) in 10 mV increment with a duration of 400 ms.

### D. Scar-Induced AF Model:

Dogs are anesthetized with: propafol (5-7 mg/kg) and isoflurane (1-3%) and the total anesthetic time is standardized to 4 h. A right lateral thoracotomy and pericardiotomy are performed and the RA is exposed to air for 1 h. Then a 5 cm right antero-lateral atriotomy is performed using an atraumatic clamp. Tissue is removed for biophysics and biochemistry and the site is oversewn. Additional instrumentation includes RAA and 2 RA bipolar recording electrodes, linearly along the top of the scar, but 1 cm away from it. Also, one bipolar ventricular pacing electrode and one bipolar recording electrode is implanted for activation times and for ERP on the LA. All leads are exteriorized in a subcutaneous pocket. Post-surgical recordings will be made with baseline pacing at CL=300 msec and measure atrial activation times and ERP.

Three to four days later the animals are anesthetized as above and recording and pacing leads are retrieved and the femoral artery is cannulated for BP recordings. Atrial activation times and ERP and ventricular activation and ERP and BP are recorded during atrial pacing at CL=300 msec. AF is then induced with rapid pacing from LA starting at CL=ERP and ramping down to 10 msec (10 msec decrements). Pulse amplitude, duration, and CL of effective stimulus are recorded. In these experiments atrial flutter is defined as having a rate of 300-350 bpm, and a regular rhythm. AF has an average atrial activation interval=<150 msec with irregular electrogram morphology and rhythm. The arrhythmia is recorded including ECG and electrograms for 30 min and—if it doesn't cease earlier—defibrillate. After 15 min, repeat activation time and ERP measures and BP and reinduce the arrhythmia as above. If the original arrhythmia (with respect to both rhythm and duration) can not be reinduced within 4 h, the experiment is over.

i. Chronic Tachy-Pacing Induced AF

A thoracotomy is performed and the heart suspended in a pericardial cradle. A bipolar pacing lead is attached epicardially to the left atrial appendage. The bipolar lead is routed subcutaneously to an Itrel Pulse Generator and the unipolar lead to a Medtronic Kappa SR403 pacemaker, both of which are implanted in the right posterior thorax. Bipolar electrodes for pacing and recording are sewn to the RAA, LAA and the RV apical epicardium, tunneled subcutaneously to the right posterior thorax and exteriorized. The dogs then recover for 2-3 weeks during which they are laboratory trained and monitored for electrical stability.

Experiments are performed on conscious animals resting quietly on their left side. After surgery and before initiation of pacing all dogs are in sinus rhythm and the ventricles are paced at 60 bpm. Two-three weeks after recovery from surgery a control ECG and electrophysiology study are performed. ERPs are measured via the bipolar electrodes on the RAA and LAA.

Single extra-stimuli are applied at the end of a 10-beat drive train at 2× diastolic threshold, while pacing at BCL=400, 300 and 200 ms. ERP is defined as the shortest S1S2 interval producing a propagated response having the shortest A1-A2 interval and manifested on ECG as either a P- or an f-wave.

Rapid pacing is initiated at 900 bpm from the LAA. Dogs are monitored biweekly for the first week and then at weekly intervals. During each session rapid pacing is stopped and the atrial rhythm recorded. If the dog is in AF it is monitored to measure AF duration. When AF terminates spontaneously, ERP is measured and the ECG recorded. Rapid pacing is then resumed until the dogs develop chronic AF (defined as 5 days of persistent AF) or nonsustained AF (lasting 30 min-24 h). For the purpose of this study, the endpoint is the occurrence of sustained or nonsustained AF (which to date has occurred in 38 days in 100% of dogs paced at 900 bpm). Those dogs which do not fibrillate after 60 days of pacing are considered to have reached the en of the protocol.

Cells will be delivered by subendocardial (via catheter) or subepicardial injection (open chest) to interrupt propagation from the arrhythmogenic focus.

### E. Myocardial Infarct Model

Myocardial infarction is produced by a two-stage ligation of the left anterior descending coronary artery (LAD) approximately 1 cm from its origin. (Harris AS: Delayed development of ventricular ectopic rhythm following experimental coronary occlusion. Circulation 1950; 1:1318-1328) The chest is closed in layers and an airtight seal established. Dogs are studied on the 3^{rd} to the 5^{th} day post-occlusion. This experimental model was selected, rather than other models of canine VT, because reentry occurs at 3-5 days in a narrow rim of parallel-oriented myocardial fibers on the epicardial surface of the infarct. This model was predicted to be useful for determining the effects of tissue anisotropy on reentrant circuits, as has turned out to be the case.

Four-five days after coronary occlusion, the dogs are anesthetized and ventilated, the chest is opened via a median sternotomy, the pericardium opened, the heart supported in a pericardial cradle (Coromilas et al; Electrophysiological Effects of Flecainide on Anisotropic Conduction and Reentry in Infarcted Canine Hearts; Circulation. 1995; 91:2245-2263) and epicardial sock electrodes (98 electrode terminals organized in 7 strips, each harboring 2 rows of 7 electrodes separated by 1.5 cm) are sutured onto the RV and LV surface. (Jame M J, Sosunov E A, Coronel R, Opthof T, Anyukhovsky E P, de Bakker M J T, Plotnikov A N, Shlapakova I N, Danilo P Jr, Tijssen J G P, Rosen M R. Repolarization gradients in the canine left ventricle before and after induction of short-term cardiac memory. Circulation 2005; 112: 1711-1718). In addition, 24 needle electrodes (0.5 mm diameter) with terminals at depths of 1, 5, 9 and 13 mm below the epicardial surface are inserted into the LV wall and 12 needle electrodes with terminals at 1 and 5 mm below the epicardial surface are inserted into the RV wall. Because the deepest terminal in the LV sometimes records a cavity potential, the actual number of intramural recordings is usually less than 120. The electrograms and 2 surface ECGs (leads I and II) are simultaneously recorded using a personal computer-based data acquisition system, as previously described. The reference signal is derived from a virtual ground electrode connected to the mediastinum. Recordings are made during atrial pacing at 5% faster than sinus rate. Selected episodes are stored on the hard disk of the computer. Analysis is done off-line with a custom made data analysis program. (Potse M, Linnenbank A C, Grimbergen C A. Software design for analysis of multichannel intracardiac and body surface electrocardiograms. Comp Methods Progr Biomed 2004; 69: 225-236).

To induce ventricular tachycardia, programmed stimulation protocols with either single, double, or triple premature stimuli are used from each of 4 stimulation sites (LAD, base, lateral, and center). The stimulus pulse is 2 ms in duration and 2-4× diastolic threshold. Sustained monomorphic ventricular tachycardia are defined as the occurrence of repetitive complexes of ventricular origin with a uniform QRS morphology lasting longer than 30s. All sustained VT have a stable CL. Non-sustained VT is defined as runs of 3 or more repetitive complexes that terminate spontaneously before 30 s. The stimulation protocol is continued to completion even if sustained VT is initiated. If VF is repeatedly induced from a single site, stimulation is discontinued from that site and ERP not determined. (Coromilas et al; Electrophysiological Effects of Flecainide on Anisotropic Conduction and Reentry in Infarcted Canine Hearts; Circulation. 1995; 91:2245-2263).

The QRS morphology of the tachycardias is classified from 2 orthogonal ECG limb leads as follows: (1) R in which the complex is predominantly an R wave with a small or no Q wave and S wave, (2) QS in which the complex has a small or no R wave, (3) RS in which the complex has an R wave at least ¼ of the amplitude of the S wave, and (4) QR in which the complex has a Q wave at least ¼ of the amplitude of the R wave. An experiment is classified as having VT with different QRS morphologies if the morphology is different in at least one of the two leads. This classification is not quantitative and lacks a high degree of sensitivity; it may sometimes fail to distinguish between different morphologies. However, it does possess a high degree of specificity, ensuring that what is classified as different morphologies always are different morphologies. Costeas et al: Mechanisms Causing Sustained Ventricular Tachycardia With Multiple QRS Morphologies; Circulation. 1997; 96:3721-3731).

### F. Measurement of ERP

The ERP is measured at each site of stimulation during the protocol in which single premature stimuli are applied to initiate VT. The LAD stimulus site is always on the noninfarcted right ventricle. The central stimulus site is always in the middle of the epicardial border zone in or near the region of the reentrant circuit. The basal and lateral sites are sometimes in normal myocardium and sometimes in the infarct, depending on the extent of the infarct in different experiments. Premature stimuli have a strength 2× diastolic threshold, which is the same as the basic drive stimuli. The ERP is defined as the maximum S₁S₂ interval at which a conducted response is not elicited by S₂. The ERP is determined at the longest pacing CL in each experiment at which the ventricles are reliably captured. This CL ranges from 280 to 400 milliseconds. (Costeas et al: Mechanisms Causing Sustained Ventricular Tachycardia With Multiple QRS Morphologies; Circulation. 1997; 96:3721-3731)

G. Quantum Dot Identification of hMSC Localization

Although QD loading was achieved by either electroporation, lipid mediated uptake or passive incubation, passively incubating hMSCs in QD media results in nearly 100% of cells loading with a pattern that extends to the cell borders. The intracellular QD cluster distribution is uniformly cytoplasmic and largely excludes the nucleus. Populations of hMSCs are loaded with QDs by passive incubation for 24 hours, washed and replaced with fresh media. Cells are routinely passaged and as they divide, they split their cytoplasmic contents to each daughter cell, diluting the ultimate concentration of QDs in progeny over time. The presence of intracellular QDs does not affect ability of cells to overexpress genes after transfection. Hence, in all hMSC experiments the cells will be loaded as well with QDs to permit tracking of location of cells and correlating the cell locus with the electrophysiology in situ. In all gene therapy experiments GFP is coexpressed to provide tracking of constructs.

### H. Western Blot Analysis

Heart samples from dogs will be prepared for Western blot analysis as previously described (Duffy et al., 2004). Tissue pieces from subepicardial cell layers will be separated from midmyocardial layers and lysed (50 mM Tris-HCl pH 7.4, 0.25 mM Na-deoxycholate, 150 mM NaCl, 2 mM EGTA, 0.1 mM Na₃V0₄, 10 mM NaF, 1 mM PMSF, 20 ul of Complete Protease Inhibitor, (Roche Pharmaceuticals)), sonicated and incubated on ice for 30 min prior to centrifugation to remove cell debris. The samples will be diluted in 2× Lammeli buffer, run on 7.5%-12% SDS gels and electrophoretically transferred to Immobilon-Psq PVDF membrane (Millipore), and probed for 1 hr at room temperature using either polyclonal or monoclonal antibodies for the connexin or channel protein of Interest. Following rinses in PBST (PBS with 0.05% Tween20) membranes will be incubated with horseradish peroxidase-conjugated rabbit secondary IgGs (Santa Cruz Biotech, Santa Cruz, Calif.). Protein bands will be detected using Amersham ECL detection kit (Amersham Bios, Piscataway, N.J.) and exposed on Fuji X-Ray film.

### I. Immunofluorescence

Heart samples will be rapidly frozen in liquid nitrogen and sectioned using a Leica 3050S cryostat. Sections will be fixed in 4% formaldehyde for 10 min at RT, blocked (PBS+10% Goat Serum+0.4% Triton-X 100) for 1 hr at RT then incubated with primary antibodies directed against connexin proteins at 4° C. overnight. Following 30 min rinse (3×10 min, PBS+0.4% Triton-X 100) slices will be incubated with secondary antibodies (Alexa Fluors, anti-mouse 488 and anti-rabbit 595) for 1 hr at RT. Slices will be rinsed for 50 min (5×10 min), and mounted on glass microscope slides with Vectrashield anti-fade agent (Vector Laboratories, Burlingame, Calif.) and examined a on a confocal microscope (Olympus BX61WQ Fluoview 500 Confocal System). Images will be processed using Imaris Image Pro Software (Bitplane Scientific Solutions).

### J. Statistics

The statistical methods employed have been used by us and others for years. In intact animal experiments n=6 is adequate to test statistical significance parameters measured, using two way repeated-measures ANOVA. For these studies the experimental unit is the individual dog. Microelectrode and patch clamp data are tested with a nested ANOVA. Real time PCR and Western blot data are evaluated with a t-test where the design is a single comparison to control and via ANOVA as above when a temporal sequence is involved. When appropriate in ANOVA, adjustment for multiple comparisons is made using the Bonferroni test where variances are equal and Games-Howell test where variances are unequal. P<0.05 is considered significant.

### K. Vertebrate Animals

The animal model used is the adult male and female dog in experiments done after 1 to 8 weeks of surgical intervention, construct administration, and, in some experiments, cardiac pacing using pacemakers manufactured commercially (Medtronic) for long term use, and implanted under sterile surgical conditions. The dog has been selected because of its usefulness in studying and understanding the mechanisms of arrhythmias.

In contrast to rodents and despite its cost, the dog's electrophysiologic properties are sufficiently akin to those of the human that the observations can be readily referred to their application in human subjects. Existing literature is periodically searched to ensure that the dog remains the most suitable experimental animal and that there is no unnecessary duplication of experimental work. Use of the canine model eliminates any need for using primates to further test applicability of data to human subjects. Numbers of animals are included in the budget section. The animals are housed in the Institute of Comparative Medicine on BB 18 and 19, which provides the most up-to-date means for care and protection of laboratory animals. All protocols used for animal research are reviewed by the Institutional Animal Care and Use Committee before research is permitted to begin. Yearly reevaluation of protocols is provided as well by the Committee.

Care of animals is under the supervision of a full-time staff of veterinarians and veterinary technicians. Healthy dogs that are heartworm—free are purchased from selected breeders. They are housed in the animal facility and brought to the laboratory on the day of the experiment. Anesthesia for acute, non-survival surgery is intravenous pentobarbital Na or IV propafol induction followed by inhalational isoflurane. The majority of these experiments involve a cardiectomy following anesthesia. For those procedures requiring survival surgery, the procedure is done in a sterile surgical unit. Here, following intravenous propafol induction, inhalational isoflurane is used. Adjacent to the surgery suite is a recovery room that is supervised by a veterinary technician, with a veterinarian on call. Since the terminal procedure in all experiments is cardiectomy following anesthesia and performance of final physiologic measurements, there is no need for drugs for euthanasia. Pain is promptly treated with analgesics, and food and water are provided as soon as the animal's condition permits this. Any intercurrent illnesses are promptly treated. Animals are habituated to the experimental laboratory environment over the 2-3 post-surgical weeks to ensure that when the actual experiment starts they will rest quietly in the same position.

### In Vivo Experiments

### Example 14

### Reentry Resulting from an Anatomic Pathway can be Modified by Speeding Conduction or by Blocking Conduction and/or by Prolonging ERP without Altering Repolarization

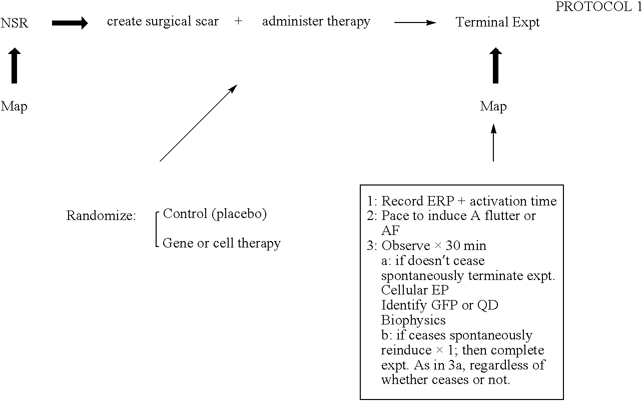

This approach uses the formation of a surgical scar in the right atrial anterior wall to facilitate pacing-induced reentrant tachycardias within three days of surgery. The tachycardia usually takes the form of atrial flutter and also can degenerate into atrial fibrillation. This is viewed as an initial test for proof of concept of each construct used before it is attempted in the more specialized models. In brief the arrhythmia in each animal is the result of a 5 cm surgical scar around which propagation circulates. Rapid RAA or LAA pacing initiates flutter. Conduction velocity, path length and refractoriness must be concordant for the arrhythmia to occur. At the time of surgery, the viral or hMSC construct is also injected. To speed conduction, Cx46, SKM1 or ERG3 will be administered, saturating the region adjacent to the scar along its entire length. To block conduction, Cx31.9 will be administered as a viral or hMSC construct to an area 2 cm×2 cm abutting on the scar at its midpoint and extending to the AV ring. The alternative approach is to use an siRNA to the NaV1.5 alpha subunit. These are expected to either block conduction or alter its path. In the latter case, a different tachycardia should supervene. To increase ERP, the ERG1 mutant or MiRP1 siRNA will be used, at the same 2×2 cm site as above. Prior to injection and to pacing, the conduction time along the path will be mapped and the ERP at each site will be determined as well. The animal will be brought back to the laboratory in the conscious state on day 3 and the inducibility of the arrhythmia as well as the ERP will be evaluated. The animal is then anesthetized and conduction is epicardially mapped. After testing ability to initiate flutter or AF via pacing, the entire scar and adjacent myocardium are removed and studied as follows: ⅓ of the 2×2 cm region perpendicular to the scar is removed for hMSC study (based on GFP or quantum dot labeling); ⅓ will be disaggregated for biophysical study of the construct that has been overexpressed; ⅓ will be placed in the tissue bath for microelectrode study of action potentials, ERP and conduction. The GFP staining or hMSC fluorescence study will be done as described by us previously in 10 uM tissue sections, the intent being to demonstrate presence of positive cells. Quantification is not relevant as ⅔ of the sample is being used for the other studies. Microelectrode experiments include pacing at CL=200-4000 msec to determine voltage/time course of repolarization, measurement of ERP at 3 CL (4000, 1000 and 500 msec); measurement of activation time at all CL. Variables recorded include MDP, amplitude, Vₘₐₓ, and APD to 30, 50 and 90% repolarization. To measure conduction, a linear region of epicardial fibers in atrium (and if available, endocardial trabeculae) will be focused on and via the use of two microelectrode impalements and a stimulating electrode, conduction velocity will be accurately determine.

Based on preliminary data and knowledge of the model it is anticipated that atrial flutter will be consistently induced and will be used to test the effects of the constructs used on reentry. Because the atria are thin-walled, adequate distribution of viral or hMSC constructs in the regions injected will be achieved. It is anticipated that Cx31.9 or NaV1.5 siRNA will effectively slow or block conduction, whereas conduction will be sped by Cx46 overexpression or by SKM1 (or G1206 mutant) or ERG3. It is believed that those constructs that slow conduction or those that speed it will themselves suffice to prevent/terminate reentry is not certain. It is anticipated that the ERG1 mutant and the MiRP1 siRNA constructs are critical in that they will prolong refractoriness without affecting repolarization and in so doing will terminate at least a subset of arrhythmias. Of greater importance is the combinations of therapies, especially the speeding of conduction and prolongation of ERP which should maximize the likelihood of antiarrhythmic efficacy.

The advantage of the additional techniques used at terminal experiments is that they will confirm (a) the delivery of construct to substrate via immunohistochemical and molecular biological techniques; (b) test the biophysical outcome of the channel construct of interest, and (c) test the cellular mechanism against that which is seen in situ.

### Example 15

### AF can be Delayed in Onset and Slowed in Evolution by Modifying the Conducting Properties of the Tissues in which the Impulses are Propagating

Two primary models will be used: (1) long-term left atrial pacing, in which improving gap junctional conductance or overexpressing novel Na channels or hyperpolarizing may bring advantage by increasing conduction velocity; and (2) left atrial appendage pacing in which conduction is slowed/blocked at the base of the LAA. This is a surrogate for a third model that is more complex and will be used only in settings where constructs prove effective in model 2. This third model is rapidly firing foci (induced by pacing) in the pulmonary veins or coronary sinus in which conduction block may be advantageous by preventing propagation of the triggering beats. Two sub-hypotheses will be tested in collaboration:

A: Globally Increasing Gap Junctional Conductance by Overexpressing Connexin46 in Atrium Will Result in Improved Inter-Atrial Conduction and Will Delay Af Evolution into a Persistent State.

This will be done by administering an AAV construct globally to the atria. The method to be used is an adaptation of that described by Hammond et al. In brief, it involves injecting the construct directly into the lumens of the coronary arteries during transient outflow occlusion of the aorta. Clearly this results in loading of the ventricles as well as the atria with the Cx46 construct. But the ventricles already have sufficient Cx43 to permit normal conduction (and even if conduction were improved in ventricle this would not impact on the atrial protocol).

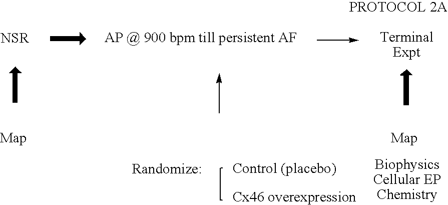

In this protocol, the atrial-pacing induced model will be used to drive animals into AF. The evolution of the arrhythmia over time will be followed in chronic dogs with 2 groups of animals (n=10/group): sham-operated controls, and those treated with an AAV vector incorporating Cx46 to induce overexpression of gap junctional proteins throughout the atria. The animals will be paced until there is first the occurrence of non-sustained AF and then sustained AF. In control, non-contact mapping will be used to determine the pathways of activation that occur in the atria. At terminal experiments, a map will again be constructed and then tissue excised from the atria for the following purposes: staining for Cx46, two-microelectrode experiments on gap junctional conductance, and action potential experiments as in protocol 1 above. Changes in channel function will be followed up by studies of mRNA and protein of the relevant channels.

### B: A Prolonged Effective Refractory Period can be Created by Slowing Deactivation of the Delayed Rectifier.

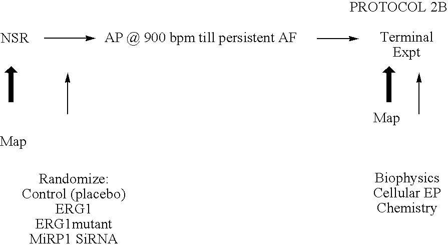

This can be achieved by (a) delivery of virus containing the ERG1 gene without coexpression of MiRP1, or (b) delivery of an siRNA to silence expression of MiRP1 or (c) delivery of a mutant form of ERG1 which has slowed deactivation kinetics (K538A or L539W). In so doing, it will be attempted to prolong ERP but not repolarization. The format of the experiments is entirely as in A, above.

It is anticipated that despite the difficulties in achieving reasonable viral construct overexpression in thick-walled chambers, adequate distribution will be achieved in the thin-walled atrium, because even if intracoronary administration fails, the method of Donahue et al. is available as a backup. Intracoronary administration will also deliver some of the construct to the ventricle. While this could be problematic in a clinical setting, this is not of concern in the present experiments whose goal is to consider actions on the atrium. Needless to say, for those approaches that appear feasible for further development, methods for delivery to atria alone will have to be further refined.

It is anticipated as well, based on results reported for rotagaptide, that increasing gap junctional conductance and conduction velocity will delay the onset and reduce the duration of episodes of AF and that prolonging refractoriness may provide a viable alternative or supplementary pathway. Finally, the immunohistochemical, molecular biological, biophysical and cellular electrophysiologic techniques used at terminal experiments will confirm the extent of transfection, modification of channel function and cellular electrophysiologic actions of the interventions.

C: Regionally Reducing Gap Junctional Conductance by Overexpressing Cx31.9 or Use of Nav1.5 Alpha Subunit siRNA Will Prevent Rapid Impulse Initiation that is Focally Triggered from Propagating Beyond its Site of Origin.

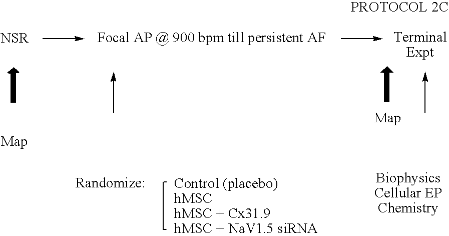

This approach is used as a more readily accessible alternative to chronic pulmonary vein pacing to test the effect of conduction block to prevent propagation of triggered impulses and thereby prevent atrial remodeling. HMSCs or AAV are used as the carrier, injecting the therapy intramyocardially. In this protocol, the use of local therapy is stressed. A construct that will slow conduction in the regions in which it is delivered is used. There will be three groups of dogs: sham controls that receive unloaded hMSCs, those treated with the hMSC construct incorporating Cx31.9 and dogs given hMSC loaded with NaV1.5 alpha subunit siRNA. All groups will be paced rapidly from the apex of the left atrial appendage; they will be brought to the laboratory weekly for an electrophysiologic study and to attempt induction of AF. Mapping will be done at the terminal experiment. At the terminal experiment, tissue will be excised from the atria for the following purposes: staining for Cx31.9, two-microelectrode experiments on gap junctional conductance, and action potential and ion channel experiments. Changes in channel function will be followed up by studies of mRNA and protein of the relevant channels.

It is anticipated that Cx31.9 or NaV1.5 alpha subunit siRNA will effectively block conduction or reduce the number pf impulses that are propagated. This is readily identified via Holter monitoring. The mapping experiments also will confirm the extent to which impulses are/are not propagated. An obvious limitation is that blocking at the base of the left atrial appendage is not the same as circumferential blocking at the point of entry of pulmonary vein into left atrium. While the cardiac vasculature can be paced both in vivo and in vitro, the ability to deliver constructs to the pulmonary vein orifice via catheter injection or hMSC-coated stents will be later explored.

### Example 16

### VT can be Delayed in Onset or Prevented from Evolving by Delivering Na or K Channel Genes to Alter Conduction and/or Refractoriness

A: Conduction can be Enhanced (and Normalized) in Depolarized Cells in a Reentrant Pathway by Altering Na Channel Availability Such that a Greater Number of Sodium Channels can be Activated From the Depolarized Membrane Potential.

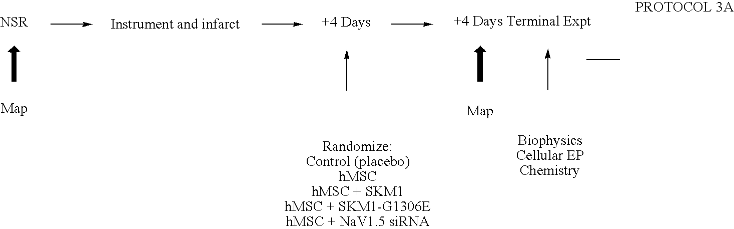

This change in biophysical properties can be elicited using hMSCs or AAV by (a) delivering hMSCs containing Na channels with a more depolarized inactivation-voltage curve.

There will be 3 groups of dogs: sham instrumented controls receiving hMSCs, those receiving hMSCs containing Na channels with a more depolarized inactivation-voltage curve (the skeletal muscle sodium channel (inactivation midpoint −65 mV), and those receiving a specific mutant of the same sodium channel (G1306E, inactivation midpoint −55 mV).

### B: Conduction can be Enhanced by Increasing Membrane Potential.

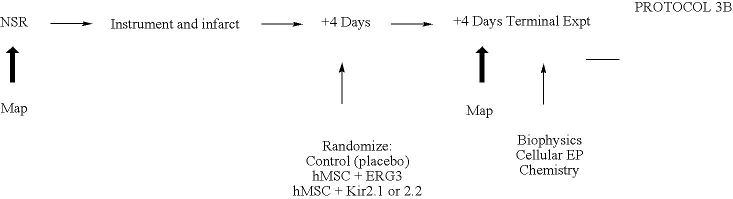

This can be achieved by delivery of hMSCs or AAV containing the ERG3 gene. Testing this hypothesis requires one study group for hMSCs and one for hMSCs plus ERG3. Kir2.1 or Kir2.2 will be used as backup.

### C: A Prolonged Effective Refractory Period can be Created by Slowing Deactivation of the Delayed Rectifier.

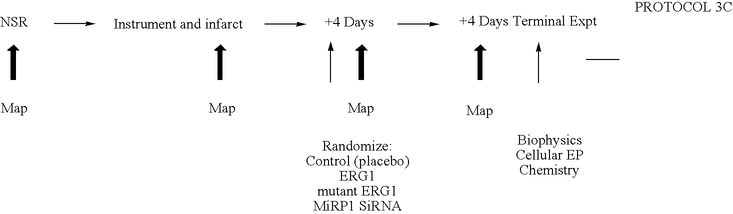

This can be achieved by (a) delivery of cells containing the hERG1 gene without coexpression of MiRP1, or (b) delivery of an hERG mutant having slowed deactivation kinetics (K538A or L539W), or c) delivery of an siRNA to silence expression of MiRP1. Prolonging ERP will be attempted such that whether there is slow conduction or conduction has been sped, interventions, refractoriness (but not repolarization) is sufficiently long to result in failed propagation. Results will be compared with simple overexpression of the hERG channel gene (Marban).

No important difficulties are anticipated in delivering viral and hMSC constructs here because the delivery site is a thin rim of viable tissue on the epicardium; there is no need to permeate the underlying infarcted region with the therapeutic intervention. It is believe that all constructs used will have an action demonstrable both in situ and in isolated tissue as altered conduction and/or refractoriness.

### Example 17

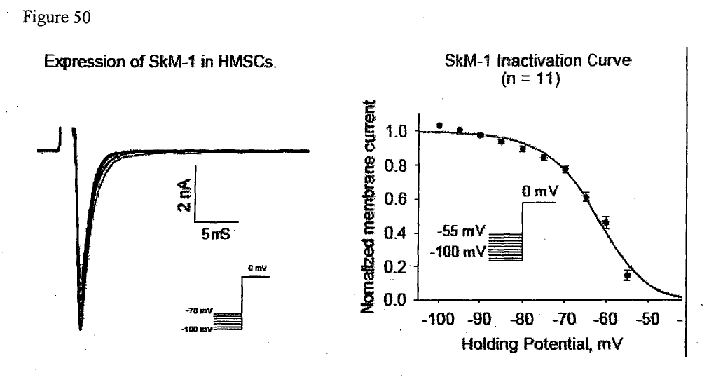

For research on myocardial infarct-induced ventricular tachycardia, a Na channel gene with a more positive inactivation versus voltage relationship than found normally in cardiac myocytes was required. The skeletal muscle Na channel was investigated because of its reported midpoint of inactivation of −68 mV. FIG. 50 shows patch clamp studies demonstrating the expression of this current in hMSCs where its midpoint of inactivation is −62 mV.

The canine atrial model was used to examine the importance of the position of the inactivation versus voltage curve of the Na channel. Since previous studies have indicated that the midpoint of the curve in canine atrium is at least −80 mV, an inactivation curve with a −80 mV midpoint was substituted into the model while carefully maintaining the rate of rise of the action potential and its shape as in the initial published simulations. Next, the same Na channel with a midpoint of inactivation of −65 mV was used in the model. These simulations were performed for propagated action potentials at various ratios of the two inactivation curves (Table 4). The higher the proportion of the conductance that has a depolarized inactivation curve, the greater is the simulated atrial conduction velocity. The effect of this depolarized inactivation curve is greatest when the diastolic membrane potential is depolarized. This could occur in the border zone of an infarct or in response to high frequencies of stimulation. Therefore the atrial model was used to examine conduction at frequencies from 1 to 6 Hz (Table 4). While propagation fails at high frequencies with an inactivation curve whose midpoint is −80 mV, fast conduction is maintained if half of the basal conductance has an inactivation curve whose midpoint is −65 mV. Thus the simulations provide evidence that expressing the skeletal muscle Na channel will help preserve rapid conduction in conditions that favor slow conduction or block.

TABLE 4
The conduction velocity γ(cm/s) is given for simulations with the
canine atrial model. In the first column, all of the Na conductance
had a midpoint of −80 mV for steady state inactivation. In the
second column, half of the baseline Na conductance is substituted
with an inactivation curve and recovery from inactivation curve whose
midpoint is −65 mV. In the third column the 50% of baseline Na
conductance whose inactivation midpoint is −65 mV is added to
the full baseline conductance whose inactivation midpoint is
−80 mV. The model was paced to steady state at each frequency.

| | 100(−80 | 50(−80 | 100(−80 |
|---|---|---|---|
| Frequency | mV):0(−65 mV) | mV):50(−65 mV) | mV):50(−65 mV) |
| (Hz) | γ(cm/s) | γ(cm/s) | γ(cm/s) |
| 1 | 52.9 | 66.9 | 72.2 |
| 3 | 45.7 | 65.8 | 70.0 |
| 5 | Failed to conduct* | 64.7 | 67.1 |
| 6 | Failed to conduct* | 63.7 | 65.3 |

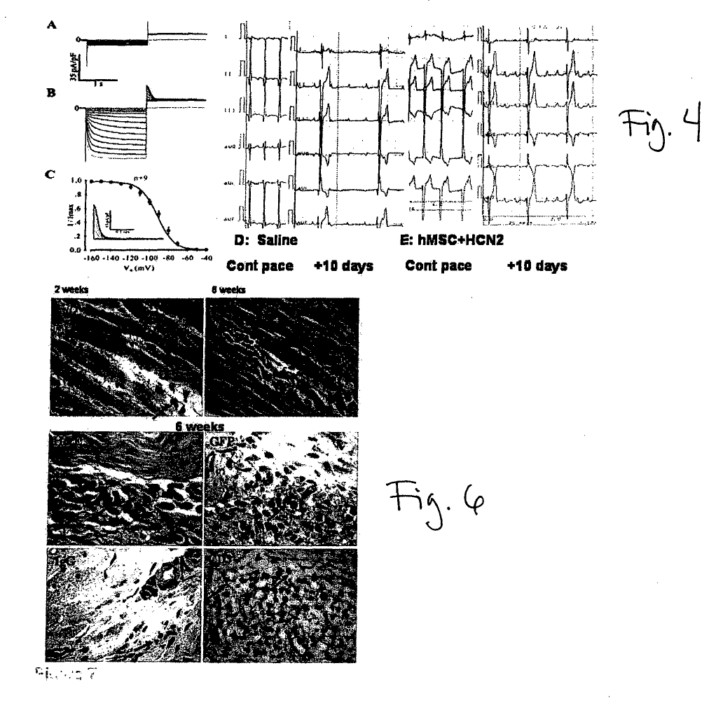

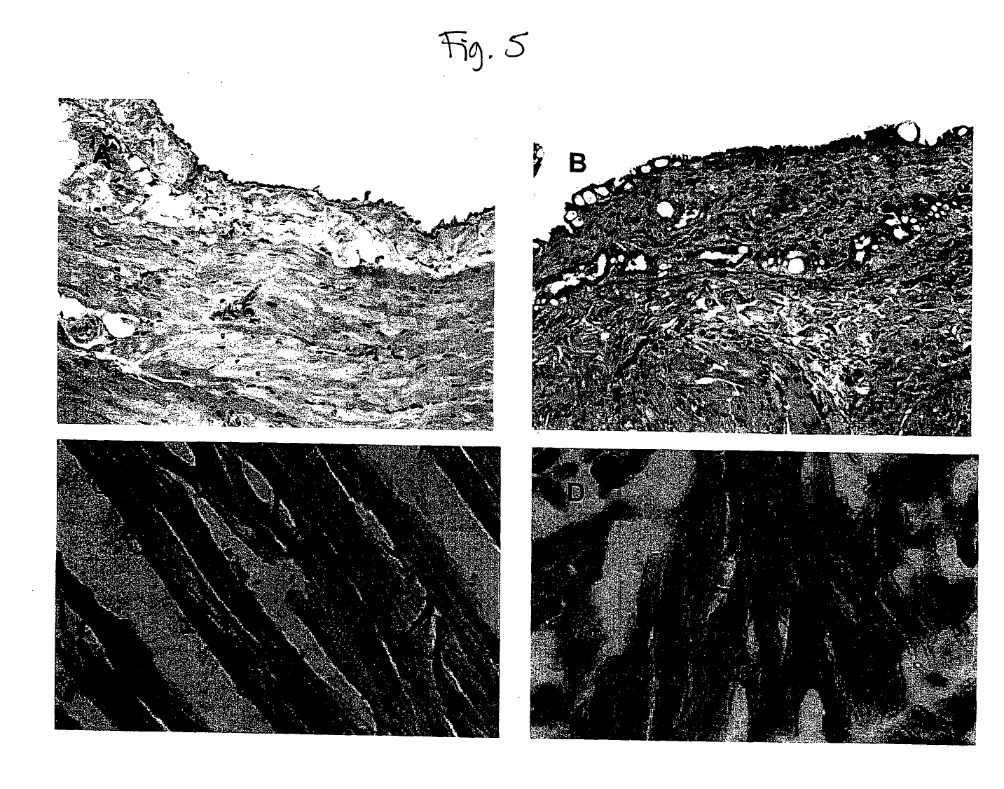

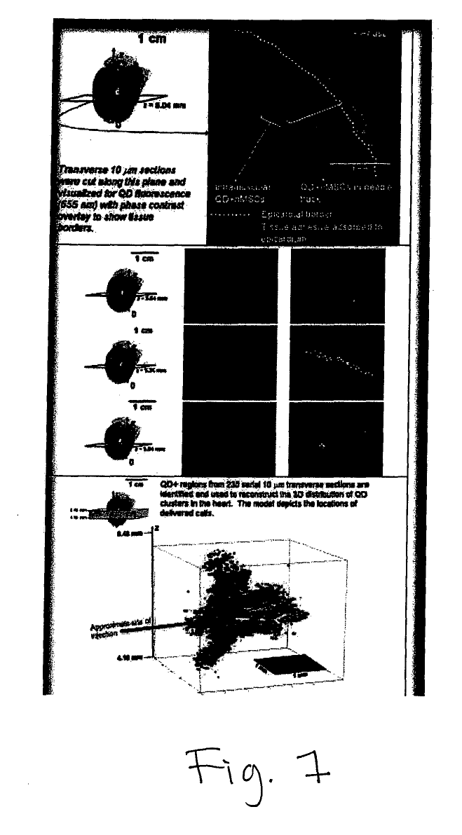

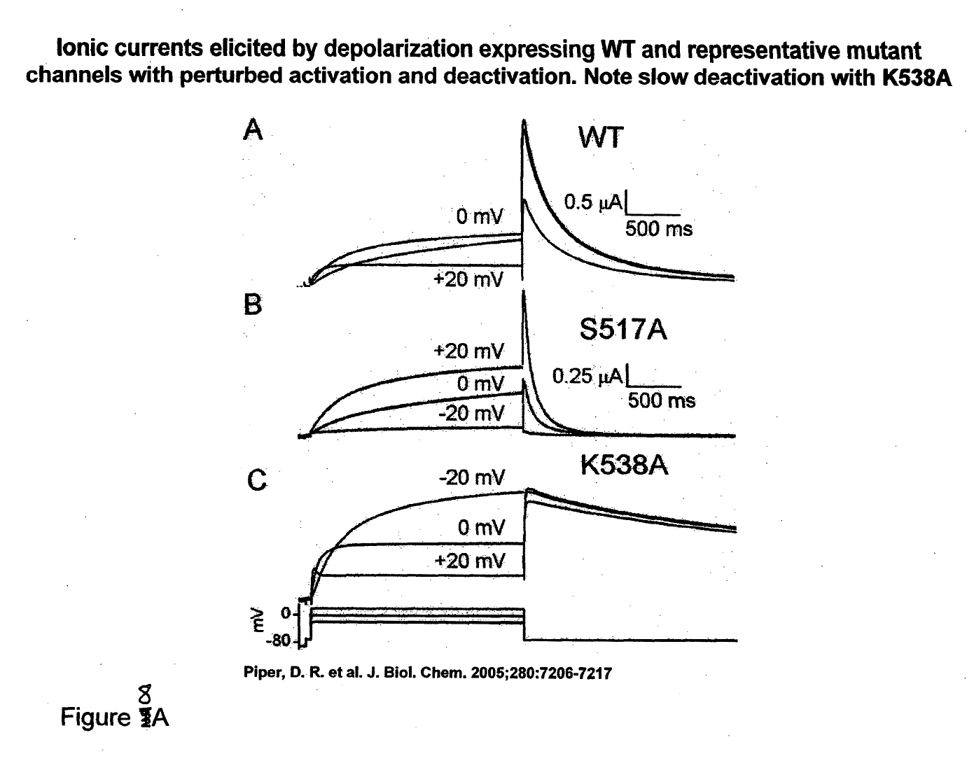

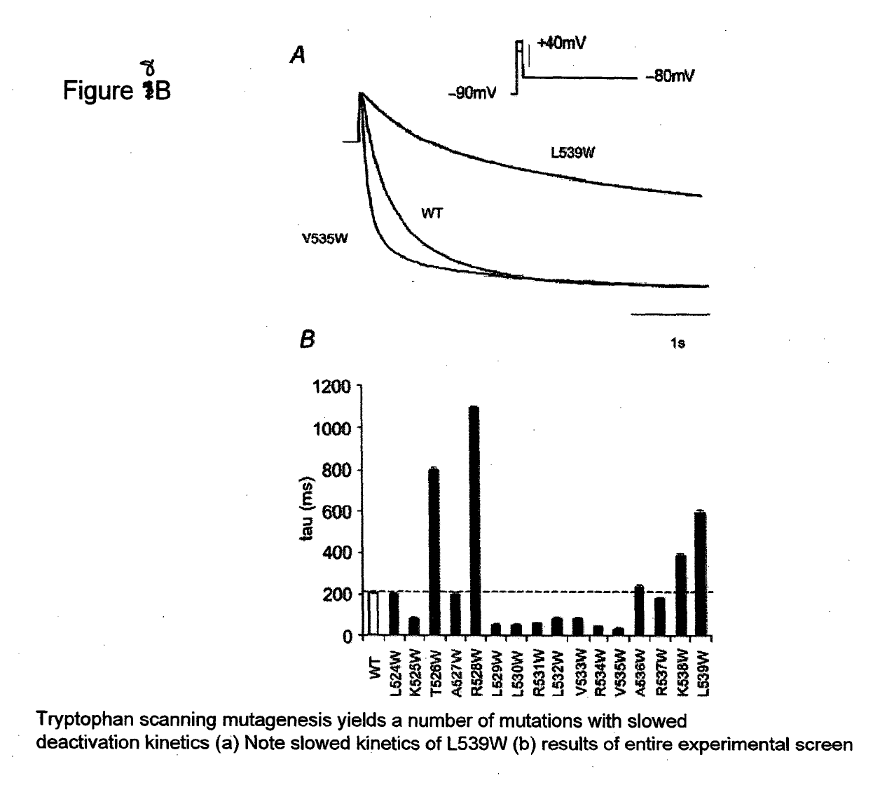

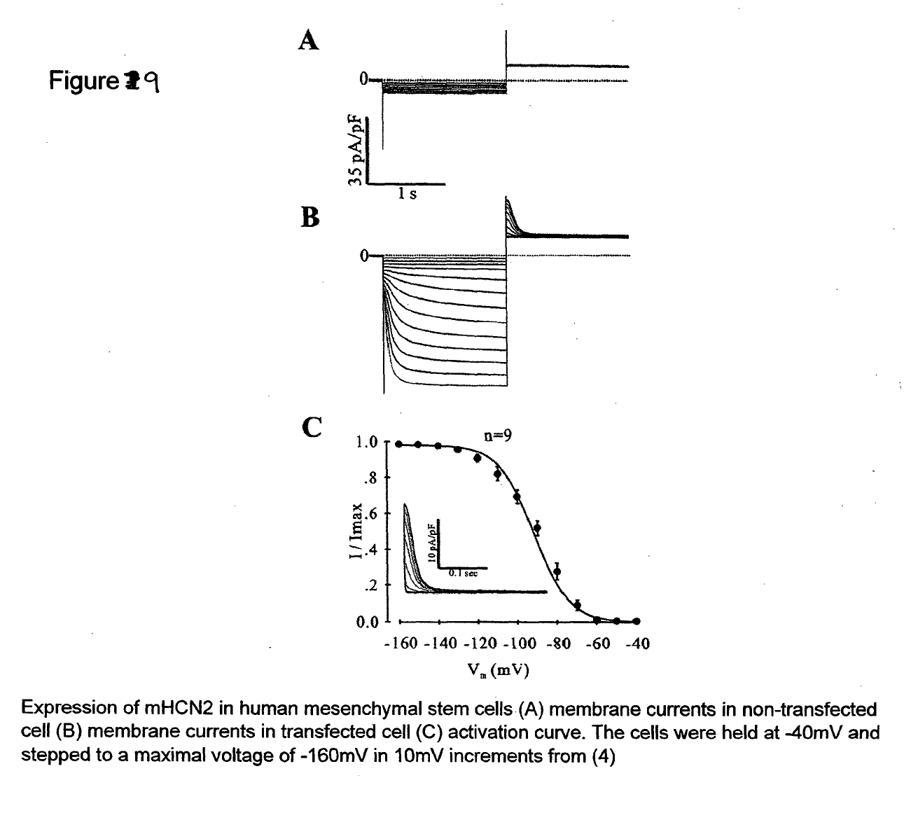

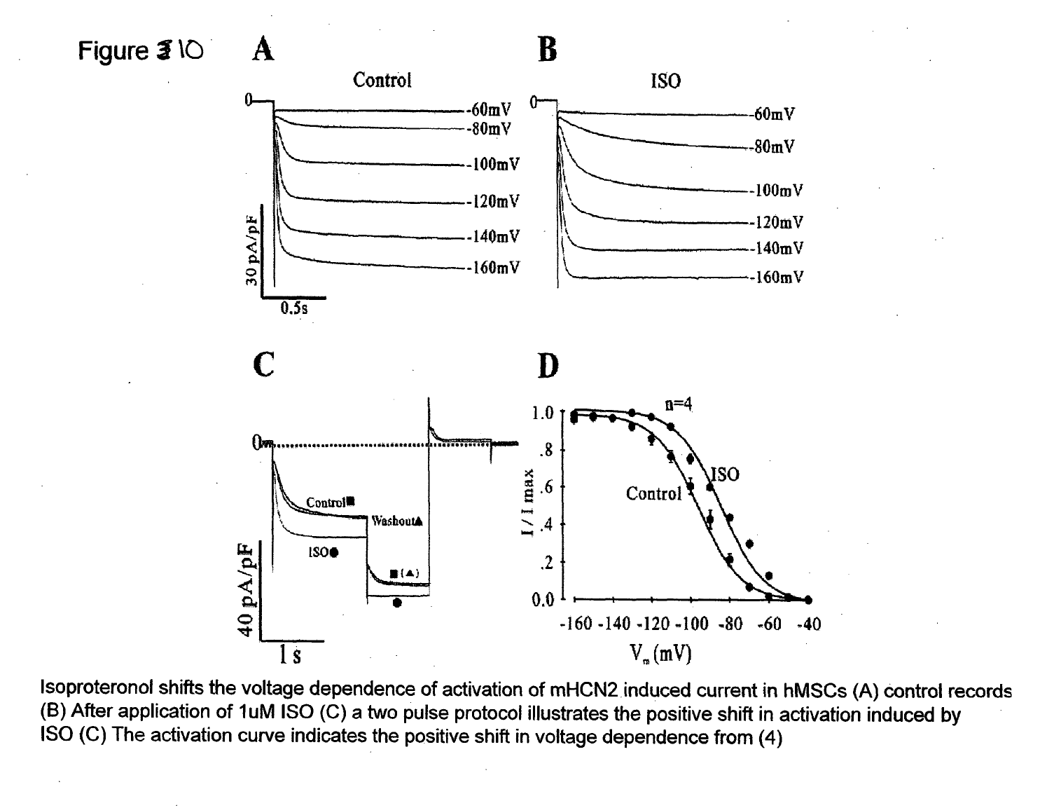

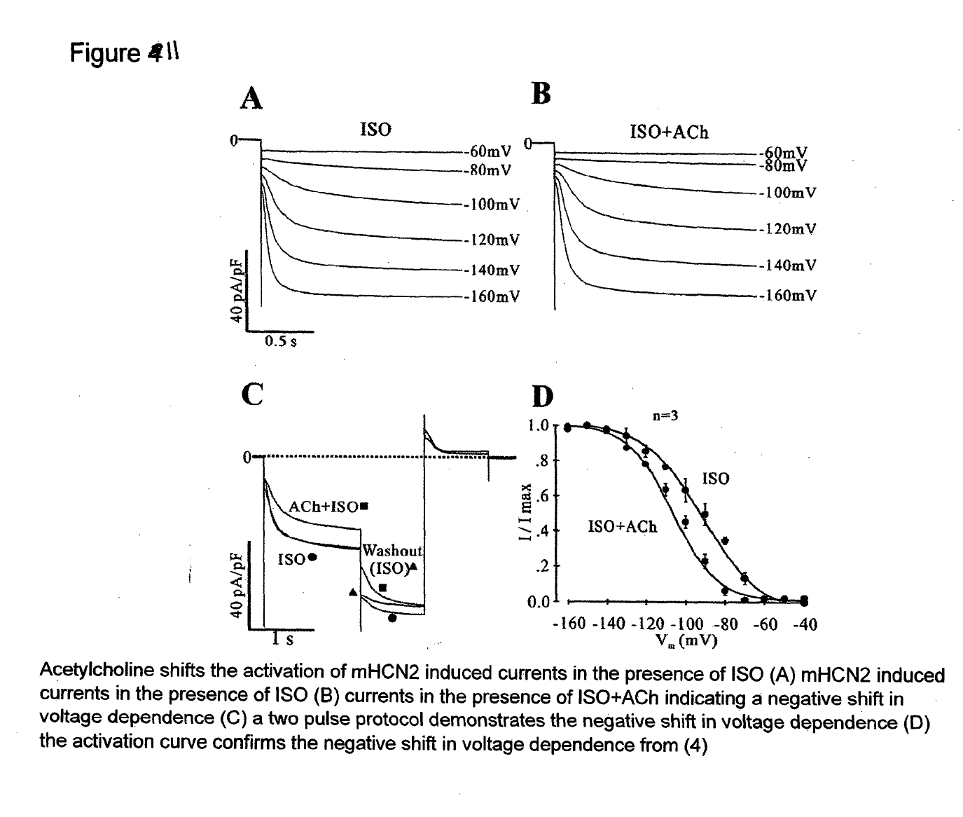

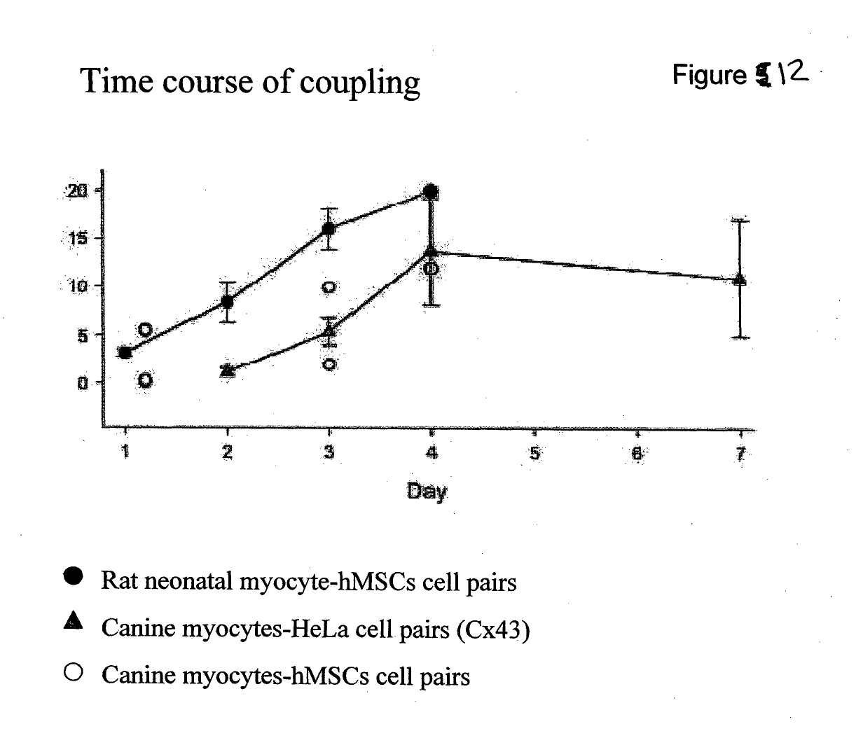

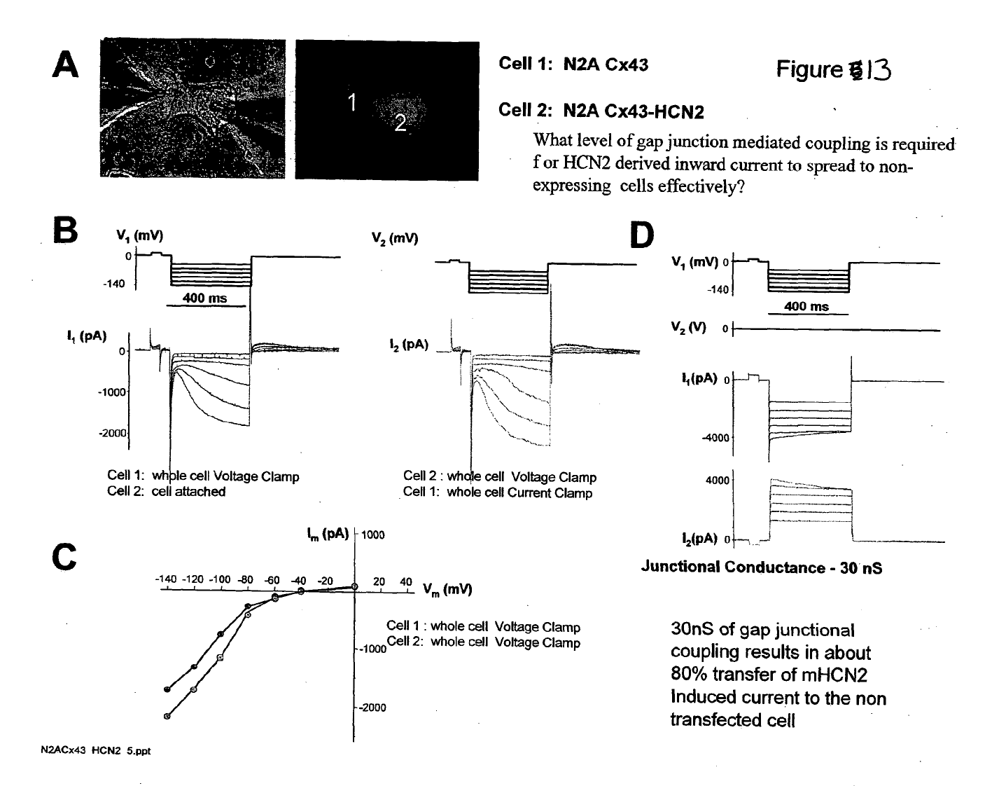

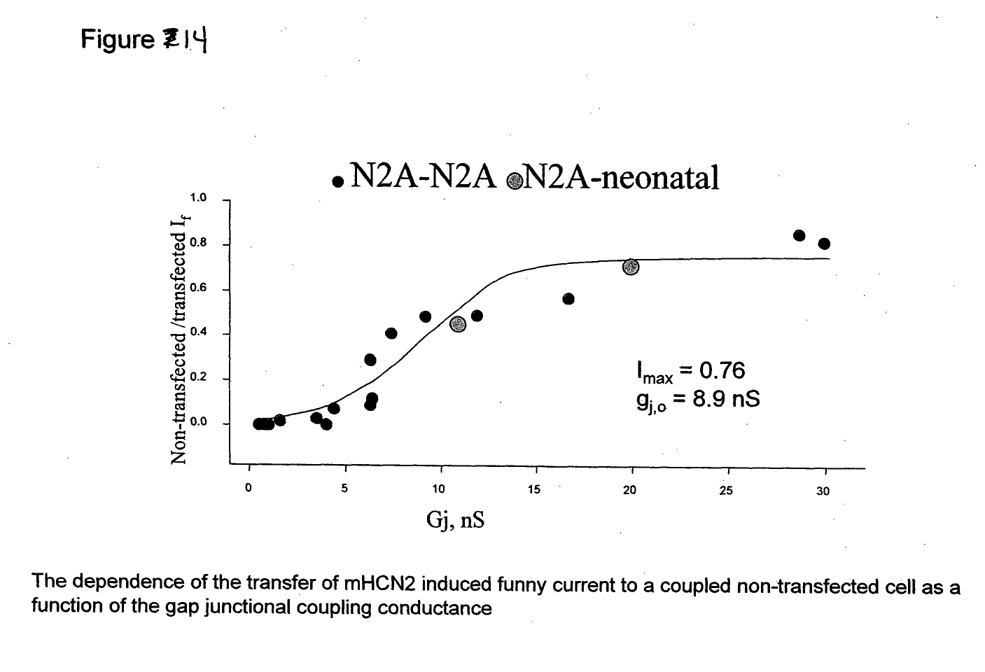

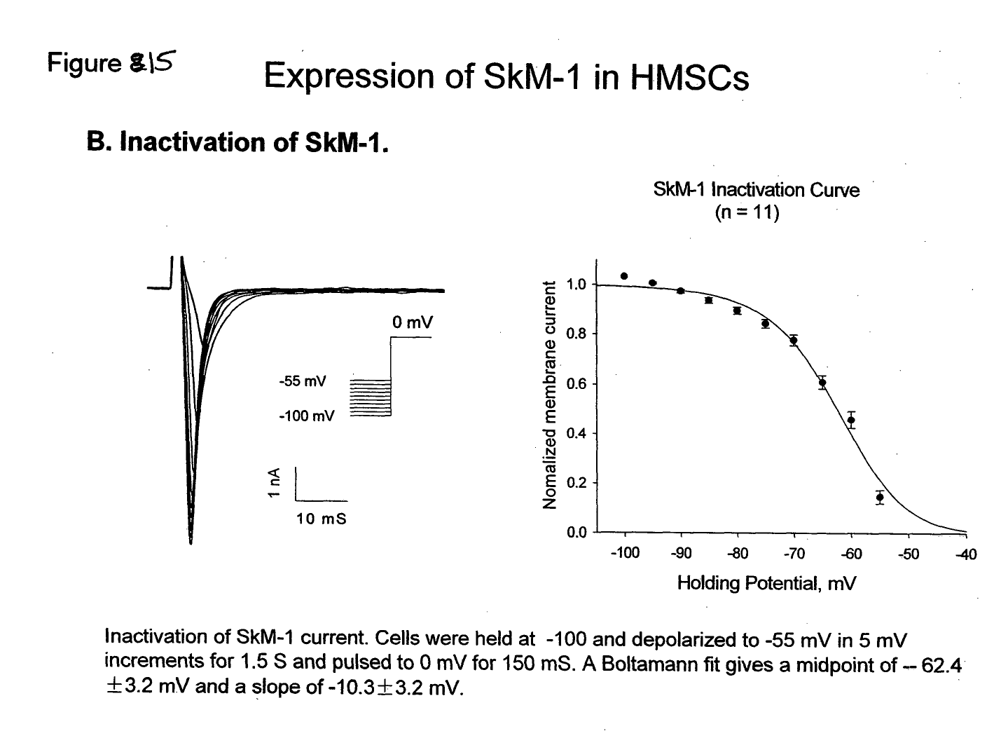

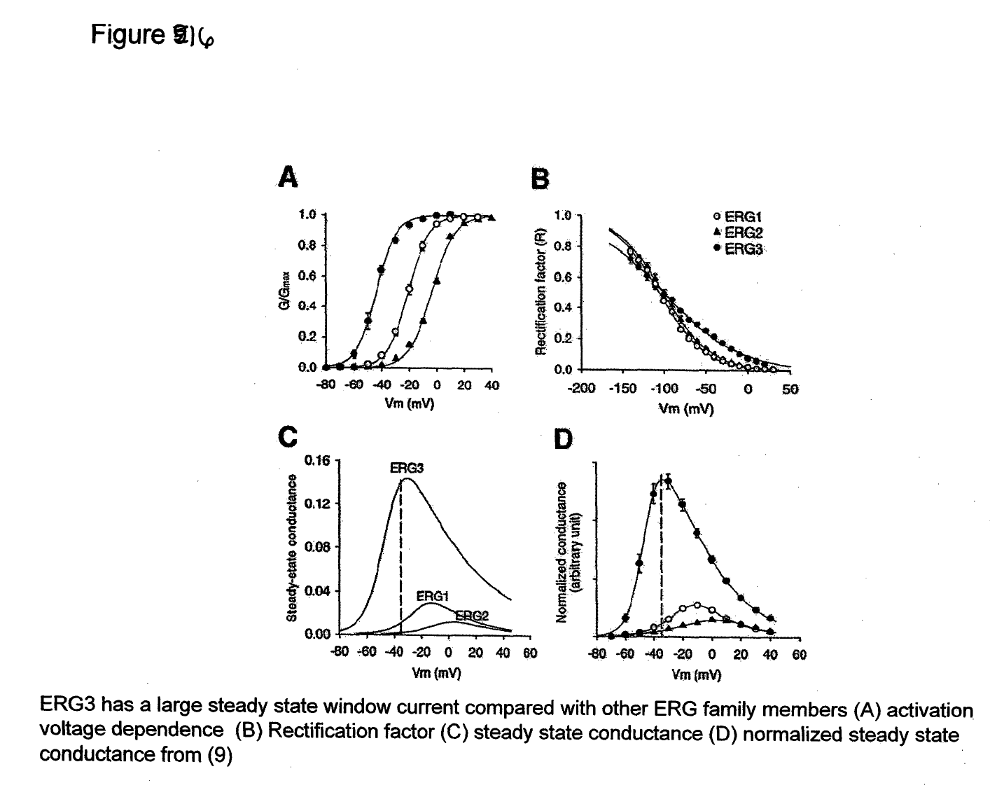

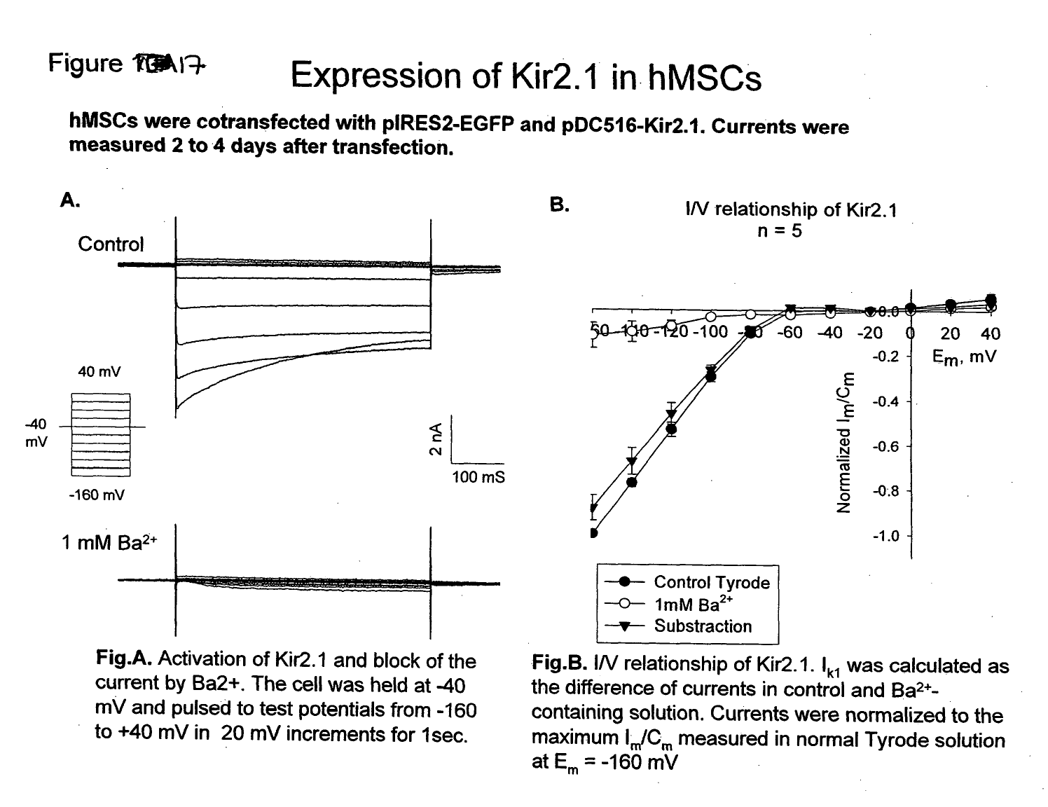

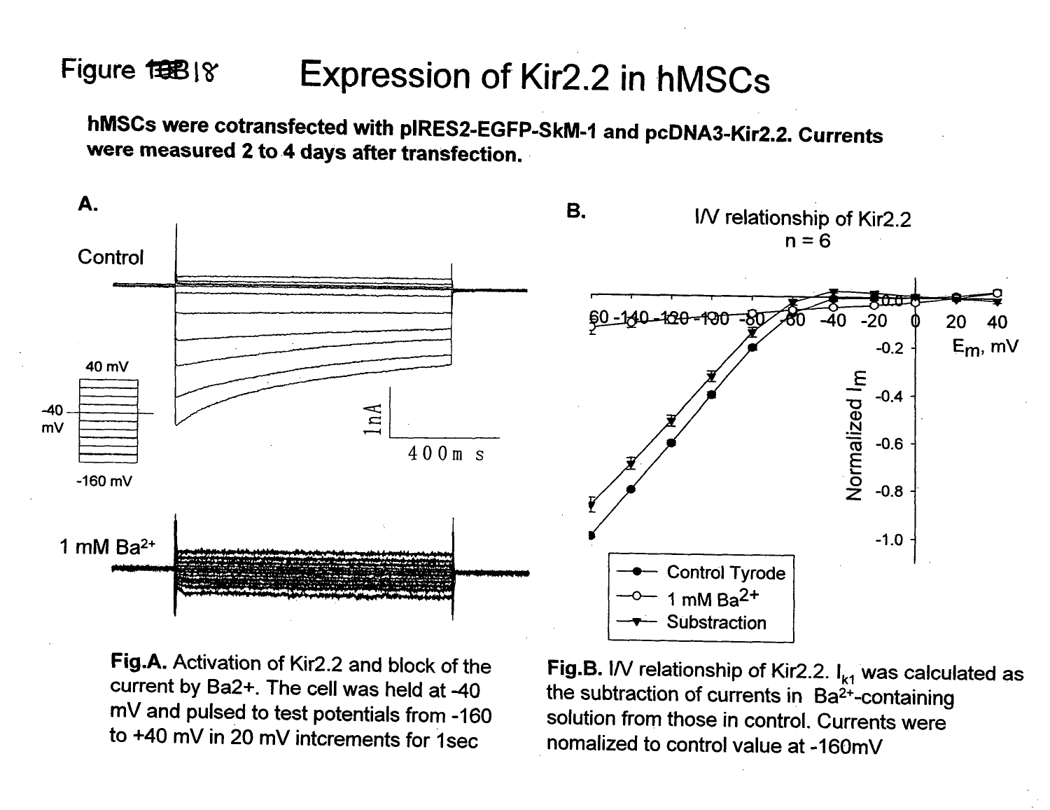

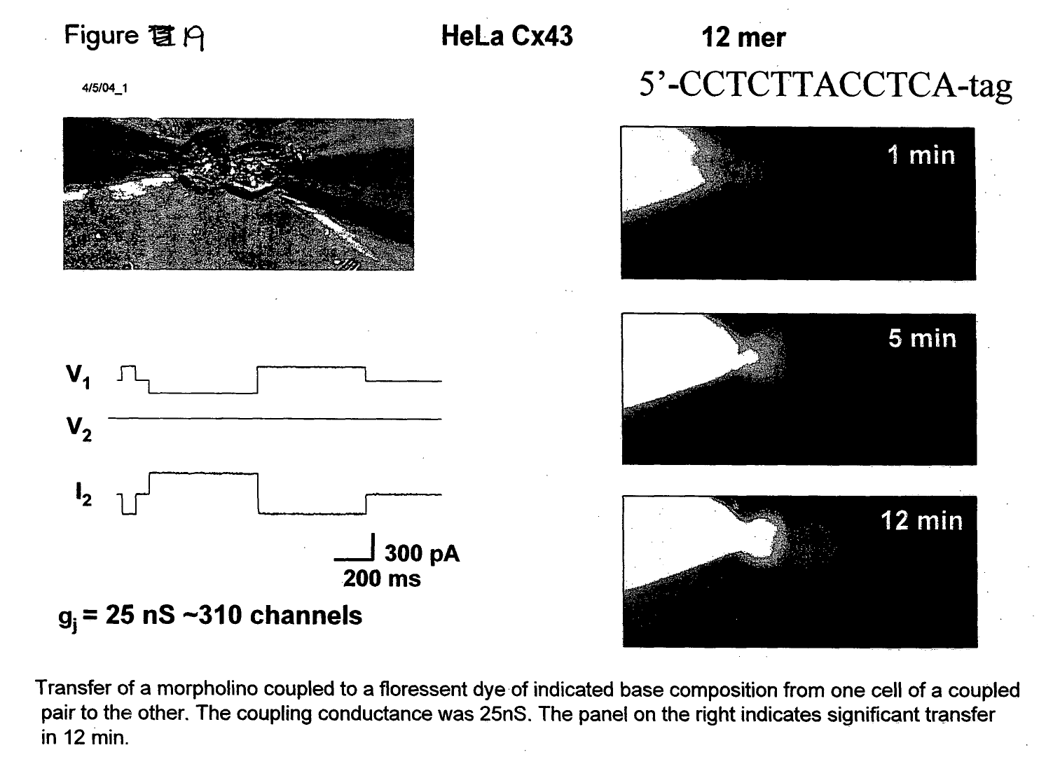

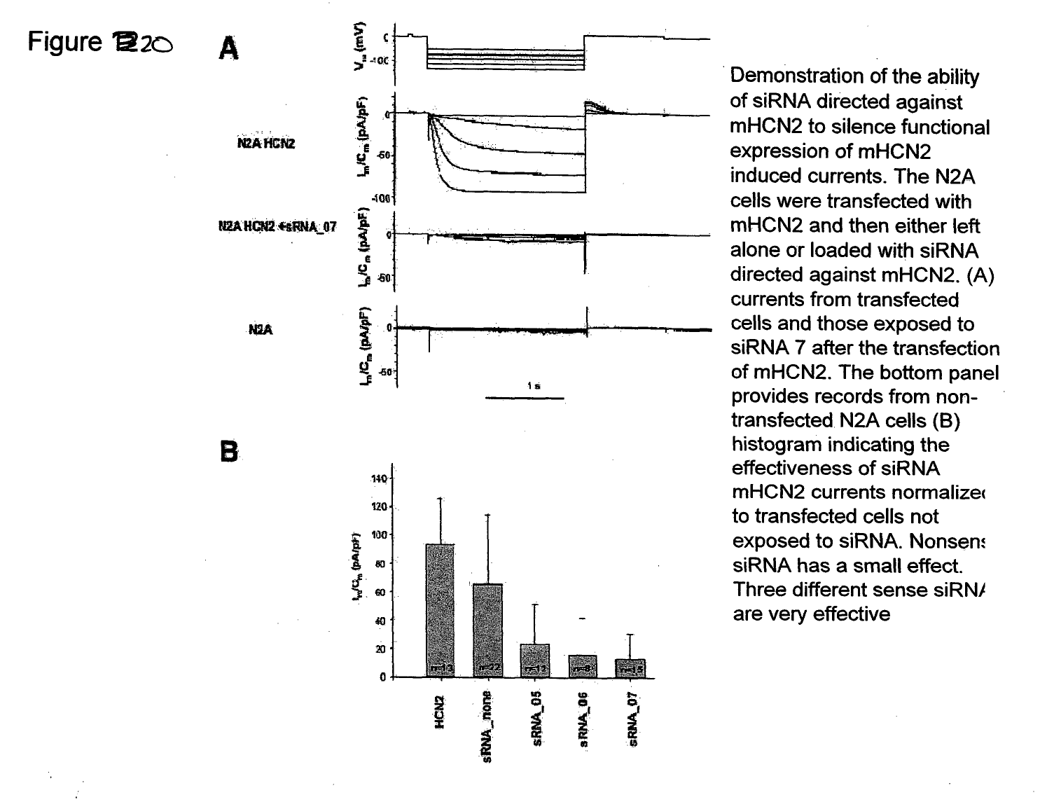

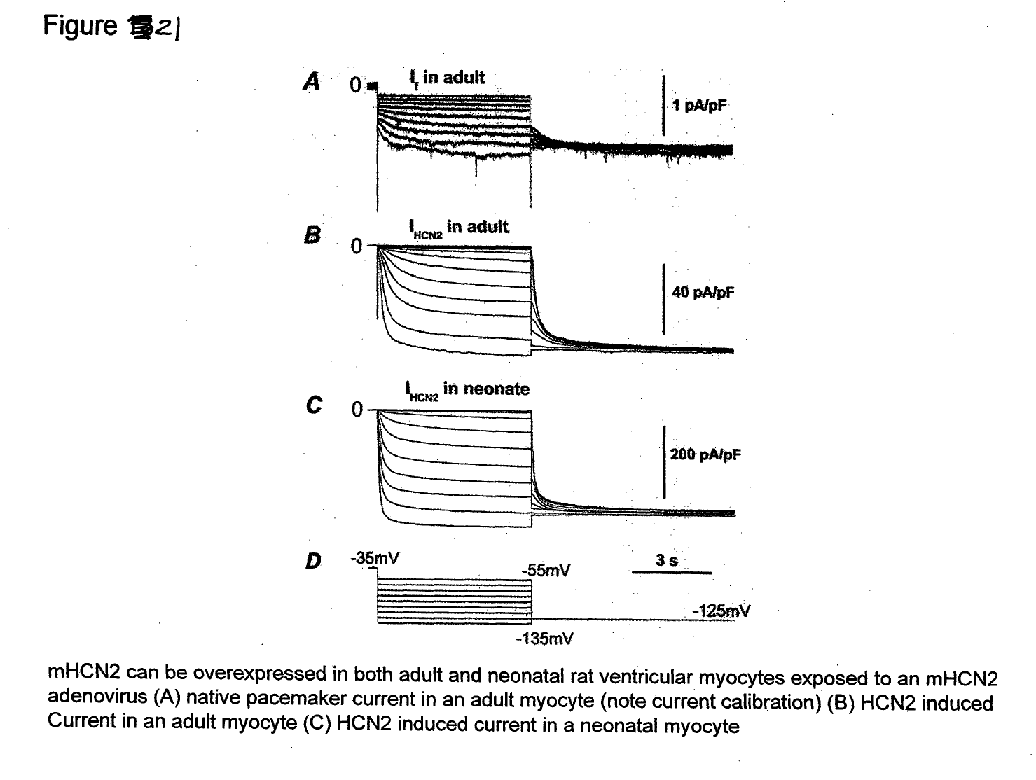

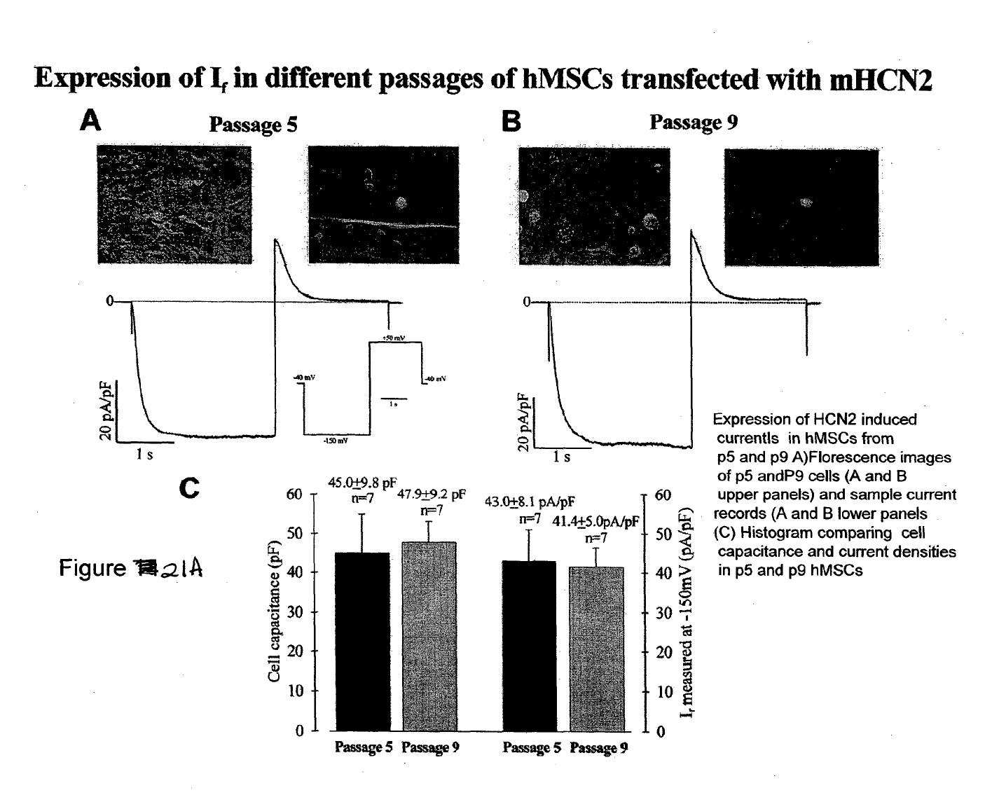

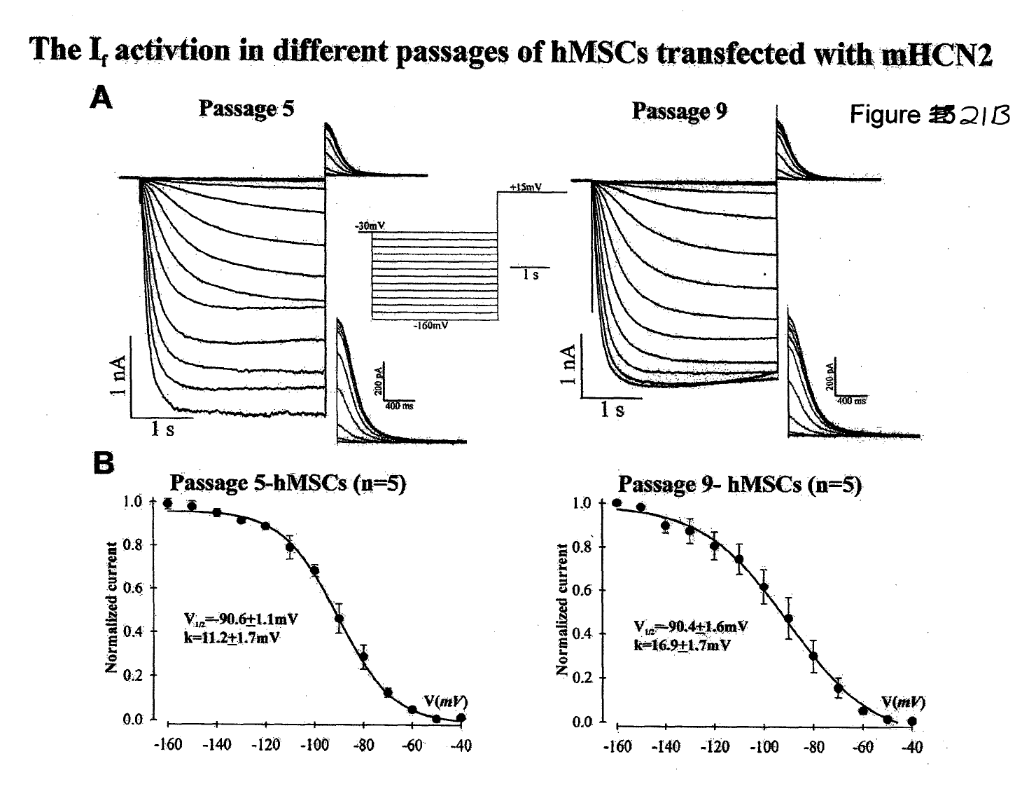

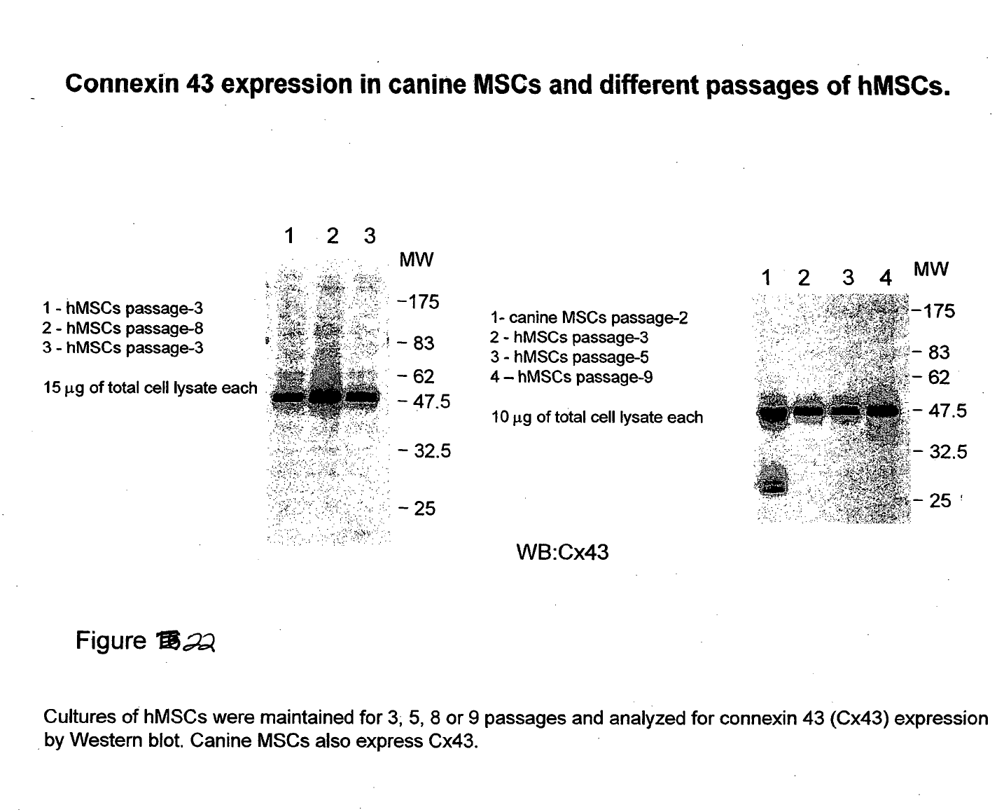

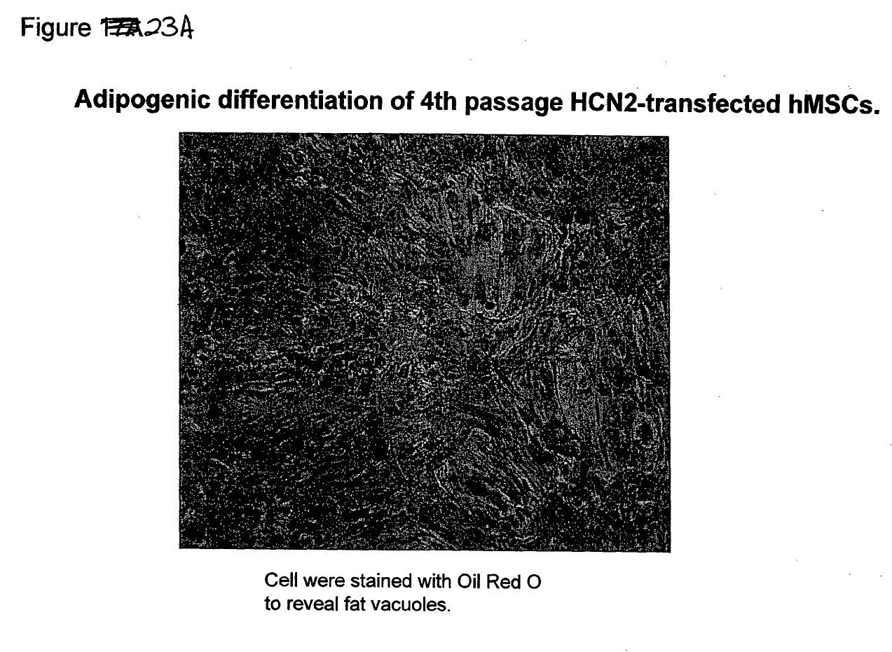

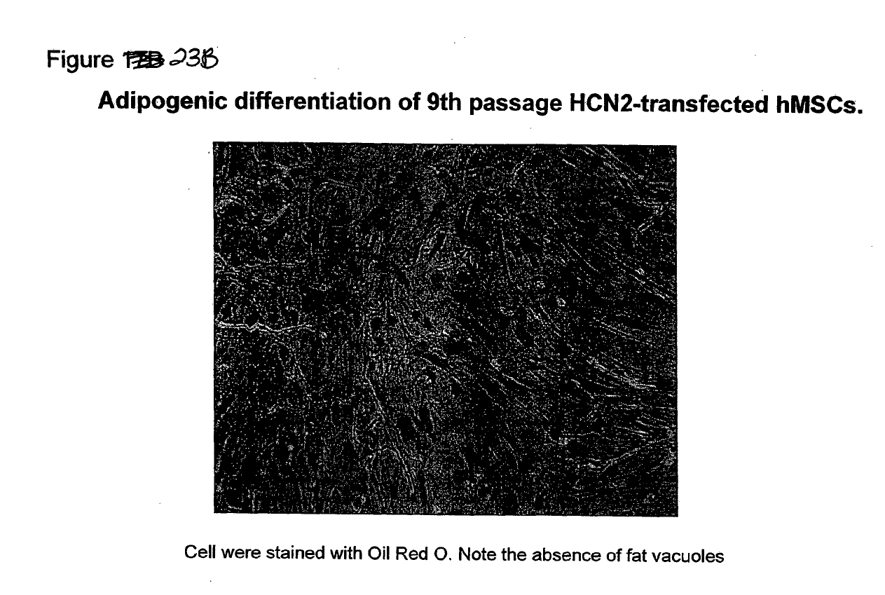

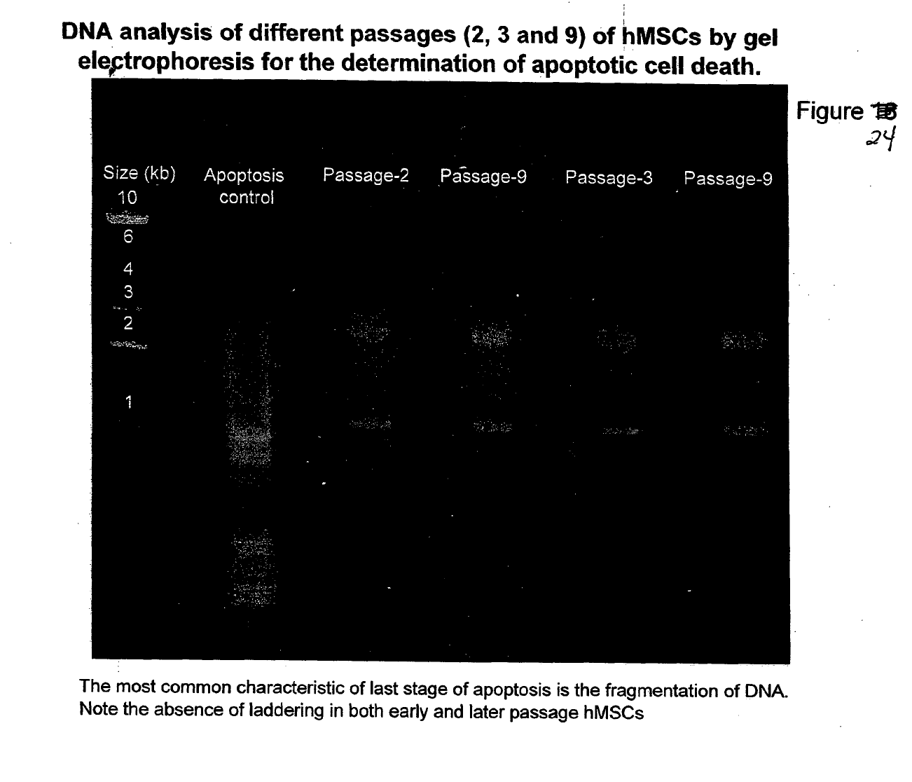

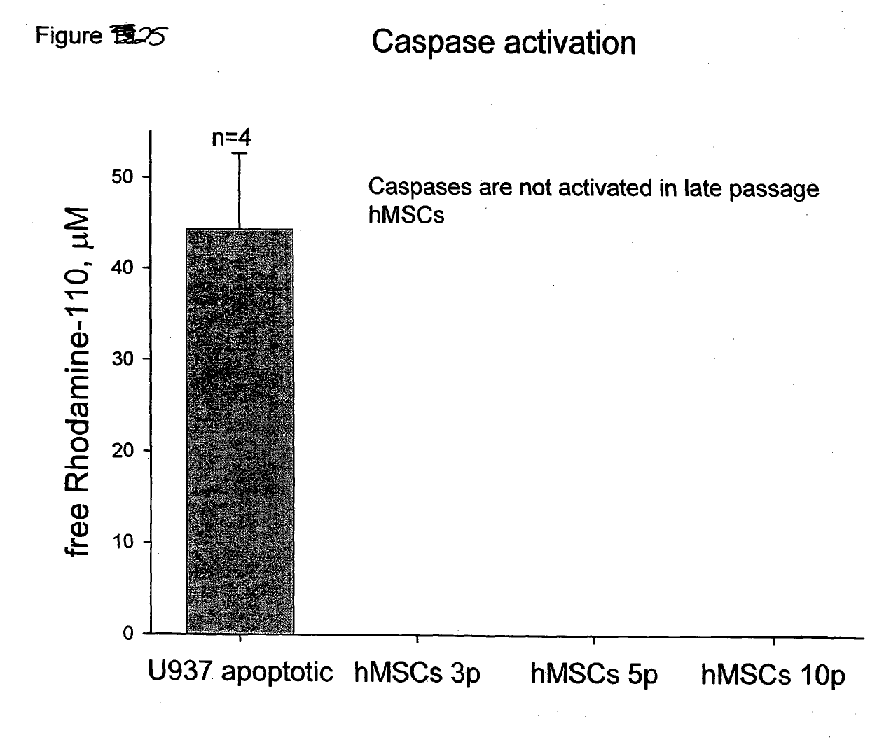

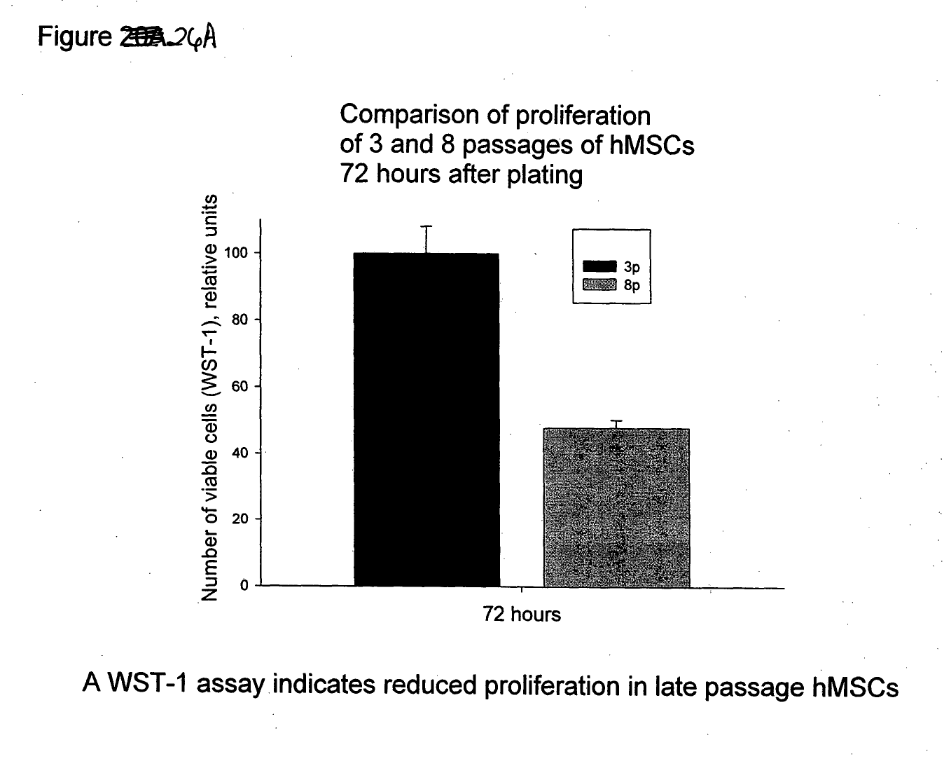

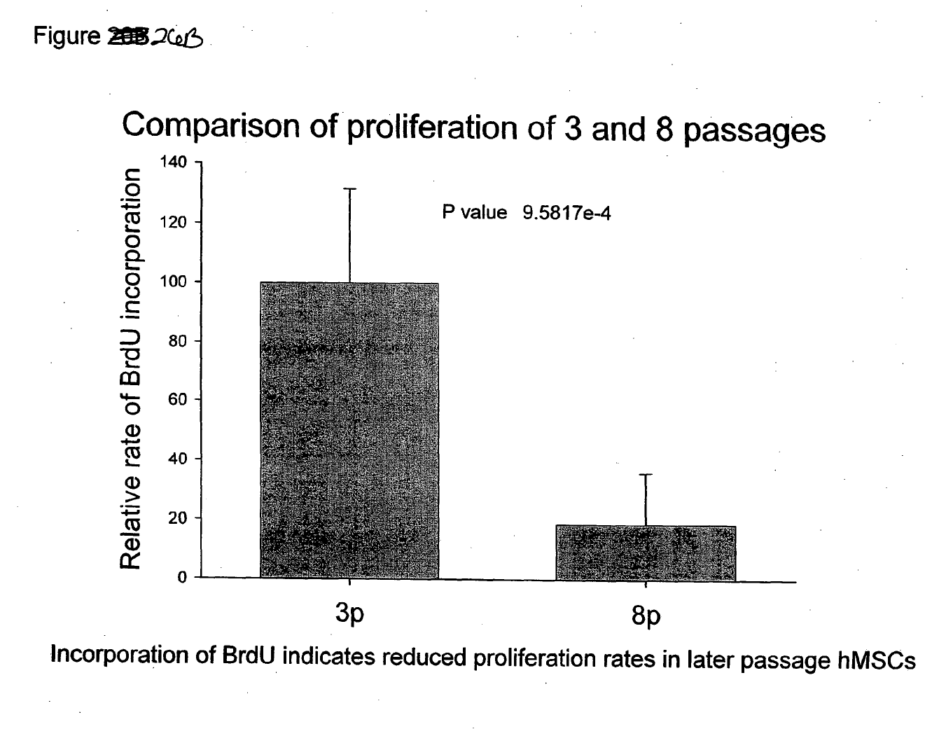

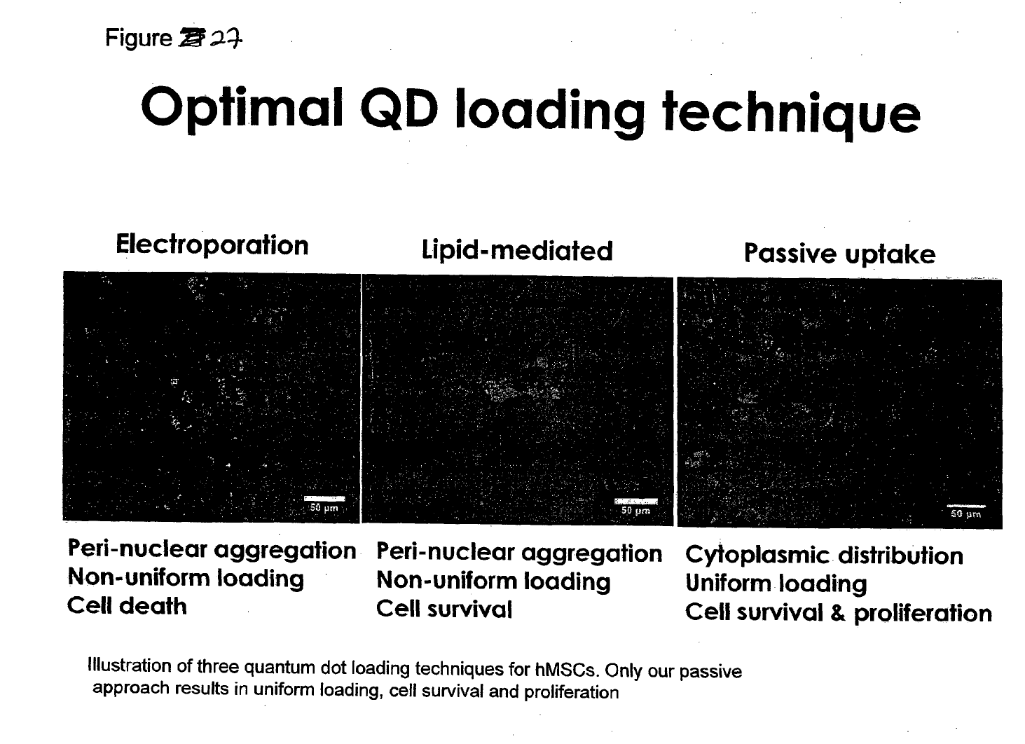

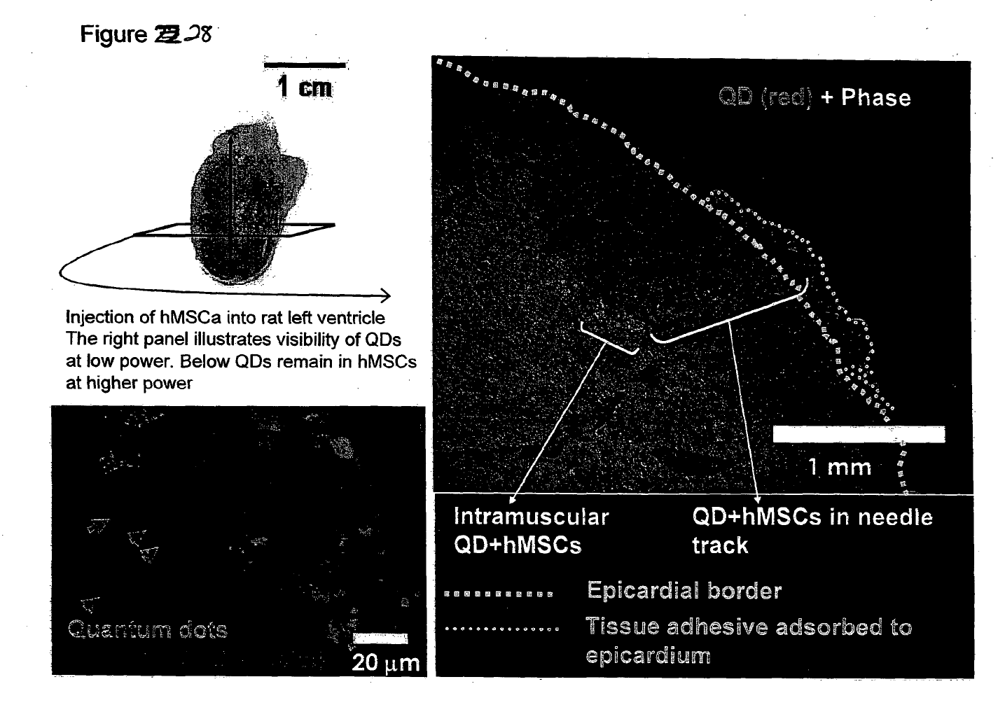

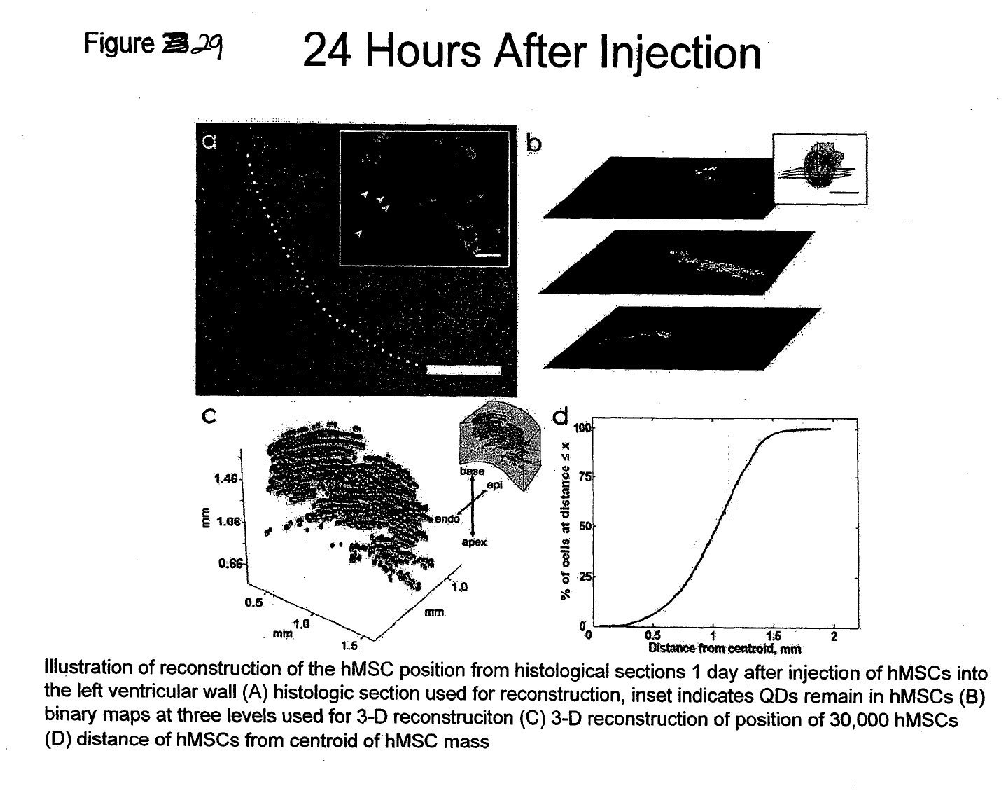

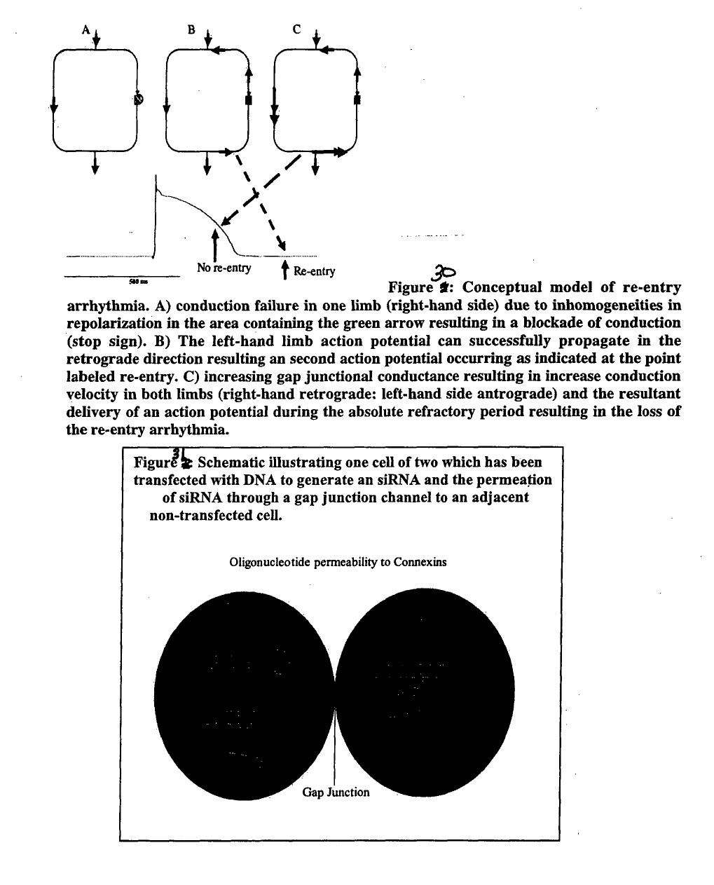

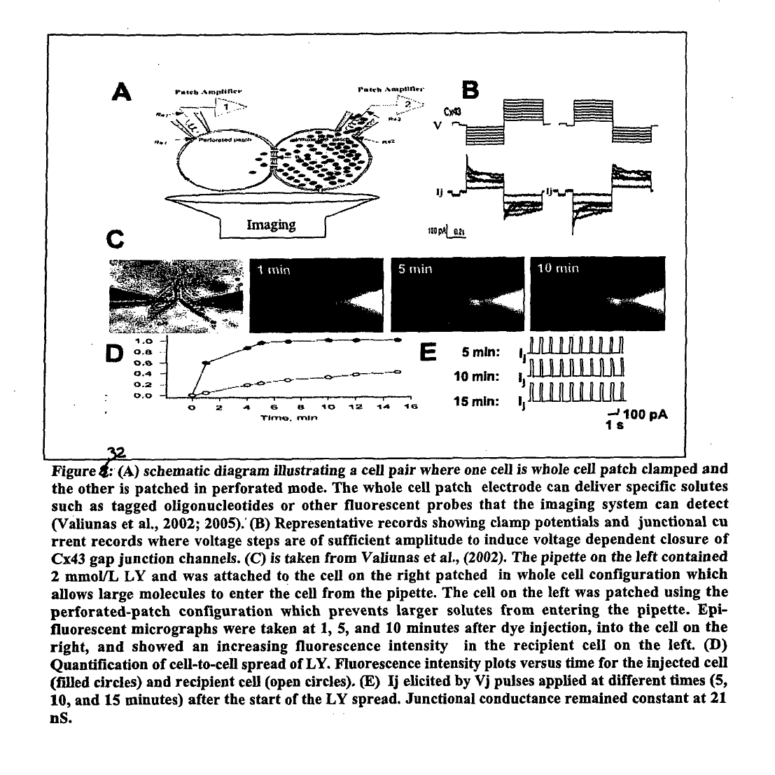

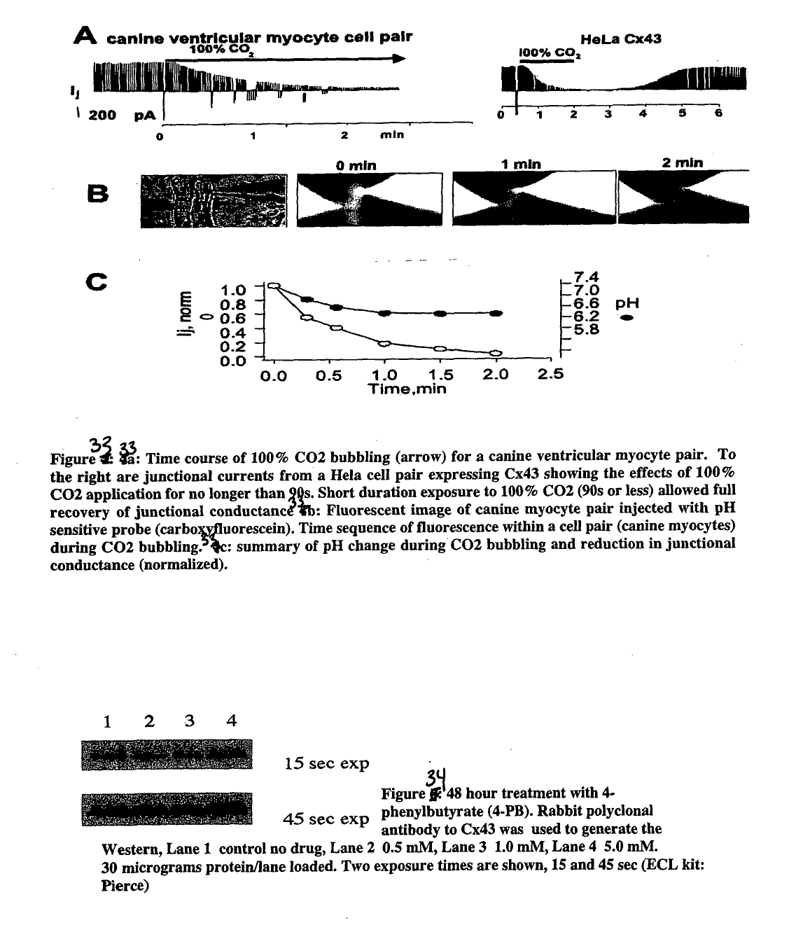

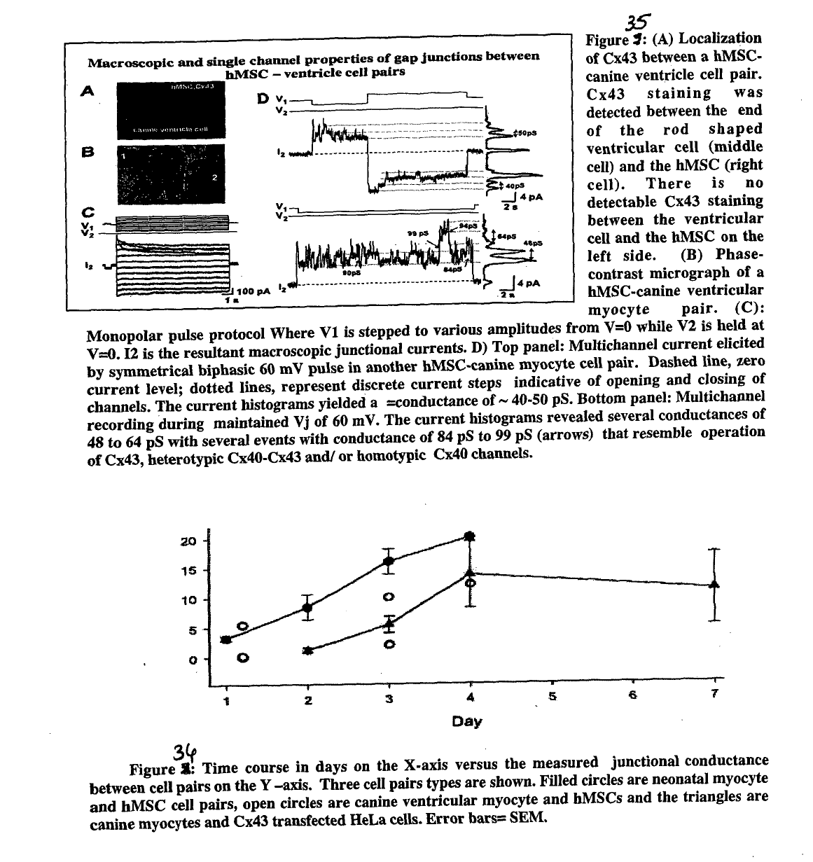

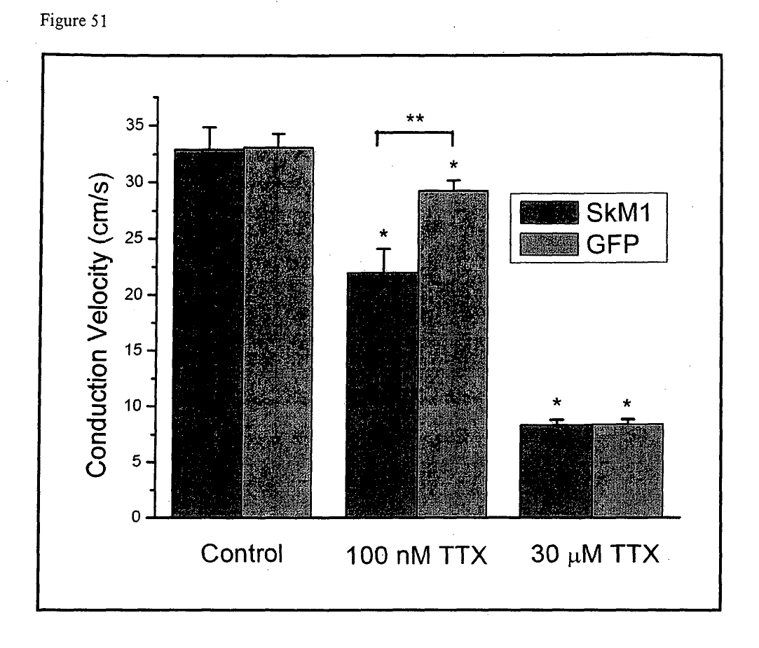

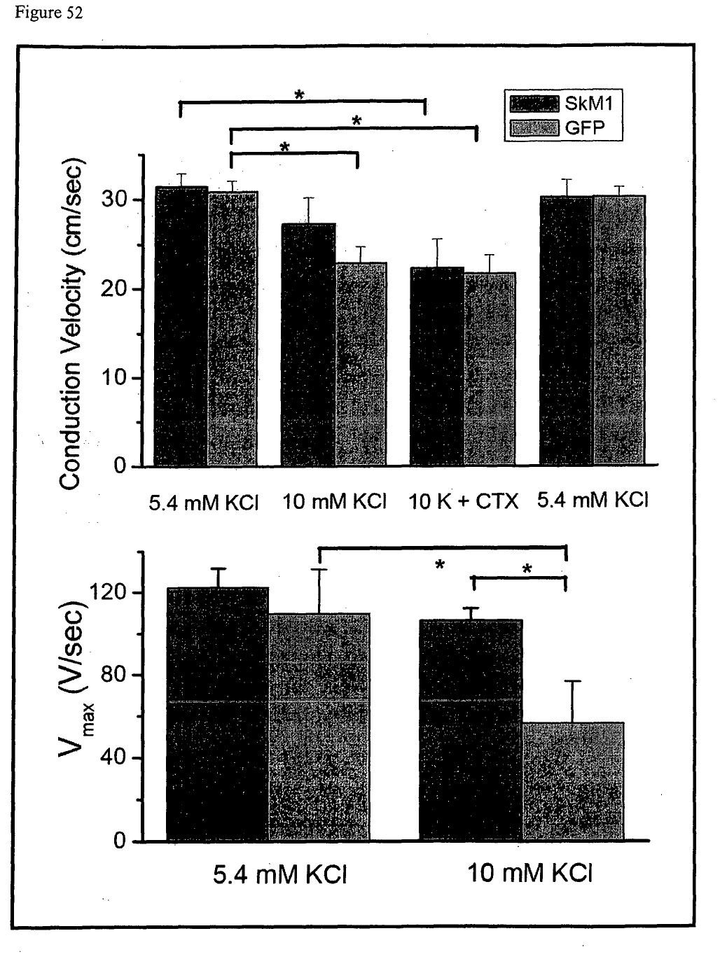

Next, a cell culture model of neonatal rat ventricle cells was used to examine the effect of low and high TTX concentrations on CV in cells expressing SkM1 in comparison to controls expressing GFP. The functional significance of the positive inactivation relation of the combined Na current is illustrated in FIG. 51, which indicates that expression of SkM1 impacts CV. Low (100 nM) and high (30 μM) concentrations of TTX were used to test the sensitivity of SkM1 expressing cultures versus GFP expressing cultures, with adenovirus used for delivery in each case. In GFP cultures there is a small but significant effect of 100 nM TTX on CV, which may reflect the presence of endogenous neuronal Na channel isoforms. When SkM1 is expressed, there is a significantly greater effect of low TTX on CV, reflecting the contribution of the exogenous TTX-sensitive SkM1 Na channel to CV. The CV is reduced to the same extent by high TTX in both SkM1 and GFP expressing cultures, illustrating that other contributors to CV are not significantly altered by SkM1 expression. The contribution of SkM1 to CV should indicate less sensitivity of CV and the action potential upstroke to depolarization. This is confirmed in FIG. 3, which shows summary data for the effect of K depolarization on CV (top) and AP upstroke (bottom) in GFP expressing (control) and SkM1 expressing myocytes. Membrane depolarization significantly reduces CV and V′max of control cells, but not the SkM1 expressing cells, where V′max remains in the normal range (>100 V/s).

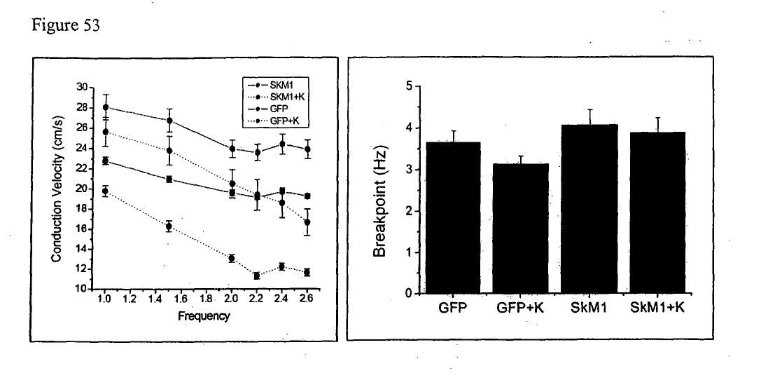

To determine if the gating characteristics of the expressed channel allow this protective effect to persist at higher frequencies, as may occur during reentrant arrhythmias, CV restitution relations in myocyte cultures expressing SkM1 Na channels+GFP and in control cultures (expressing GFP alone) was studied. FIG. 53 illustrates that CV remains greater as frequency increases, both in normal Tyrode's solution and in elevated K solution. Further, in the absence of SkM1, the breakpoint frequency is reduced in high K.

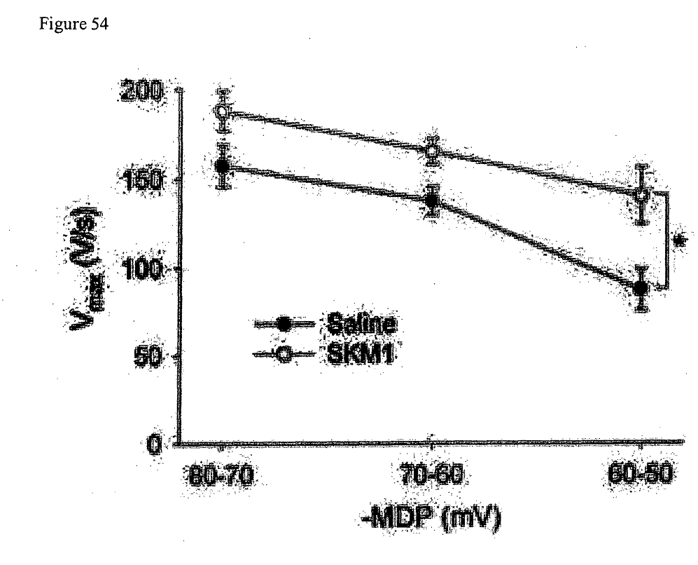

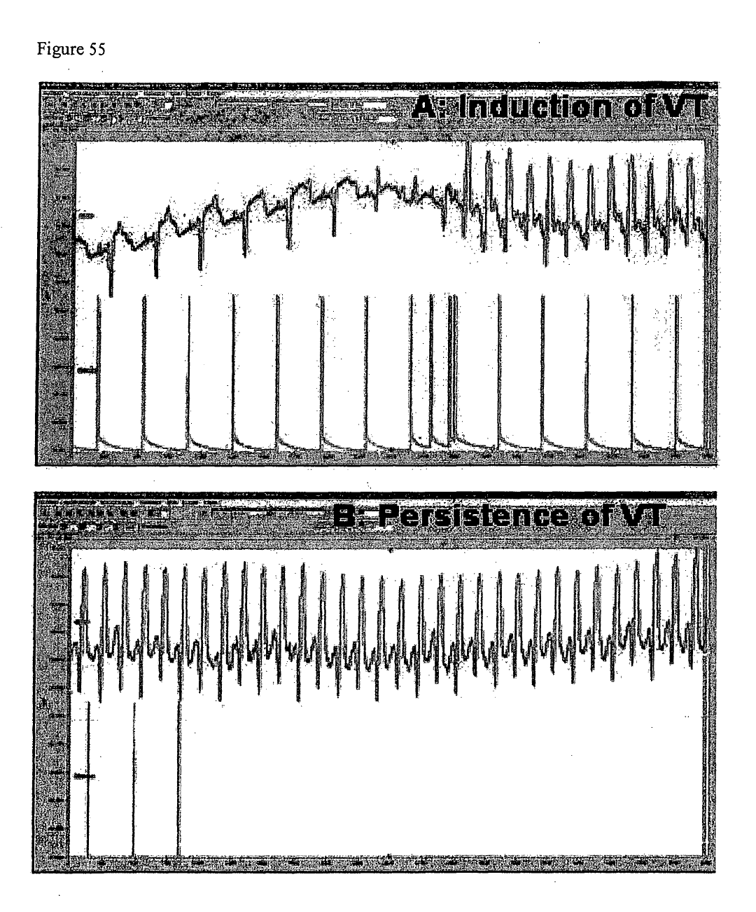

The effect of SkM1 overexpression via an adenoviral vector on antiarrhythmia in infarct-induced VT was examined. This experiment had several sections: first, the effect of SkM1 overexpression on the Na channel properties of depolarized myocytes was modeled and biophysically tested (see FIG. 50 and Table 4). Then, using a murine myocardial infarct model, the effect of intramyocardial SkM2 adenovirus injection on Vmax in depolarized myocytes was studied. FIG. 54 shows results 48 h post-infarct from 3 murine infarcts treated with adenovirus alone, and 3 with the SkM1 adenovirus. Results are “binned” into 10 mV sets. Throughout the range of membrane potentials, Vmax was higher with SkM1-treatment (P<0.05).

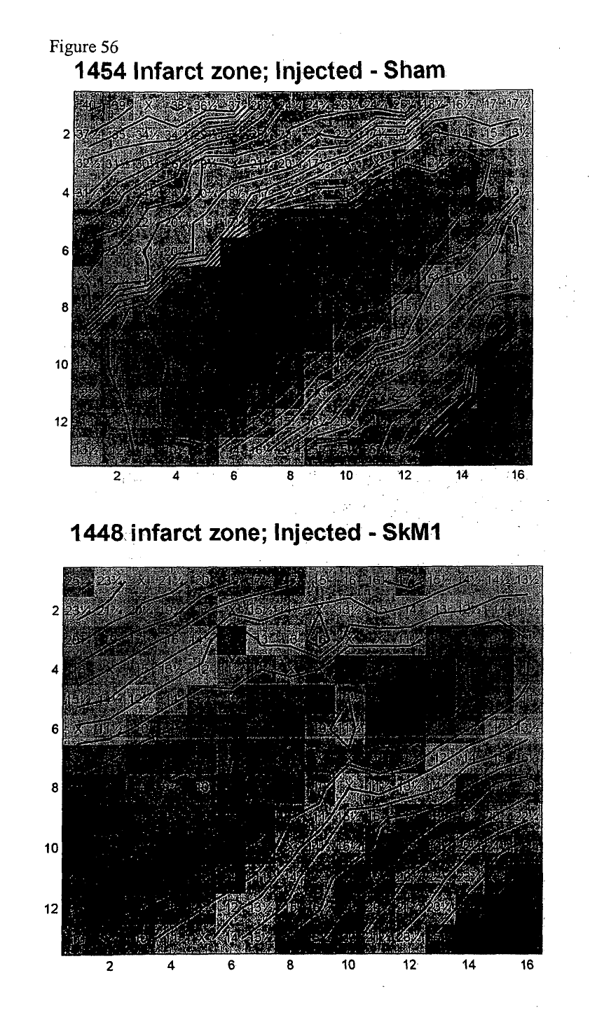

In Canine experiments, a GFP virus or a SkM1/GFP virus was administered subepicardially into a portion of the epicardial border zone region of 8 dogs with LAD occlusion. FIGS. 55, A and B, shows the ECGs from one dog injected with the GFP adenovirus. As shown here, during clinical EP testing monomorphic or polymorphic VT was routinely induced in infarcted animals. This occurred in 4 of 4 GFP-treated dogs and 0 of 3 SkM1-administered dogs (P<0.05). FIG. 55C uses epicardial mapping and shows that conduction can be enhanced (and normalized) in depolarized cells in a reentrant pathway by altering Na channel availability such that a greater number of Na channels can be activated from depolarized membrane potentials. To be emphasized here is that SkM1 appears to speed activation in the heart, in situ, when injected into the epicardial border zone. In addition, FIG. 56, shows isochronal maps from a dog receiving a GFP adenovirus (#1454) and another receiving the SkM1/GFP adenovirus (#1448), which demonstrate activation proceeding faster longitudinally than transversely in both settings, but also shows tighter isochronal spacing in the GFP-treated animal, consistent with slower activation.

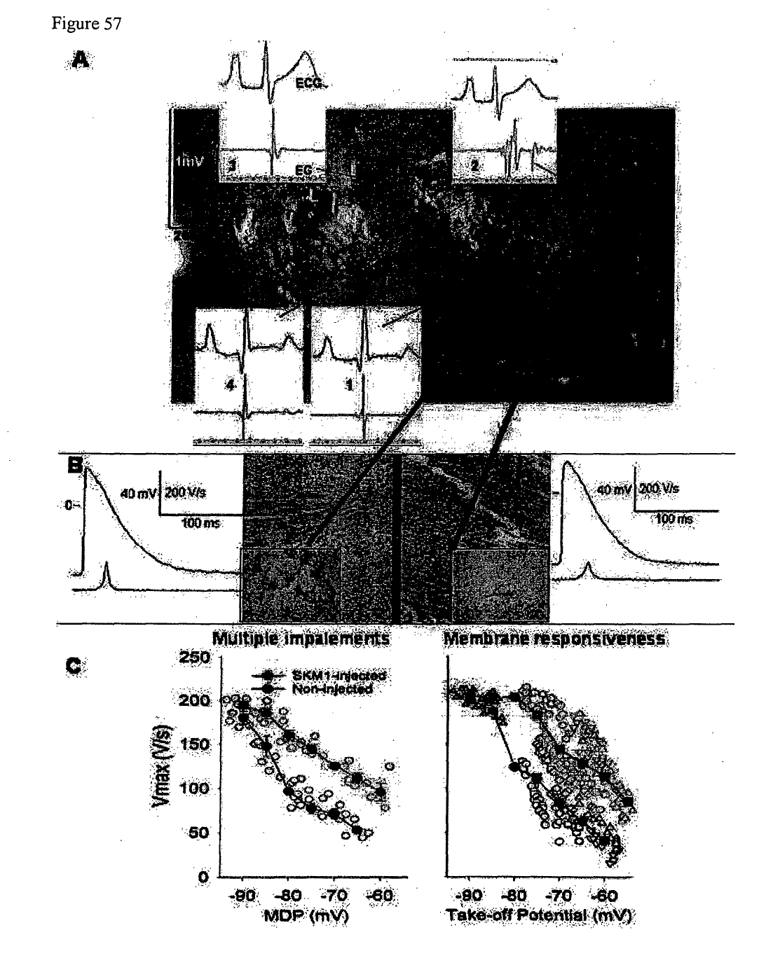

FIG. 57 presents electrogram and cellular electrophysiologic data from an SkM1-injected animal. Region 1 in Panel A is the epicardial border zone site of SkM1 injection. The electrogram here is a narrow spike, similar to those in the non-infarcted regions (exemplified by electrograms 3 and 4). In contrast at the non-injected epicardial border zone, site 2, the electrogram is broad and markedly fragmented, persisting into diastole. The heart was excised and 1 mm thick sections of sites 1 and 2 were removed for study in the tissue bath. Representative APs are shown in panel B. In both the sites there is equivalent membrane depolarization, but the tissue from site 1 (which received the SkM1) shows a Vmax nearly twice that of site 2. That sites 1 and 2 both represent infarcted regions is also shown in Panel B; moreover, the Panel B insets show that site 1 (which received SkM1) is GFP positive and site 2 is GFP negative. Panel C, left, demonstrates that a full range of membrane potentials was demonstrable at either site, but the Vmax was increasingly greater as membrane potential decreased in the SkM1 population. Panel C, right, shows membrane responsiveness curves for a subset of the same cells. Note that at the more positive potentials in the curves, there was a greater Vmax for the SkM1 injected site.

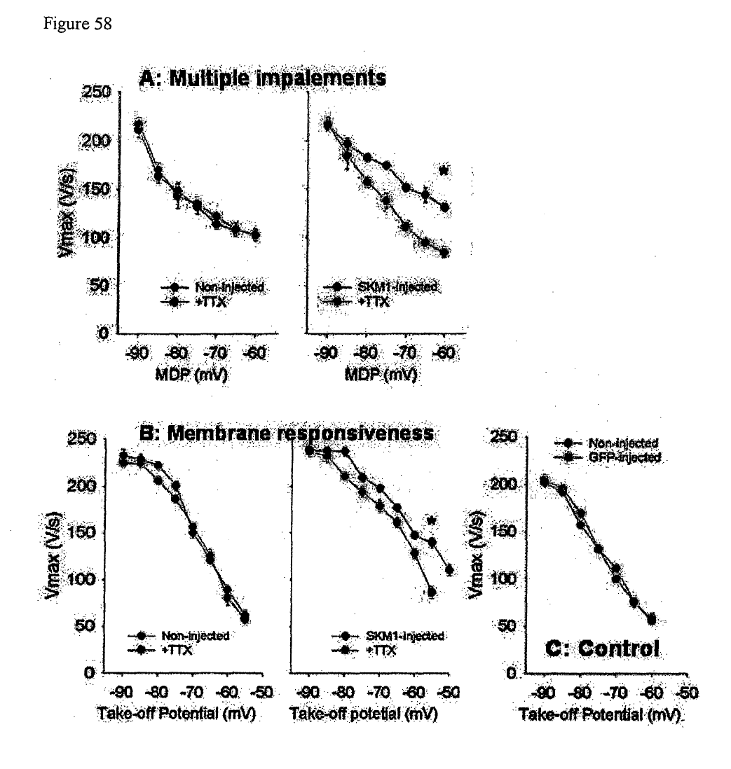

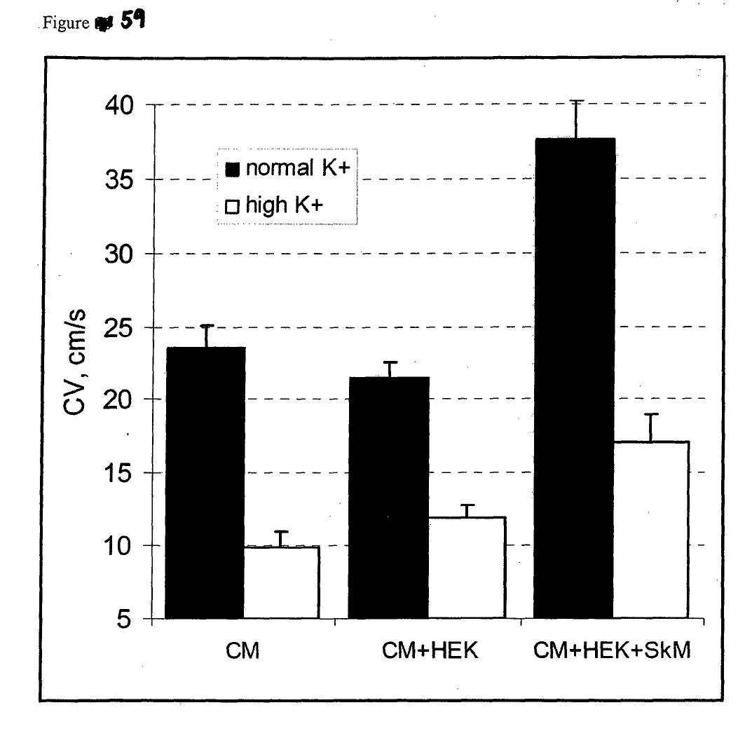

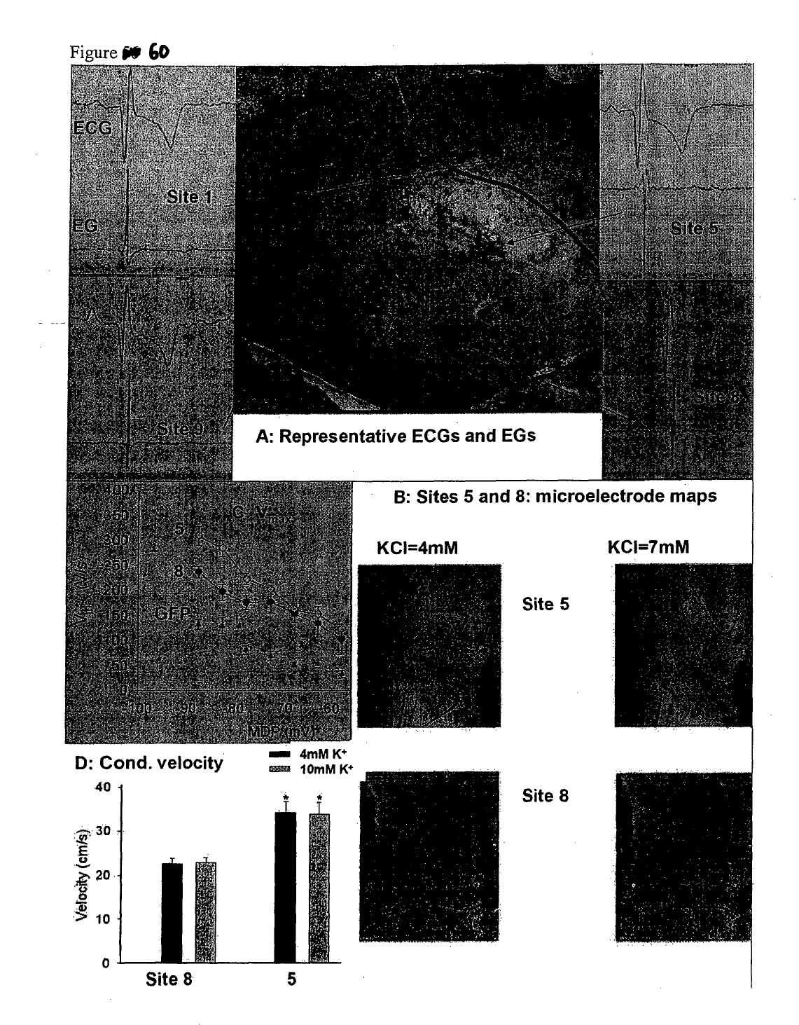

To test this whether the higher Vmax and membrane responsiveness in individual cell impalements and the more organized regional activation (seen as narrow, spiked electrograms) in the SkM1 treated regions truly derive from the SkM1 channel, the differential sensitivity of SkM1 versus (cardiac) SCN5A channels to TTX was utilized. Specifically, the former current should be significantly reduced by 100 nM TTX, while that of SCN5A channels should be minimally affected. This is tested in FIG. 58. In multiple impalements over a range of membrane potentials (Panel A) and in membrane responsiveness curves (Panel B) the SIM treated sites show a higher Vmax (especially at depolarized potentials) and a significant response to TTX, that is not seen in non-SkM1-treated tissues. In other words, the electrophysiologic function of the injected site is consistent with an SkM1 channel and that of the non-injected site is consistent with SCN5A. FIG. 58C shows that the adenovirus and GFP that accompany SkM1 do not affect membrane responsiveness. Also, there was no effect of virus+GFP on Vmax in multiple cell impalements (data not shown). Hence, it is the SkM1 construct rather than the associated virus or GFP that induces the effects on activation and Vmax reported here.

In sum, these experiments represent an effort in which (1) the effect of SkM1 on Na current was modeled and validated, (2) it was demonstrated that SkM1 has potentially beneficial actions in a relatively simple murine infarct model that would predict activity and antiarrhythmic efficacy in a model more applicable to man, and (3) the electrophysiological histological and histochemical imprints of the interventions used was shown. All these outcomes complement and extend the meaning of the in situ results and the results in cells; namely, that SkM1 can be delivered locally, has beneficial effects on activation, Vmax and membrane responsiveness and likely is antiarrhythmic. The above data relate to gene therapy approaches using Na channel constructs in viruses. Cell therapy approaches were also employed, where the Na channel was expressed in an adult mesenchymal stem cell (hMSC) that was then electrically coupled to the myocytes via gap junctions. To validate this approach in vitro, HEK293 cells that endogenously express Cx43 were used to create a cell line stably expressing SkM1 Na channels. For purposes of biophysical characterization and ease of study in culture, this cell line, with 100% of cells expressing the channel, is more convenient to work with than hMSCs. Co-cultures of these cells with neonatal myocytes were produced to measure CV in normal and high K (FIG. 10). When co-cultured with myocytes, HEK cells expressing SkM1 result in a greater CV than is seen with either control myocytes or co-cultures of myocytes with non-expressing HEK cells, and the effect of the SkM1 expression persists in high K. This result confirms that exogenous Na current can be delivered to myocytes via coupled non-myocyte cells and impact CV. Further, the co-culture of HEK cells not expressing SkM1 did not measurably reduce CV at the plating density employed (myocyte:HEK ratio of 9:1 at time of experiment).

In work on MSCs as a platform to carry SkM1 to the 5-day infarct, the experiments in FIG. 60 ware performed, which demonstrate that the stem cells can, in fact be used to carry constructs of interest effectively to the infarcted heart. The heavy line in FIG. 60 marks the upper margin of the infarct. Panel A shows ECGs and EGs from 4 representative sites. Site 5 received 700,000 hMSCs loaded with quantum dots and SkM1. Site 8 is a representative infarcted region that received no MSCs. Site 1 is outside the infarct and Site 9 is outside but at the edge. Note that both Sites 5 and 8 have EGs that are narrow. Panel B shows microelectrode maps of sites 5 and 8 demonstrating that at K+=4 mM the isochrones (5 ms) are comparable and that they do not become more tightly packed at 7 mM. Panel C shows the relationship of Vmax to membrane potential for 4 infarcted animals that received a GFP virus in comparison to Sites 5 and 8 (39-41 impalements/site). Site 5 has the highest curve and both Sites 5 and 8 differ from the GFP curve. Panel D: conduction velocity at K=4 and 10 mM: At Site 5 at both [K+] propagation is faster than at Site 8; velocity does not decrease as [K+] increases.

1. A method of treating atrial fibrillation comprising modifying the conducting properties of the tissues in which the impulses are propagating, wherein the method improves conduction in the tissues by increasing gap junctional conductance in said tissues.

2. The method of claim 1, wherein said tissues comprise the atrium.

3. The method of claim 1, wherein the method comprises delivering to said tissues hMSCs transfected with endogenous heart connexins.

4. The method of claim 3, wherein said connexins are Cx40, Cx43 or Cx45.

5. The method of claim 1, wherein the method comprises delivering to said tissues a viral vector capable of expressing endogenous heart connexins in said tissue.

6. The method of claim 5, wherein said viral vector is derived from a lentivirus and wherein said connexin is selected from the group consisting of Cx40, Cx43 and Cx45.

7. The method of claim 1, wherein the method comprises administering a chemical stimulator of connexin expression to said tissues to cause said tissues to overexpress endogenous connexins selected from the group consisting of Cx 40, 43, 45.

8. The method of claim 7, wherein said chemical stimulator is 4PB or Zp123.

9. The method of claim 1, wherein the method comprises administering a MMP-7 inhibitor to said tissues to inhibit MMP-7 in said tissue to cause said tissues to overexpress endogenous connexins selected from the group consisting of Cx 40, 43, and 45.

10. The method of claim 9, wherein the MMP-7 inhibitor is Gefitinib.

11. The method of claim 1, wherein the method comprises delivering to said tissues hMSCs transfected with exogenous connexins selected from the group consisting of Cx46 and Cx32.

12. The method of claim 11, wherein the method comprises administering to said tissues a viral vector capable of expressing an exogenous heart connexin in said tissue, wherein the exogenous connexin is selected from the group consisting of Cx46 and Cx32.

13. The method of claim 12, wherein said viral vector is derived from a lentivirus.

14-52. (canceled)

53. A method of treating atrial fibrillation comprising modifying the conducting properties of the cells in which reentry is taking place, wherein the method reduces conduction in the cells by downregulating the alpha subunit of SCN5a.

54. The method of claim 53, wherein downregulating SCN5a comprises administering SCN5a alpha subunit siRNA to all or part of the atrium.

55. The method of claim 54, wherein the SCN5a alpha subunit siRNA is delivered using a viral vector.

56. The method of claim 54, wherein the SCN5a alpha subunit siRNA is delivered using a cellular carrier.

57. The method of claim 56, wherein the cellular carrier is a human mesenchymal stem cell.

58. A composition comprising a cell that expresses SCN5A siRNA.

59. The composition of claim 58, wherein the cell is a human mesenchymal stem cell.

60. (canceled)
